(19) United States
(12) Patent Application Publication
(10) Pub. No.: US 2026/0002163 A1
(43) Pub. Date: Jan. 1, 2026

(54) COMPOSITIONS AND METHODS FOR TREATING PANCREATIC CANCER

(71) Applicant: Molecular Axiom, LLC, San Diego, CA (US)

(72) Inventors: Lishan CHEN, San Diego, CA (US); Bohan JIN, San Diego, CA (US); Yuching CHEN, San Diego, CA (US); Rosaline TRUONG, San Diego, CA (US)

(21) Appl. No.: 18/880,903

(22) PCT Filed: Jul. 6, 2023
(86) PCT No.: PCT/US2023/026980

§ 371 Date: Jan. 3, 2025

Related U.S. Application Data
(60) Provisional application No. 63/358,588, filed on Jul. 6, 2022.

(51) Int. Cl. C12N 15/115; A61P 35/00; C12N 15/113; C12N 15/88

(52) U.S. Cl. CPC C12N 15/115; A61P 35/00; C12N 15/113; C12N 15/1137; C12N 15/88; C12Y 306/05002; C12N 2310/11; C12N 2310/16; C12N 2310/315; C12N 2310/321; C12N 2310/322; C12N 2310/3231; C12N 2310/341

(57) ABSTRACT

In the various aspects and embodiments, the present disclosure provides compositions and methods for treating pancreatic cancer (e.g., pancreatic ductal adenocarcinoma, or PDAC). In accordance with aspects of the disclosure, the composition comprises an aptamer that targets accumulation of the composition to pancreatic cancer cells, and an antisense oligonucleotide that inhibits the expression of an mRNA associated with key signaling pathways that promote proliferation or survival in pancreatic cancer cells, such as the KRAS-RAF-MEK-ERK signaling pathway or RTK-RAS-ERK cascade. Exemplary antisense oligonucleotides described herein target KRAS, including mutant KRAS. Exemplary antisense oligonucleotides described herein target SOS1 and/or SOS2 transcripts.

### PRIORITY

This application claims the benefit of, and claims priority to, U.S. provisional application No. 63/358,588, filed Jul. 6, 2022, which is hereby incorporated by reference in its entirety.

### BACKGROUND

Pancreatic ductal adenocarcinoma (PDAC) is a lethal malignancy with limited treatment options. Activating mutations of the KRAS GTPase are the predominant dependency present in >90% of PDAC patients. The need for new therapies targeting the RAS genes, particularly KRAS, and the proteins that regulate Ras activity, is of high priority. In the various aspects and embodiments of this disclosure compositions and methods for treating pancreatic cancer are provided.

### BRIEF DESCRIPTION OF THE DRAWINGS

FIG. 1 illustrates knockdown of KRAS mRNA encoding a mutated KRAS protein comprising the G12C mutation, mediated by antisense oligonucleotides.

FIG. 2 illustrates knockdown of expression of wild type or mutated KRAS protein (G12C mutation) mediated by an antisense oligonucleotide (ASO 28) (see Table 3).

FIG. 3 illustrates knockdown of expression of mutated KRAS protein (G12C mutation) mediated by antisense oligonucleotides (ASO 28 or ASO 67) (see Table 3).

FIG. 4 illustrates three-dimensional (3D) cell proliferation inhibition due to inhibiting expression of mutated KRAS by contacting the cell harboring the KRAS mutation (NCI-H358 cell having a KRAS G12C mutation) with an antisense oligonucleotide.

FIG. 5 illustrates knockdown of KRAS mRNA encoding a mutated KRAS protein comprising the G12V mutation mediated by an antisense oligonucleotide.

FIG. 6 illustrates knockdown of expression of mutated KRAS protein (LCLC97TM1 cell having the G12V mutation) mediated by an antisense oligonucleotide (ASO 80, see Table 3).

FIG. 7 illustrates knockdown of expression of mutated KRAS protein (LCLC97TM1 cell having the G12V mutation) mediated by an antisense oligonucleotide (ASO 81, see Table 3).

FIG. 8 illustrates 3D cell proliferation inhibition by inhibiting expression of mutated KRAS by contacting the cell harboring the KRAS mutation (LCLC97TM1 cell, NCI-H441 cell, or CFPAC-1 cell having the G12V mutation) with an antisense oligonucleotide.

FIG. 9 illustrates 3D cell proliferation inhibition by inhibiting expression of mutated KRAS by contacting the cell harboring the KRAS mutation (NCI-H2009 cell or SW1116 cell having the G12A mutation) with an antisense oligonucleotide.

FIG. 10 illustrates knockdown of KRAS mRNA encoding a mutated KRAS protein comprising the G12D mutation mediated by an antisense oligonucleotide.

FIG. 11A-11C illustrates P19 aptamer specificity for PDAC cells compared to non-PDAC cells by fluorescent microscopy.

FIG. 12 illustrates knockdown of KRAS mRNA encoding a mutated KRAS protein comprising the G12C mutation mediated by P19 aptamer-antisense oligonucleotides with different linker structures.

FIG. 13 compares knockdown of KRAS mRNA in MIA PaCa-2 cells mediated by an antisense oligonucleotide with and without linkage to P19 aptamer. P19-conjugated ASO employs a 2x C3 spacer.

FIG. 14A-14B illustrates 3D cell proliferation inhibition by inhibiting expression of mutated of KRAS mRNA encoding a mutated KRAS protein comprising the G12C mutation in MIA PaCa-2 cells mediated by an antisense oligonucleotide with and without linkage to P19 aptamer. For FIG. 15A compounds were contacted with cells for 6 days. For 15B, compounds were contacted with cells for one hour, then washed out and replenished with medium for 6 days.

FIG. 15 illustrates knockdown of SOS1 mRNA in MIA PaCa-2 cells mediated by an antisense oligonucleotide with or without linkage to P19 aptamer.

FIG. 16 illustrates knockdown of SOS1 mRNA in MIA PaCa-2 cells mediated by an antisense oligonucleotide with or without linkage to P19 aptamer.

FIG. 17 illustrates 3D cell proliferation inhibition by inhibiting expression of mutated of SOS1 mRNA in MIA PaCa-2 cells (PDAC) mediated by antisense oligonucleotide with or without linkage to P19.

FIG. 18 illustrates 3D cell proliferation inhibition by inhibiting expression of SOS1 mRNA in MIA PaCa-2 and NCI-H1975 (NSCLC) cells mediated by antisense oligonucleotide with or without linkage to P19.

### DETAILED DESCRIPTION

In the various aspects and embodiments, the present disclosure provides compositions and methods for treating pancreatic cancer (e.g., pancreatic ductal adenocarcinoma, or PDAC). In accordance with aspects of the disclosure, the composition comprises an aptamer that targets accumulation of the composition to pancreatic cancer cells, and an antisense oligonucleotide that inhibits the expression of an mRNA associated with key signaling pathways that promote proliferation or survival in pancreatic cancer cells, such as the KRAS-RAF-MEK-ERK signaling pathway or RTK-RAS-ERK cascade.

PDAC is one of the most lethal human cancers, with a 5-year survival rate of less than about 7%. A large proportion of patients die within 6 months after diagnosis. PDAC frequently harbors a KRAS mutation. KRAS (Kirsten rat sarcoma virus) or K-Ras is part of the RAS/MAPK pathway. The K-Ras protein is a GTPase that is activated by the binding of GTP. The K-Ras protein is inactivated when it converts the GTP to GDP. When the protein is bound to GDP, it does not relay signals to the cell's nucleus. In normal quiescent cells, K-Ras is predominantly GDP-bound and inactive. Upon activation of receptor tyrosine kinases (RTKs) there is a transient formation of K-Ras-GTP, which regulates numerous intracellular signaling networks and controls mitogenic processes. K-Ras can also bind to proteins of the Guanine Nucleotide Exchange Factor (GEF) class (such as SOS1), which forces the release of bound nucleotide (GDP) from K-Ras. A single amino acid substitution in K-Ras is responsible for an activating mutation in various malignancies, including lung adenocarcinoma, mucinous adenoma, ductal carcinoma of the pancreas, and colorectal cancer. Frequent driver mutations include G12 substitutions, which render K-Ras persistently GTP-bound and constitutively active. K-Ras is a main component of the KRAS-RAF-MEK-ERK signaling pathway, which is a critical regulator of cell proliferation. See Waters AM and Der CJ, KRAS: The Critical Driver and Therapeutics Target for Pancreatic Cancer, Cold Spring Harb. Perspect. Med. 2018.

Further, the RTK-Ras-ERK cascade is a central signaling module implicated in the control of biological processes including cell proliferation and survival. The coupling of RTK to Ras is mediated by the Ras-specific nucleotide-exchange factor Son of Sevenless (Sos), which activates Ras by inducing the exchange of GDP for GTP. A positive feedback loop involving Ras-GTP and Sos leads to an increase in the amplitude and duration of Ras activation in response to EGF stimulation.

In various aspects and embodiments, the compositions of the present disclosure comprise RNA aptamers associated with antisense oligonucleotides that inhibit expression of mRNA associated with key signaling pathways, such as the KRAS-RAF-MEK-ERK signaling pathway or RTK-RAS-ERK pathway in pancreatic cancer cells. Without wishing to be bound by theory, the aptamer may target a cell surface molecule or an endocytic membrane associated protein (e.g., a membrane receptor or a glycoprotein) that is overexpressed on pancreatic cancer cells or is specifically expressed on pancreatic cancer cells. The associated antisense oligonucleotide inhibits expression of an endogenous nucleic acid (e.g., an mRNA) in a pancreatic cancer cell that is required for the KRAS-RAF-MEK-ERK signaling pathway or RTK-RAS-ERK signaling pathway. The present disclosure contemplates compositions involving conjugations between the aptamer and antisense oligonucleotide, or encapsulation of the antisense oligonucleotide in aptamer-decorated particles. As used herein, the term “conjugated to,” or “conjugate” refers to two or more entities or the state of two or more entities being linked by a direct or indirect covalent or non-covalent interaction. In some embodiments, a conjugation is via covalent interaction.

In accordance with this disclosure, the aptamer comprises a nucleotide sequence that targets accumulation of the composition to pancreatic cancer cells, thereby overcoming limitations of prior approaches to target mutated K-Ras and/or associated signaling cascades, which lacked sufficient efficacy or potency or demonstrated off-target effects. In some embodiments, the aptamer comprises the nucleotide sequence GAAUGCCC (SEQ ID NO: 1003). Exemplary aptamers comprise the nucleotide sequence CUCAAUGGCGAAUGCCCGCCUAAUAGGG (SEQ ID NO: 1004) or a derivative thereof. In some embodiments, the aptamer comprises the nucleotide sequence: GGGAGACAAGAAUAAACGCUCAAUGGCGAAUGCCCGCCUAAUAGGGCGUUA UGACUUGUUGAGUUCGACAGGAGGCUCACAACAGGC (SEQ ID NO: 1005) or a derivative thereof. In some embodiments, the aptamer comprises from 1 to about 20, or from 1 to about 15, or from 1 to about 10, or from 1 to 5 nucleobase substitutions with respect to SEQ ID NO: 1004 or 1005. In some embodiments, the substitutions are modified nucleobases (for example, modified U for U, modified A for A, modified G for G, or modified C for C) as known in the art. Aptamers and derivatives are described in U.S. Pat. Nos. 10,550,394, 11,261,449 and 9,464,293, each of which are hereby incorporated by reference in their entireties. In various embodiments, the aptamer has a nucleotide sequence (as described above) that is about 100 nucleotides in length or less, or about 88 nucleotides in length or less, or about 80 nucleotides in length or less, or about 70 nucleotides in length or less, or about 60 nucleotides in length or less, or about 50 nucleotides in length or less, or about 40 nucleotides in length or less, or about 30 nucleotides in length or less. In various embodiments, the aptamer is at least about 20 nucleotides in length or at least about 25 nucleotides in length, or at least about 28 nucleotides in length. For example, in various embodiments, the aptamer has a length of from about 25 nucleotides to about 80 nucleotides, such as from 25 nucleotides to about 60 nucleotides, or from about 25 nucleotides to about 40 nucleotides in length.

In various embodiments, the aptamer nucleotide sequence can be chemically modified. In some embodiments, the aptamer comprises RNA nucleotides, that is, nucleotides having a 2′ hydroxyl. In some embodiments, the aptamer may comprise non-RNA nucleotides modified at the 2′ position. For example, the aptamer nucleotide sequence may comprise one or more (or all or substantially all) pyrimidines modified with 2′-Fluoro, (i.e., fU or fC) which in some embodiments can enhance nuclease resistance and/or aptamer folding. In some embodiments, the aptamer comprises one or more additional chemical modifications described herein, including 2′ modifications (e.g., 2′-O methyl, 2′-O ethyl, 2′-O methoxyethyl (MOE), and a bridged nucleotide having a 2′ to 4′ bridge such as LNA or cEt), backbone modifications (e.g., phosphorothioate or phosphorodithioate), or nucleobase modifications (i.e., modified nucleotides) as are known in the art. See U.S. Pat. No. 10,064,959, which is hereby incorporated by reference. In some embodiments, the aptamer is constructed without backbone modification.

In some embodiments, the aptamer has the structure 5′ [fC][fU][fC]AA[fU][GG][fC]GAA[fU]G[fC][fC]G[fC][fC][fU]AA[fU]AGGG 3′ (SEQ ID NO: 1006) and is designated herein as P19. In this structure, “f” designates a 2′ fluoro nucleotide.

In some embodiments, the aptamer comprises one or more hydrocarbon linkers (e.g., an alkylene) or a polyether linker (e.g., a PEG linker) inserted between one or more nucleotides. In some embodiments, nucleotides of the aptamer may be replaced with a hydrocarbon linker or a polyether linker (e.g., replacing from 2 to 10 nucleotides) provided that the binding or selectivity of the aptamer for pancreatic cancer cells is not substantially reduced (e.g., by more than 20% or by more than 10%) by the substitution. In some embodiments, nucleotides are replaced with the linker that are not present in a core motif. Exemplary spacer moieties are described herein, and may be oligoethylene glycol moieties providing 3 to 18 atom spacers. Aptamers with spacers can be synthesized using phosphoramidite spacer moieties as known in the art.

In various embodiments, the compositions of the present disclosure inhibit expression of the KRAS gene, which is one of the four main driver genes (KRAS, TP53, CDKN2A and SMAD4) in PDAC. As a member of the RAS gene family, the KRAS protein (21 kDa) has GTPase activity and thus binds GTP in the activated state and GDP in the deactivated state. Ras regulates cell proliferation, differentiation, and apoptosis by activating several signaling pathways, including the RAF/MEK/ERK, PI3K/AKT/mTOR, PLC/PKC, and RAL pathways.

In some embodiments, the antisense oligonucleotide targets KRAS mRNA. Exemplary nucleobase sequences for antisense oligonucleotides targeting KRAS are shown in Table 1. In some embodiments, the antisense oligonucleotide comprises or consists of a nucleotide sequence listed in Table 1 or Table 2. In some embodiments, the antisense oligonucleotide targets WT and mutant KRAS isotype mRNAs, that is, the oligonucleotide is not specific for an mRNA encoding a mutant form of KRAS. In these embodiments, the aptamer delivers the antisense oligonucleotide selectively to cancer cells, thereby avoiding any toxicity in normal cells due to loss of KRAS expression, and avoiding genetic testing of a patient's KRAS mutation.

In some embodiments, the antisense oligonucleotide targets a mutant KRAS mRNA (e.g., encoding a G12 mutant), and such antisense oligonucleotides may comprise or consist of a nucleotide sequence shown in Table 3. In still other embodiments, the oligonucleotide comprises or consists of a sequence listed in Table 4. For simplicity nucleotide sequences may be shown herein using DNA nucleotide sequences (i.e., including T nucleobases) or as RNA nucleotide sequences (i.e., including U nucleobases). It is understood from the context (unless otherwise stated) that when the nucleotide or sequence is intended to be RNA, T nucleotides are substituted as U (or modified U such as pseudouridine or 1-methylpseudouridine), and vice versa in the case of DNA.

In various embodiments, the antisense oligonucleotide comprises at least 8, or at least 10, or at least 12 contiguous nucleotides of a nucleotide sequence shown in Table 1, Table 2, Table 3, or Table 4. In some embodiments, the antisense oligonucleotide consists of a nucleotide sequence selected from Table 13. In some embodiments, the antisense oligonucleotide has from 13 to 24 linked nucleotides (e.g., 13 to 18, or 13 to 16, or 14 to 16 consecutive nucleotides) of (or comprising) a sequence disclosed in Table 1, Table 2, Table 3, or Table 4.

Exemplary antisense oligonucleotides comprise or consist of a nucleotide sequence selected from Table 3. Antisense oligonucleotides (ASO) constructed from sequences in Table 3 are constructed as gapmers. For example, unless otherwise indicated in Table 3, nucleotide sequences are constructed as 3-X-3 gapmers with three locked nucleotides (LNAs) on each termini. Antisense oligonucleotides are fully phosphorothioate linked. The ASO may alternatively be constructed according to other chemistries profiles described herein.

Exemplary antisense oligonucleotides comprise or consist of a nucleotide sequence selected from Table 4. Antisense oligonucleotides constructed from sequences in Table 4 may also be constructed as gapmers. For example, nucleotide sequences can be constructed as 3-X-3 gapmers with three locked nucleotides (LNAs) on each termini. Antisense oligonucleotides can be fully phosphorothioate linked. The ASO may alternatively be constructed according to other chemistries profiles described herein.

Exemplary nucleotide sequences and antisense oligonucleotides targeting KRAS (wild-type or mutated) are described in WO 2023/034537 and U.S. provisional application No. 63/480,467, which are hereby incorporated by reference in their entireties. Such nucleotide sequences and antisense oligonucleotides may be used in connection with the present disclosure.

In some embodiments, the antisense oligonucleotide targets KRAS mRNA encoding a mutant KRAS. In various embodiments, the mutant KRAS is selected from a G12C mutation, a G12V mutation, a G12A mutation, and a G12D mutation. For example, the antisense oligonucleotide may comprise or consist of a nucleotide sequence selected from Table 3 or Table 4.

In some embodiments, the antisense oligonucleotide selectively targets and inhibits SOS1 and/or SOS2 expression, thereby preventing the interaction of SOS1 with KRAS in the guanosine diphosphate (GDP)-bound ‘off’ state, which is the inactivated state of KRAS.

In some embodiments, the antisense oligonucleotide targets (e.g., hybridizes to) SOS1 and/or SOS2 mRNA. Son of Sevenless 1 and 2 (SOS1 and SOS2) promote RAS activation. In some embodiments, the oligonucleotide targets SOS1, transcript variant 2 (NM_001382394.1), transcript variant 3 (NM_001382395.1), or transcript variant 1 (NM_005633.4). In some embodiments, the antisense oligonucleotide targets SOS2 mRNA (NM_006939.4). In some embodiments, the antisense oligonucleotide is complementary to SOS1 or SOS2, but has significant activity against the other mRNA as well. Nucleotide sequences and antisense oligonucleotides that target (e.g., hybridize to) SOS1 or SOS2 mRNA are described in WO 2022/226377, U.S. provisional application No. 63/358,588, U.S. provisional application No. 63/476,895, and U.S. provisional application No. 63/479,103, the contents of which are hereby incorporated by reference in their entirety, and such nucleotide sequences and antisense oligonucleotides may be used in connection with this disclosure. Exemplary nucleotide sequences for constructing antisense oligonucleotides are provided herein in Table 13.

In various embodiments, the antisense oligonucleotide comprises at least 8, or at least 10, or at least 12 contiguous nucleotides of a nucleotide sequence from Table 13. In various embodiments, the antisense oligonucleotide has from 13 to 24 linked nucleotides, from 13 to 18 linked nucleotides, or from 13 to 16 linked nucleotides. In some embodiments, the antisense oligonucleotides are from 14 to 16 nucleotides in length. In some embodiments, the antisense oligonucleotide consists of a nucleotide sequence selected from Table 13. In some embodiments, the antisense oligonucleotide comprises or consists of a nucleotide sequence from Table 13. In some embodiments, the nucleotide sequences targeted SOS1 and/or SOS2 mRNA are constructed as gapmers as described herein. Unless otherwise indicated, the ASO compounds of Table 13 are constructed as gapmers with 2 or 3 LNA on each termini (and are fully phosphorothioate linked). In some embodiments, the wing segments may comprise a 5-methyl cytosine nucleobase, replacing an RNA nucleotide. In exemplary embodiments, the antisense oligonucleotide consists of the nucleotide sequence of SEQ ID NO: 582, SEQ ID NO: 835, or SEQ ID NO: 1000.

In some embodiments, the binding (e.g., hybridization) of the antisense oligonucleotide to the target mRNA leads to degradation of the target mRNA or blocks translation of the target mRNA. In some embodiments, the binding of the antisense oligonucleotide to the target mRNA creates a duplex nucleic acid molecule, which then recruits an endogenous nuclease for degradation of the mRNA. In some embodiments, the antisense oligonucleotide has a stretch of DNA nucleotides sufficient to recruit RNaseH, and thereby trigger degradation of the target mRNA. For example, the antisense oligonucleotide may have a stretch (e.g., a central stretch) of at least 6 or at least 8 DNA nucleotides, and which is optionally a stretch of 9 or 10 DNA nucleotides. In some embodiments, one or more DNA nucleotides comprise a 2′ chemical modification independently selected from 2′-Fluoro, 2′-Methyl, and 2′-Ethyl. For example, the antisense oligonucleotide may be a gapmer having a 5′ and a 3′ segment, each of the 5′ and 3′ segments being from 2 to 6 nucleotides or from 2 to 4 nucleotides, and where the 5′ and 3′ segments do not contain DNA nucleotides. In some embodiments, the gapmer is a 3-X-3 gapmers, with 3 RNA nucleotides (e.g., such as LNA) on each termini. In some embodiments, the gapmer is a 3-8-3 gapmer, having a central bock of DNA nucleotides and 5′ and 3′ segments of 3 RNA nucleotides each. In other embodiments, the gapmer is a 2-X-2 gapmer, such as a 2-10-2 gapmer or 2-9-2 gapmer, having a central block of 8-10 DNA nucleotides and a 5′ and 3′ segments of 2 RNA nucleotides each. In some embodiments, the gapmer is a 3-X-2 gapmer or a 2-X-3 gapmer. In some embodiments, the gapmer is a 3-10-3 gapmer. In some embodiments, one or more nucleotides of the 5′ segment and the 3′ segment comprise 2′-O substituents, optionally where all of the nucleotides of the 5′ segment and the 3′ segment comprise 2′-O substituents. Exemplary 2′-O substituents are independently selected from 2′-O methyl, 2′-O ethyl, 2′-O methoxyethyl (MOE), and a bridged nucleotide (e.g., a locked or bi-cyclic nucleotide) having a 2′ to 4′ bridge. In some embodiments, the bridged nucleotide has a methylene bridge (LNA) or is a constrained ethyl bridge (cEt). In still other embodiments, one or more cytosine nucleotides in the 5′ and 3′ segments may be 5-methyl cytosine (“5Me”) nucleobases (instead of 2′-modified RNA), and which can be 5-methyl dC ((5Me)dC). Such constructs are still considered gapmers, where the (5Me)dC nucleotide is flanked on at least one side by an RNA nucleotide.

The term “gapmer” refers to an oligonucleotide having a central block of deoxynucleotides (also referred to herein as “DNA nucleotides”) with 5′ and 3′ segments of RNA nucleotides. As used herein, the term “DNA nucleotide” refers to a nucleotide that is not an RNA nucleotide. DNA nucleotides typically have a 2′ H, but may alternatively have various 2′ chemical modifications, including 2′-halo and 2′-lower alkyl (e.g., C1-4). In some embodiments, the 2′ chemical modifications of DNA nucleotides are independently selected from 2′-Fluoro, 2′-Methyl, and 2′-Ethyl.

Locked nucleic acid (LNA) or “locked nucleotides” are described, for example, in U.S. Pat. Nos. 6,268,490; 6,316,198; 6,403,566; 6,770,748; 6,998,484; 6,670,461; and 7,034,133, all of which are hereby incorporated by reference in their entireties. LNAs are modified nucleotides that contain a bridge between the 2′ and 4′ carbons of the sugar moiety resulting in a “locked” conformation, and/or bicyclic structure. Other suitable locked nucleotides that can be incorporated in the oligonucleotides of this disclosure include those described in U.S. Pat. Nos. 6,403,566 and 6,833,361, both of which are hereby incorporated by reference in their entireties. In exemplary embodiments, the locked nucleotides are independently selected from a 2′ to 4′ methylene bridge (referred to as LNA) and a constrained ethyl (cEt) bridge (see U.S. Pat. Nos. 7,399,845 and 7,569,686, which are hereby incorporated by reference in their entireties).

In some embodiments, the antisense oligonucleotide has a modified backbone or modified internucleotide linkages. The term “internucleotide linkage” refers to the linkage between two adjacent nucleosides in a polynucleotide molecule. Naturally, the internucleotide linkage is a phosphodiester bond that forms between two oxygen atoms of the phosphate group and an oxygen atom of the sugar (either at 3′ or 5′ position) to form two ester bonds bridging between the two adjacent nucleosides. Modification of the internucleotide linkage may provide different characteristics, including but not limited to enhanced stability. For example, phosphorothioate or phosphorodithioate linkages increase the resistance of the internucleotide linkage to nucleases. Another example is phosphoacetate linkage (PACE), which improves transfection characteristics and enhances nuclease resistance. Internucleotide linkages and oligonucleotide backbone modifications which may be employed in the oligonucleotides of the present description include, but are not limited to, phosphodiester, phosphorothioate, phosphorodithioate, methylphosphonate, alkylphosphonate, alkylphosphonothioate, phosphotriester, phosphoramidate, phosphoramidite, phosphorodiamidate, siloxane, carbonate, carboalkoxy, acetamidate, carbamate, morpholino, peptide nucleic acid, borano, thioether, bridged phosphoramidate, bridged methylene phosphonate, bridged phosphorothioate, and sulfone internucleoside linkages. In some embodiments, the antisense oligonucleotide comprises one or more phosphorothioate or phosphorodithioate nucleotides.

In some embodiments, the antisense oligonucleotide comprises one or more phosphorothioate or phosphorodithioate internucleotide linkages. In some embodiments, phosphorothioate or phosphorodithioate bonds can be introduced between the last three to five nucleotides at the 5′- and/or 3′-end of the oligonucleotide to reduce exonuclease degradation. In some embodiments, the antisense oligonucleotide has a combination of phosphodiester and phosphorothioate/phosphorodithioate linkages. In some embodiments, the antisense oligonucleotide contains at least two, at least three, at least four, at least five, at least six, at least seven, at least eight, at least nine, or at least ten phosphorothioate or phosphorodithioate internucleotide linkages. In some embodiments, the antisense oligonucleotide comprises substantially alternating phosphodiester and phosphorothioate internucleotide linkages. In some embodiments, the antisense oligonucleotide is fully phosphorothioate/phosphorodithioate linked (i.e., all bonds are either phosphorothioate or phosphorodithioate). In some embodiments, the antisense oligonucleotide and the aptamer are fully phosphorothioate or phosphorodithioate linked.

In some embodiments, particularly where RNaseH recruitment is not desired, the antisense oligonucleotide has a morpholino backbone. Morpholino oligonucleotides do not generally trigger the degradation of their target RNA molecules, and can be effective for steric blocking of a target RNA sequence. Morpholino oligonucleotides and their synthesis are disclosed generally in U.S. Pat. Nos. 11,028,386, 10,947,533, and 10,927,378, each of which is hereby incorporated by reference in its entirety. In some embodiments, the antisense oligonucleotide comprises thiomorpholino nucleotides and/or other substituted or modified nucleotides such as those described, for example, in WO/2019/060522 and WO/2018/057430, each of which is hereby incorporated by reference in its entirety. For example, Langner et al. describe methods for synthesizing oligonucleotide analogs dubbed thiophosphoramidate morpholino oligonucleotides (TMOs) which incorporate morpholino nucleosides and phosphorothioate linkages (“Synthesis and characterization of thiophosphoramidate morpholino oligonucleotides and chimeras.” JACS 142.38 (2020): 16240-16253; see also Dumbović, Gabrijela, et al. “Nuclear compartmentalization of TERT mRNA and TUGI lncRNA is driven by intron retention.” Nature Communications 12.1 (2021): 1-19; both of which are hereby incorporated by reference in their entireties). Thus, the antisense oligonucleotides described herein may comprise full or partial TMO-modified nucleotides, or may comprise chimeras of TMO-modified nucleotides and unmodified nucleotides and/or other nucleotides comprising different modifications (e.g., LNAs).

In some embodiments, the antisense oligonucleotide may contain one or more modified bases. In some embodiments, cytosine is replaced with 5-methylcytosine, which may enhance base pairing. Other modified bases (particularly of cytosine or guanine) can be employed to reduce immunogenicity, where needed. Other modified bases are described in U.S. Pat. No. 10,064,959, which is hereby incorporated by reference.

In embodiments, the melting temperature of the antisense oligonucleotide hybridized to its target sequence is at least about 35° C. The Tm of an oligonucleotide is the temperature at which 50% of the oligonucleotide is duplexed with its perfect complement and 50% is free in solution. The Tm can be determined experimentally by measuring the absorbance change of the oligonucleotide with its complement as a function of temperature. The Tm can also be estimated using known publicly available Tm calculators. In some embodiments, the Tm of the oligonucleotide hybridized to its target sequence is at least about 40° C., or at least about 45° C., or at least about 50° C. In some embodiments, the Tm of the oligonucleotide hybridized to its target sequence is from about 35° C. to about 60° C. In some embodiments, the Tm of the oligonucleotide hybridized to its target sequence is from about 40° C. to about 60° C., or from about 50° C. to about 60° C.

In various embodiments, the aptamer and the antisense oligonucleotide are linked directly or indirectly through a linker. In some embodiments, the antisense oligonucleotide is conjugated directly or indirectly to the 3′ end or the 5′ end of the aptamer. In embodiments, the aptamer is linked directly or indirectly to the 3′ end of the aptamer. When conjugated indirectly through a linker, the linker may or may not be biologically cleavable (e.g., by a nuclease or other enzyme, or by disulfide reduction). Exemplary linkers can comprise amine, ester, and/or disulfide functionalities. Disulfide-containing linkers can be reduced by glutathione in endocytic vesicles, for example, to de-couple the aptamer and antisense oligonucleotide. In some embodiments, the linker comprises an alkylene (e.g., C2 to C12, or C2 to C8, or C2 to C6) or oligoethylene glycol spacer, providing, for example, 3 to 60 atom spacers, such as 3, 6, 9, 12, 15, or 18 atom spacers. An exemplary Spacer 18, also referred to here as HEG spacer (hexaethylene glycol), is an 18-atom spacer that can be placed at 5′, 3′ or internally. Spacer 18 can be incorporated in consecutive additions whenever a longer spacer is required (e.g., Sp18-Sp18).

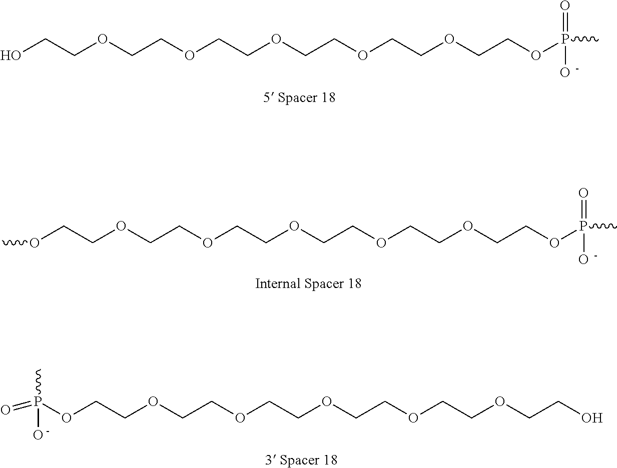

In some embodiments, the linker is a C3 propyl spacer. Similar to the Spacer 18, multiple C3 spacers can be added to introduce a longer spacer arm (e.g., {SpC3}{SpC3}).

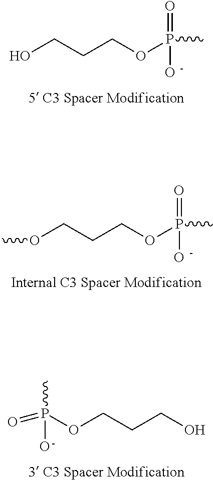

In some embodiments, the aptamer and the antisense oligonucleotide are linked directly through an oligonucleotide linker, for example, of from 1 to 30 nucleotides, 1 to 20 nucleotides, or from 2 to 15 nucleotides, or from 6 to 15 nucleotides, or from 8 to 14 nucleotides. In some embodiments, the linker is 3 nucleotides. In some embodiments, the linker is 4 nucleotides. In some embodiments, the linker is 5 nucleotides. In some embodiments, the linker is 6 nucleotides. In some embodiments, the linker is 7 nucleotides. In some embodiments, the linker is 8 nucleotides. In some embodiments, the linker is 9 nucleotides. In some embodiments, the linker is 10 nucleotides. In some embodiments, the linker is 11 nucleotides. In some embodiments, the linker is 12 nucleotides. The nucleotide composition or sequence of the linker can be selected to avoid formation of secondary structures. For example, the linker may substantially avoid combinations of nucleotides that form base pairs, such as Watson-Crick base pairs, as well as G and U nucleotides. In some embodiments, the nucleotide linker is a stretch of a single nucleobase, such as oligoA (e.g., an A8 to A14 linker, such as an A12 linker). In various embodiments, the linker comprises DNA or RNA nucleotides. For example, in some embodiments the linker comprises DNA nucleotides, and does not contain a modified backbone. In some embodiments, the linker comprises one or more RNA nucleotides to allow for RNase cleavage.

In some embodiments, the composition further comprises a sterol conjugate (e.g., cholesterol conjugate) or fatty acid conjugate such as a palmitoyl or stearyl lipid conjugate, which is optionally conjugated to the 3′ end of an aptamer-antisense oligonucleotide conjugate. These moieties can enhance cell penetration. See U.S. Pat. No. 9,012,225, which is hereby incorporated by reference in its entirety.

In some embodiments, the antisense oligonucleotide is encapsulated in a particle, and the aptamer is presented on the surface of the particle. In various embodiments, the particle is a liposome, polymeric nanoparticle, or lipid nanoparticle (LNP). Exemplary polymeric nanoparticles can be formed of PLA, PLGA, or PEG copolymers thereof. In some embodiments, the particle comprises poly(β amino ester) polymers. In various embodiments, the LNPs comprise a cationic or ionizable lipid, a neutral lipid, a cholesterol or cholesterol moiety, and a PEGylated lipid. In exemplary embodiments, the aptamer is conjugated to the termini of a portion of PEG groups that form a hydrophilic outer sheath.

In some embodiments, the lipid nanoparticle (or LNP) comprises a structural lipid. Exemplary structural lipids can be selected from one or more of cholesterol, fecosterol, sitosterol, ergosterol, campesterol, stigmasterol, brassicasterol, tomatidine, ursolic acid, and tocopherols (e.g., alpha tocopherol). In some embodiments, the structural lipid is cholesterol.

In some embodiments, the LNP comprises one or more phospholipids. Exemplary phospholipids are selected from the group consisting of cardiolipins, sterol modified lipids (modified with a cholesterol moiety attached at the sn-2 carbon of the glycerol backbone), mixed-acyl glycerophospholipids, and symmetrical acyl glycerophospholipids. Head groups for acyl glycerophospholipids include, for example, phosphatidic acid, lysophosphatidic acid, phosphatidylcholine, phosphatidylethanolamine, phosphatidylglycerol, phosphoinositides, and phosphatidylserine. Exemplary phospholipids are selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanol amine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle composition further comprises one or more PEG lipids. A PEG lipid is a lipid modified with polyethylene glycol. Exemplary PEG lipids are selected from one or more of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol. A PEG lipid may be selected from PEG-c-DOMG, PEG-DMG, PEG-DLPE, PEG-DMPE, PEG-DPPC, PEG-Cholesterol, PEG tocopherol, or a PEG-DSPE lipid.

Lipid particle formulations that find use with embodiments of the present disclosure include those described in U.S. Pat. Nos. 9,738,593; 10,221,127; 10,166,298, which are hereby incorporated by reference in their entirety. In some embodiments, the liposomes or nanoparticles further comprise a targeting moiety as described.

In another aspect, the present disclosure provides a method for treating a subject having pancreatic cancer. In various embodiments, the cancer is adenocarcinoma (e.g., pancreatic ductal adenocarcinoma), squamous cell carcinoma, adenosquamous carcinoma, and colloid carcinoma. In some embodiments, the pancreatic cancer is Stage 1 cancer, Stage 2 cancer, Stage 3 cancer, or Stage 4 cancer. In some embodiments, the cancer is non-metastatic. In other embodiments, the cancer is metastatic. The method comprises administering an effective amount of the composition described herein to the subject. In some embodiments, the subject is identified as having pancreatic cancer with a KRAS mutation, such as a KRAS mutation selected from G12C mutation, G12V mutation, G12A mutation, and G12D mutation. In such embodiments, a composition can be selected for specificity to the mutant KRAS mRNA, as already described (see Tables 3 and 4, for example).

In various embodiments, the compositions of the present disclosure are administered parenterally, such as by intravenous or intra-arterial infusion. In some embodiments, the compositions are administered intramuscularly, subcutaneously, or by direct infusion to target tissue (e.g., pancreatic tissue).

Dosing and administration schedules can vary, depending on the condition of the patient, and the chemistry of the compound. In various embodiments, the compositions are administered about weekly, about bimonthly (i.e., about every other week), about monthly, or about quarterly. Dosing and administration schedules can further include varying dosing and administration frequency based on the patient's response.

As used herein, the term “about”, unless the context requires otherwise, means±10% of an associated value.

Other aspects and embodiments of the present disclosure will be apparent from the following Examples.

TABLE 1
Exemplary antisense oligonucleotides (ASOs)
targeting KRAS Wild Type (WT) mRNA

| SEQ ID NO: | ASO sequence |
|---|---|
| SEQ ID NO: 1 | TCCTCTATTGTTGG |
| SEQ ID NO: 2 | ATCCTCTATTGTTG |
| SEQ ID NO: 3 | AATCCTCTATTGTT |
| SEQ ID NO: 4 | GAATCCTCTATTGT |
| SEQ ID NO: 5 | GGAATCCTCTATTG |
| SEQ ID NO: 6 | AGGAATCCTCTATT |
| SEQ ID NO: 7 | TAGGAATCCTCTAT |
| SEQ ID NO: 8 | GTAGGAATCCTCTA |
| SEQ ID NO: 9 | TGTAGGAATCCTCT |
| SEQ ID NO: 10 | CTGTAGGAATCCTC |
| SEQ ID NO: 11 | CCTGTAGGAATCCT |
| SEQ ID NO: 12 | TCCTGTAGGAATCC |
| SEQ ID NO: 13 | TTCCTGTAGGAATC |
| SEQ ID NO: 14 | CTTCCTGTAGGAAT |
| SEQ ID NO: 15 | GCTTCCTGTAGGAA |
| SEQ ID NO: 16 | TGCTTCCTGTAGGA |
| SEQ ID NO: 17 | TTGCTTCCTGTAGG |
| SEQ ID NO: 18 | CTTGCTTCCTGTAG |
| SEQ ID NO: 19 | ACTTGCTTCCTGTA |
| SEQ ID NO: 20 | TACTTGCTTCCTGT |
| SEQ ID NO: 21 | CTACTTGCTTCCTG |
| SEQ ID NO: 22 | ACTACTTGCTTCCT |
| SEQ ID NO: 23 | TACTACTTGCTTCC |
| SEQ ID NO: 24 | TTACTACTTGCTTC |
| SEQ ID NO: 25 | ATTACTACTTGCTT |
| SEQ ID NO: 26 | AATTACTACTTGCT |
| SEQ ID NO: 27 | CAATTACTACTTGC |
| SEQ ID NO: 28 | TCAATTACTACTTG |
| SEQ ID NO: 29 | ATCAATTACTACTT |
| SEQ ID NO: 30 | CATCAATTACTACT |
| SEQ ID NO: 31 | CCATCAATTACTAC |
| SEQ ID NO: 32 | TCCATCAATTACTA |
| SEQ ID NO: 33 | CTCCATCAATTACT |
| SEQ ID NO: 34 | TCTCCATCAATTAC |
| SEQ ID NO: 35 | TTCTCCATCAATTA |
| SEQ ID NO: 36 | TTTCTCCATCAATT |
| SEQ ID NO: 37 | GTTTCTCCATCAAT |
| SEQ ID NO: 38 | GGTTTCTCCATCAA |
| SEQ ID NO: 39 | AGGTTTCTCCATCA |
| SEQ ID NO: 40 | CAGGTTTCTCCATC |
| SEQ ID NO: 41 | ACAGGTTTCTCCAT |
| SEQ ID NO: 42 | GACAGGTTTCTCCA |
| SEQ ID NO: 43 | AGACAGGTTTCTCC |
| SEQ ID NO: 44 | GAGACAGGTTTCTC |
| SEQ ID NO: 45 | AGAGACAGGTTTCT |
| SEQ ID NO: 46 | AAGAGACAGGTTTC |
| SEQ ID NO: 47 | CAAGAGACAGGTTT |
| SEQ ID NO: 48 | CCAAGAGACAGGTT |
| SEQ ID NO: 49 | TCCAAGAGACAGGT |
| SEQ ID NO: 50 | ATCCAAGAGACAGG |
| SEQ ID NO: 51 | TATCCAAGAGACAG |
| SEQ ID NO: 52 | ATATCCAAGAGACA |
| SEQ ID NO: 53 | AATATCCAAGAGAC |
| SEQ ID NO: 54 | GAATATCCAAGAGA |
| SEQ ID NO: 55 | AGAATATCCAAGAG |
| SEQ ID NO: 56 | GAGAATATCCAAGA |
| SEQ ID NO: 57 | CGAGAATATCCAAG |
| SEQ ID NO: 58 | TCGAGAATATCCAA |
| SEQ ID NO: 59 | GTCGAGAATATCCA |
| SEQ ID NO: 60 | TGTCGAGAATATCC |
| SEQ ID NO: 61 | GTGTCGAGAATATC |
| SEQ ID NO: 62 | TGTGTCGAGAATAT |
| SEQ ID NO: 63 | CTGTGTCGAGAATA |
| SEQ ID NO: 64 | GCTGTGTCGAGAAT |
| SEQ ID NO: 65 | TGCTGTGTCGAGAA |
| SEQ ID NO: 66 | CTGCTGTGTCGAGA |
| SEQ ID NO: 67 | CCTGCTGTGTCGAG |
| SEQ ID NO: 68 | ACCTGCTGTGTCGA |
| SEQ ID NO: 69 | GACCTGCTGTGTCG |
| SEQ ID NO: 70 | TGACCTGCTGTGTC |
| SEQ ID NO: 71 | TTGACCTGCTGTGT |
| SEQ ID NO: 72 | CTTGACCTGCTGTG |
| SEQ ID NO: 73 | TCTTGACCTGCTGT |
| SEQ ID NO: 74 | CTCTTGACCTGCTG |
| SEQ ID NO: 75 | CCTCTTGACCTGCT |
| SEQ ID NO: 76 | TCCTCTTGACCTGC |
| SEQ ID NO: 77 | CTCCTCTTGACCTG |
| SEQ ID NO: 78 | ACTCCTCTTGACCT |
| SEQ ID NO: 79 | TACTCCTCTTGACC |
| SEQ ID NO: 80 | GTACTCCTCTTGAC |
| SEQ ID NO: 81 | TGTACTCCTCTTGA |
| SEQ ID NO: 82 | CTGTACTCCTCTTG |
| SEQ ID NO: 83 | ACTGTACTCCTCTT |
| SEQ ID NO: 84 | CACTGTACTCCTCT |
| SEQ ID NO: 85 | GCACTGTACTCCTC |
| SEQ ID NO: 86 | TGCACTGTACTCCT |
| SEQ ID NO: 87 | TTGCACTGTACTCC |
| SEQ ID NO: 88 | ATTGCACTGTACTC |
| SEQ ID NO: 89 | CATTGCACTGTACT |
| SEQ ID NO: 90 | TCATTGCACTGTAC |
| SEQ ID NO: 91 | CTCATTGCACTGTA |
| SEQ ID NO: 92 | CCTCATTGCACTGT |
| SEQ ID NO: 93 | CCCTCATTGCACTG |
| SEQ ID NO: 94 | TCCCTCATTGCACT |
| SEQ ID NO: 95 | GTCCCTCATTGCAC |
| SEQ ID NO: 96 | GGTCCCTCATTGCA |
| SEQ ID NO: 97 | TGGTCCCTCATTGC |
| SEQ ID NO: 98 | CTGGTCCCTCATTG |
| SEQ ID NO: 99 | ACTGGTCCCTCATT |
| SEQ ID NO: 100 | TACTGGTCCCTCAT |
| SEQ ID NO: 101 | GTACTGGTCCCTCA |
| SEQ ID NO: 102 | TGTACTGGTCCCTC |
| SEQ ID NO: 103 | ATGTACTGGTCCCT |
| SEQ ID NO: 104 | CATGTACTGGTCCC |
| SEQ ID NO: 105 | TCATGTACTGGTCC |
| SEQ ID NO: 106 | CTCATGTACTGGTC |
| SEQ ID NO: 107 | CCTCATGTACTGGT |
| SEQ ID NO: 108 | TCCTCATGTACTGG |
| SEQ ID NO: 109 | GTCCTCATGTACTG |
| SEQ ID NO: 110 | AGTCCTCATGTACT |
| SEQ ID NO: 111 | CAGTCCTCATGTAC |
| SEQ ID NO: 112 | CCAGTCCTCATGTA |
| SEQ ID NO: 113 | CCCAGTCCTCATGT |
| SEQ ID NO: 114 | CCCCAGTCCTCATG |
| SEQ ID NO: 115 | TCCCCAGTCCTCAT |
| SEQ ID NO: 116 | CTCCCCAGTCCTCA |
| SEQ ID NO: 117 | CCTCCCCAGTCCTC |
| SEQ ID NO: 118 | CCCTCCCCAGTCCT |
| SEQ ID NO: 119 | GCCCTCCCCAGTCC |
| SEQ ID NO: 120 | AGCCCTCCCCAGTC |
| SEQ ID NO: 121 | AAGCCCTCCCCAGT |
| SEQ ID NO: 122 | AAAGCCCTCCCCAG |
| SEQ ID NO: 123 | GAAAGCCCTCCCCA |
| SEQ ID NO: 124 | AGAAAGCCCTCCCC |
| SEQ ID NO: 125 | AAGAAAGCCCTCCC |
| SEQ ID NO: 126 | AAAGAAAGCCCTCC |
| SEQ ID NO: 127 | CAAAGAAAGCCCTC |
| SEQ ID NO: 128 | ACAAAGAAAGCCCT |
| SEQ ID NO: 129 | CACAAAGAAAGCCC |
| SEQ ID NO: 130 | ACACAAAGAAAGCC |
| SEQ ID NO: 131 | TACACAAAGAAAGC |
| SEQ ID NO: 132 | ATACACAAAGAAAG |
| SEQ ID NO: 133 | AATACACAAAGAAA |
| SEQ ID NO: 134 | AAATACACAAAGAA |
| SEQ ID NO: 135 | CAAATACACAAAGA |
| SEQ ID NO: 136 | GCAAATACACAAAG |
| SEQ ID NO: 137 | GGCAAATACACAAA |
| SEQ ID NO: 138 | TGGCAAATACACAA |
| SEQ ID NO: 139 | ATGGCAAATACACA |
| SEQ ID NO: 140 | TATGGCAAATACAC |
| SEQ ID NO: 141 | TTATGGCAAATACA |
| SEQ ID NO: 142 | TTTATGGCAAATAC |
| SEQ ID NO: 143 | ATTTATGGCAAATA |
| SEQ ID NO: 144 | TATTTATGGCAAAT |
| SEQ ID NO: 145 | TTATTTATGGCAAA |
| SEQ ID NO: 146 | ATTATTTATGGCAA |
| SEQ ID NO: 147 | TATTATTTATGGCA |
| SEQ ID NO: 148 | GTATTATTTATGGC |
| SEQ ID NO: 149 | AGTATTATTTATGG |
| SEQ ID NO: 150 | TAGTATTATTTATG |
| SEQ ID NO: 151 | TTAGTATTATTTAT |
| SEQ ID NO: 152 | TTTAGTATTATTTA |
| SEQ ID NO: 153 | ATTTAGTATTATTT |
| SEQ ID NO: 154 | GATTTAGTATTATT |
| SEQ ID NO: 155 | TGATTTAGTATTAT |
| SEQ ID NO: 156 | ATGATTTAGTATTA |
| SEQ ID NO: 157 | AATGATTTAGTATT |
| SEQ ID NO: 158 | AAATGATTTAGTAT |
| SEQ ID NO: 159 | CAAATGATTTAGTA |
| SEQ ID NO: 160 | TCAAATGATTTAGT |
| SEQ ID NO: 161 | TTCAAATGATTTAG |
| SEQ ID NO: 162 | CTTCAAATGATTTA |
| SEQ ID NO: 163 | TCTTCAAATGATTT |
| SEQ ID NO: 164 | ATCTTCAAATGATT |
| SEQ ID NO: 165 | TATCTTCAAATGAT |
| SEQ ID NO: 166 | ATATCTTCAAATGA |
| SEQ ID NO: 167 | AATATCTTCAAATG |
| SEQ ID NO: 168 | GAATATCTTCAAAT |
| SEQ ID NO: 169 | TGAATATCTTCAAA |
| SEQ ID NO: 170 | GTGAATATCTTCAA |
| SEQ ID NO: 171 | GGTGAATATCTTCA |
| SEQ ID NO: 172 | TGGTGAATATCTTC |
| SEQ ID NO: 173 | ATGGTGAATATCTT |
| SEQ ID NO: 174 | AATGGTGAATATCT |
| SEQ ID NO: 175 | TAATGGTGAATATC |
| SEQ ID NO: 176 | ATAATGGTGAATAT |
| SEQ ID NO: 177 | TATAATGGTGAATA |
| SEQ ID NO: 178 | CTATAATGGTGAAT |
| SEQ ID NO: 179 | TCTATAATGGTGAA |
| SEQ ID NO: 180 | CTCTATAATGGTGA |
| SEQ ID NO: 181 | TCTCTATAATGGTG |
| SEQ ID NO: 182 | TTCTCTATAATGGT |
| SEQ ID NO: 183 | GTTCTCTATAATGG |
| SEQ ID NO: 184 | TGTTCTCTATAATG |
| SEQ ID NO: 185 | TTGTTCTCTATAAT |
| SEQ ID NO: 186 | TTTGTTCTCTATAA |
| SEQ ID NO: 187 | ATTTGTTCTCTATA |
| SEQ ID NO: 188 | AATTTGTTCTCTAT |
| SEQ ID NO: 189 | TAATTTGTTCTCTA |
| SEQ ID NO: 190 | TTAATTTGTTCTCT |
| SEQ ID NO: 191 | TTTAATTTGTTCTC |
| SEQ ID NO: 192 | TTTTAATTTGTTCT |
| SEQ ID NO: 193 | CTTTTAATTTGTTC |
| SEQ ID NO: 194 | TCTTTTAATTTGTT |
| SEQ ID NO: 195 | CTCTTTTAATTTGT |
| SEQ ID NO: 196 | ACTCTTTTAATTTG |
| SEQ ID NO: 197 | AACTCTTTTAATTT |
| SEQ ID NO: 198 | TAACTCTTTTAATT |
| SEQ ID NO: 199 | TTAACTCTTTTAAT |
| SEQ ID NO: 200 | CTTAACTCTTTTAA |
| SEQ ID NO: 201 | CCTTAACTCTTTTA |
| SEQ ID NO: 202 | TCCTTAACTCTTTT |
| SEQ ID NO: 203 | GTCCTTAACTCTTT |
| SEQ ID NO: 204 | AGTCCTTAACTCTT |
| SEQ ID NO: 205 | GAGTCCTTAACTCT |
| SEQ ID NO: 206 | AGAGTCCTTAACTC |
| SEQ ID NO: 207 | CAGAGTCCTTAACT |
| SEQ ID NO: 208 | TCAGAGTCCTTAAC |
| SEQ ID NO: 209 | TTCAGAGTCCTTAA |
| SEQ ID NO: 210 | CTTCAGAGTCCTTA |
| SEQ ID NO: 211 | TCTTCAGAGTCCTT |
| SEQ ID NO: 212 | ATCTTCAGAGTCCT |
| SEQ ID NO: 213 | CATCTTCAGAGTCC |
| SEQ ID NO: 214 | ACATCTTCAGAGTC |
| SEQ ID NO: 215 | TACATCTTCAGAGT |
| SEQ ID NO: 216 | GTACATCTTCAGAG |
| SEQ ID NO: 217 | GGTACATCTTCAGA |
| SEQ ID NO: 218 | AGGTACATCTTCAG |
| SEQ ID NO: 219 | TAGGTACATCTTCA |
| SEQ ID NO: 220 | ATAGGTACATCTTC |
| SEQ ID NO: 221 | CATAGGTACATCTT |
| SEQ ID NO: 222 | CCATAGGTACATCT |
| SEQ ID NO: 223 | ACCATAGGTACATC |
| SEQ ID NO: 224 | GACCATAGGTACAT |
| SEQ ID NO: 225 | GGACCATAGGTACA |
| SEQ ID NO: 226 | AGGACCATAGGTAC |
| SEQ ID NO: 227 | TAGGACCATAGGTA |
| SEQ ID NO: 228 | CTAGGACCATAGGT |
| SEQ ID NO: 229 | ACTAGGACCATAGG |
| SEQ ID NO: 230 | TACTAGGACCATAG |
| SEQ ID NO: 231 | CTACTAGGACCATA |
| SEQ ID NO: 232 | CCTACTAGGACCAT |
| SEQ ID NO: 233 | TCCTACTAGGACCA |
| SEQ ID NO: 234 | TTCCTACTAGGACC |
| SEQ ID NO: 235 | TTTCCTACTAGGAC |
| SEQ ID NO: 236 | ATTTCCTACTAGGA |
| SEQ ID NO: 237 | TATTTCCTACTAGG |
| SEQ ID NO: 238 | TTATTTCCTACTAG |
| SEQ ID NO: 239 | TTTATTTCCTACTA |
| SEQ ID NO: 240 | ATTTATTTCCTACT |
| SEQ ID NO: 241 | CATTTATTTCCTAC |
| SEQ ID NO: 242 | ACATTTATTTCCTA |
| SEQ ID NO: 243 | CACATTTATTTCCT |
| SEQ ID NO: 244 | TCACATTTATTTCC |
| SEQ ID NO: 245 | ATCACATTTATTTC |
| SEQ ID NO: 246 | AATCACATTTATTT |
| SEQ ID NO: 247 | AAATCACATTTATT |
| SEQ ID NO: 248 | CAAATCACATTTAT |
| SEQ ID NO: 249 | GCAAATCACATTTA |
| SEQ ID NO: 250 | GGCAAATCACATTT |
| SEQ ID NO: 251 | AGGCAAATCACATT |
| SEQ ID NO: 252 | AAGGCAAATCACAT |
| SEQ ID NO: 253 | GAAGGCAAATCACA |
| SEQ ID NO: 254 | AGAAGGCAAATCAC |
| SEQ ID NO: 255 | TAGAAGGCAAATCA |
| SEQ ID NO: 256 | CTAGAAGGCAAATC |
| SEQ ID NO: 257 | TCTAGAAGGCAAAT |
| SEQ ID NO: 258 | TTCTAGAAGGCAAA |
| SEQ ID NO: 259 | GTTCTAGAAGGCAA |
| SEQ ID NO: 260 | TGTTCTAGAAGGCA |
| SEQ ID NO: 261 | CTGTTCTAGAAGGC |
| SEQ ID NO: 262 | ACTGTTCTAGAAGG |
| SEQ ID NO: 263 | TACTGTTCTAGAAG |
| SEQ ID NO: 264 | CTACTGTTCTAGAA |
| SEQ ID NO: 265 | TCTACTGTTCTAGA |
| SEQ ID NO: 266 | GTCTACTGTTCTAG |
| SEQ ID NO: 267 | TGTCTACTGTTCTA |
| SEQ ID NO: 268 | GTGTCTACTGTTCT |
| SEQ ID NO: 269 | TGTGTCTACTGTTC |
| SEQ ID NO: 270 | TTGTGTCTACTGTT |
| SEQ ID NO: 271 | TTTGTGTCTACTGT |
| SEQ ID NO: 272 | TTTTGTGTCTACTG |
| SEQ ID NO: 273 | GTTTTGTGTCTACT |
| SEQ ID NO: 274 | TGTTTTGTGTCTAC |
| SEQ ID NO: 275 | CTGTTTTGTGTCTA |
| SEQ ID NO: 276 | CCTGTTTTGTGTCT |
| SEQ ID NO: 277 | GCCTGTTTTGTGTC |
| SEQ ID NO: 278 | AGCCTGTTTTGTGT |
| SEQ ID NO: 279 | GAGCCTGTTTTGTG |
| SEQ ID NO: 280 | TGAGCCTGTTTTGT |
| SEQ ID NO: 281 | CTGAGCCTGTTTTG |
| SEQ ID NO: 282 | CCTGAGCCTGTTTT |
| SEQ ID NO: 283 | TCCTGAGCCTGTTT |
| SEQ ID NO: 284 | GTCCTGAGCCTGTT |
| SEQ ID NO: 285 | AGTCCTGAGCCTGT |
| SEQ ID NO: 286 | AAGTCCTGAGCCTG |
| SEQ ID NO: 287 | TAAGTCCTGAGCCT |
| SEQ ID NO: 288 | CTAAGTCCTGAGCC |
| SEQ ID NO: 289 | GCTAAGTCCTGAGC |
| SEQ ID NO: 290 | TGCTAAGTCCTGAG |
| SEQ ID NO: 291 | TTGCTAAGTCCTGA |
| SEQ ID NO: 292 | CTTGCTAAGTCCTG |
| SEQ ID NO: 293 | TCTTGCTAAGTCCT |
| SEQ ID NO: 294 | TTCTTGCTAAGTCC |
| SEQ ID NO: 295 | CTTCTTGCTAAGTC |
| SEQ ID NO: 296 | ACTTCTTGCTAAGT |
| SEQ ID NO: 297 | AACTTCTTGCTAAG |
| SEQ ID NO: 298 | TAACTTCTTGCTAA |
| SEQ ID NO: 299 | ATAACTTCTTGCTA |
| SEQ ID NO: 300 | CATAACTTCTTGCT |
| SEQ ID NO: 301 | CCATAACTTCTTGC |
| SEQ ID NO: 302 | TCCATAACTTCTTG |
| SEQ ID NO: 303 | TTCCATAACTTCTT |
| SEQ ID NO: 304 | ATTCCATAACTTCT |
| SEQ ID NO: 305 | AATTCCATAACTTC |
| SEQ ID NO: 306 | GAATTCCATAACTT |
| SEQ ID NO: 307 | GGAATTCCATAACT |
| SEQ ID NO: 308 | AGGAATTCCATAAC |
| SEQ ID NO: 309 | AAGGAATTCCATAA |
| SEQ ID NO: 310 | AAAGGAATTCCATA |
| SEQ ID NO: 311 | AAAAGGAATTCCAT |
| SEQ ID NO: 312 | TAAAAGGAATTCCA |
| SEQ ID NO: 313 | ATAAAAGGAATTCC |
| SEQ ID NO: 314 | AATAAAAGGAATTC |
| SEQ ID NO: 315 | CAATAAAAGGAATT |
| SEQ ID NO: 316 | TCAATAAAAGGAAT |
| SEQ ID NO: 317 | TTCAATAAAAGGAA |
| SEQ ID NO: 318 | TTTCAATAAAAGGA |
| SEQ ID NO: 319 | GTTTCAATAAAAGG |
| SEQ ID NO: 320 | TGTTTCAATAAAAG |
| SEQ ID NO: 321 | ATGTTTCAATAAAA |
| SEQ ID NO: 322 | GATGTTTCAATAAA |
| SEQ ID NO: 323 | TGATGTTTCAATAA |
| SEQ ID NO: 324 | CTGATGTTTCAATA |
| SEQ ID NO: 325 | GCTGATGTTTCAAT |
| SEQ ID NO: 326 | TGCTGATGTTTCAA |
| SEQ ID NO: 327 | TTGCTGATGTTTCA |
| SEQ ID NO: 328 | TTTGCTGATGTTTC |
| SEQ ID NO: 329 | CTTTGCTGATGTTT |
| SEQ ID NO: 330 | TCTTTGCTGATGTT |
| SEQ ID NO: 331 | GTCTTTGCTGATGT |
| SEQ ID NO: 332 | TGTCTTTGCTGATG |
| SEQ ID NO: 333 | TTGTCTTTGCTGAT |
| SEQ ID NO: 334 | CTTGTCTTTGCTGA |
| SEQ ID NO: 335 | TCTTGTCTTTGCTG |
| SEQ ID NO: 336 | GTCTTGTCTTTGCT |
| SEQ ID NO: 337 | TGTCTTGTCTTTGC |
| SEQ ID NO: 338 | CTGTCTTGTCTTTG |
| SEQ ID NO: 339 | TCTGTCTTGTCTTT |
| SEQ ID NO: 340 | CTCTGTCTTGTCTT |
| SEQ ID NO: 341 | TCTCTGTCTTGTCT |
| SEQ ID NO: 342 | CTCTCTGTCTTGTC |
| SEQ ID NO: 343 | ACTCTCTGTCTTGT |
| SEQ ID NO: 344 | CACTCTCTGTCTTG |
| SEQ ID NO: 345 | CCACTCTCTGTCTT |
| SEQ ID NO: 346 | TCCACTCTCTGTCT |
| SEQ ID NO: 347 | CTCCACTCTCTGTC |
| SEQ ID NO: 348 | CCTCCACTCTCTGT |
| SEQ ID NO: 349 | TCCTCCACTCTCTG |
| SEQ ID NO: 350 | ATCCTCCACTCTCT |
| SEQ ID NO: 351 | CATCCTCCACTCTC |
| SEQ ID NO: 352 | GCATCCTCCACTCT |
| SEQ ID NO: 353 | AGCATCCTCCACTC |
| SEQ ID NO: 354 | AAGCATCCTCCACT |
| SEQ ID NO: 355 | AAAGCATCCTCCAC |
| SEQ ID NO: 356 | AAAAGCATCCTCCA |
| SEQ ID NO: 357 | AAAAAGCATCCTCC |
| SEQ ID NO: 358 | TAAAAAGCATCCTC |
| SEQ ID NO: 359 | ATAAAAAGCATCCT |
| SEQ ID NO: 360 | TATAAAAAGCATCC |
| SEQ ID NO: 361 | GTATAAAAAGCATC |
| SEQ ID NO: 362 | TGTATAAAAAGCAT |
| SEQ ID NO: 363 | ATGTATAAAAAGCA |
| SEQ ID NO: 364 | AATGTATAAAAAGC |
| SEQ ID NO: 365 | CAATGTATAAAAAG |
| SEQ ID NO: 366 | CCAATGTATAAAAA |
| SEQ ID NO: 367 | ACCAATGTATAAAA |
| SEQ ID NO: 368 | CACCAATGTATAAA |
| SEQ ID NO: 369 | TCACCAATGTATAA |
| SEQ ID NO: 370 | CTCACCAATGTATA |
| SEQ ID NO: 371 | TCTCACCAATGTAT |
| SEQ ID NO: 372 | CTCTCACCAATGTA |
| SEQ ID NO: 373 | TCTCTCACCAATGT |
| SEQ ID NO: 374 | CTCTCTCACCAATG |
| SEQ ID NO: 375 | TCTCTCTCACCAAT |
| SEQ ID NO: 376 | ATCTCTCTCACCAA |
| SEQ ID NO: 377 | GATCTCTCTCACCA |
| SEQ ID NO: 378 | GGATCTCTCTCACC |
| SEQ ID NO: 379 | CGGATCTCTCTCAC |
| SEQ ID NO: 380 | TCGGATCTCTCTCA |
| SEQ ID NO: 381 | GTCGGATCTCTCTC |
| SEQ ID NO: 382 | TGTCGGATCTCTCT |
| SEQ ID NO: 383 | TTGTCGGATCTCTC |
| SEQ ID NO: 384 | ATTGTCGGATCTCT |
| SEQ ID NO: 385 | TATTGTCGGATCTC |
| SEQ ID NO: 386 | GTATTGTCGGATCT |
| SEQ ID NO: 387 | TGTATTGTCGGATC |
| SEQ ID NO: 388 | CTGTATTGTCGGAT |
| SEQ ID NO: 389 | TCTGTATTGTCGGA |
| SEQ ID NO: 390 | ATCTGTATTGTCGG |
| SEQ ID NO: 391 | AATCTGTATTGTCG |
| SEQ ID NO: 392 | CAATCTGTATTGTC |
| SEQ ID NO: 393 | TCAATCTGTATTGT |
| SEQ ID NO: 394 | TTCAATCTGTATTG |
| SEQ ID NO: 395 | TTTCAATCTGTATT |
| SEQ ID NO: 396 | TTTTCAATCTGTAT |
| SEQ ID NO: 397 | TTTTTCAATCTGTA |
| SEQ ID NO: 398 | TTTTTTCAATCTGT |
| SEQ ID NO: 399 | TTTTTTTCAATCTG |
| SEQ ID NO: 400 | ATTTTTTTCAATCT |
| SEQ ID NO: 401 | GATTTTTTTCAATC |
| SEQ ID NO: 402 | TGATTTTTTTCAAT |
| SEQ ID NO: 403 | CTGATTTTTTTCAA |
| SEQ ID NO: 404 | GCTGATTTTTTTCA |
| SEQ ID NO: 405 | TGCTGATTTTTTTC |
| SEQ ID NO: 406 | TTGCTGATTTTTTT |
| SEQ ID NO: 407 | TTTGCTGATTTTTT |
| SEQ ID NO: 408 | CTTTGCTGATTTTT |
| SEQ ID NO: 409 | TCTTTGCTGATTTT |
| SEQ ID NO: 410 | TTCTTTGCTGATTT |
| SEQ ID NO: 411 | CTTCTTTGCTGATT |
| SEQ ID NO: 412 | TCTTCTTTGCTGAT |
| SEQ ID NO: 413 | TTCTTCTTTGCTGA |
| SEQ ID NO: 414 | TTTCTTCTTTGCTG |
| SEQ ID NO: 415 | TTTTCTTCTTTGCT |
| SEQ ID NO: 416 | CTTTTCTTCTTTGC |
| SEQ ID NO: 417 | TCTTTTCTTCTTTG |
| SEQ ID NO: 418 | GTCTTTTCTTCTTT |
| SEQ ID NO: 419 | AGTCTTTTCTTCTT |
| SEQ ID NO: 420 | GAGTCTTTTCTTCT |
| SEQ ID NO: 421 | GGAGTCTTTTCTTC |
| SEQ ID NO: 422 | AGGAGTCTTTTCTT |
| SEQ ID NO: 423 | CAGGAGTCTTTTCT |
| SEQ ID NO: 424 | CCAGGAGTCTTTTC |
| SEQ ID NO: 425 | GCCAGGAGTCTTTT |
| SEQ ID NO: 426 | AGCCAGGAGTCTTT |
| SEQ ID NO: 427 | CAGCCAGGAGTCTT |
| SEQ ID NO: 428 | ACAGCCAGGAGTCT |
| SEQ ID NO: 429 | CACAGCCAGGAGTC |
| SEQ ID NO: 430 | ACACAGCCAGGAGT |
| SEQ ID NO: 431 | CACACAGCCAGGAG |
| SEQ ID NO: 432 | TCACACAGCCAGGA |
| SEQ ID NO: 433 | TTCACACAGCCAGG |
| SEQ ID NO: 434 | TTTCACACAGCCAG |
| SEQ ID NO: 435 | TTTTCACACAGCCA |
| SEQ ID NO: 436 | ATTTTCACACAGCC |
| SEQ ID NO: 437 | AATTTTCACACAGC |
| SEQ ID NO: 438 | TAATTTTCACACAG |
| SEQ ID NO: 439 | TTAATTTTCACACA |
| SEQ ID NO: 440 | TTTAATTTTCACAC |
| SEQ ID NO: 441 | TTTTAATTTTCACA |
| SEQ ID NO: 442 | TTTTTAATTTTCAC |
| SEQ ID NO: 443 | TTTTTTAATTTTCA |
| SEQ ID NO: 444 | ATTTTTTAATTTTC |
| SEQ ID NO: 445 | CATTTTTTAATTTT |
| SEQ ID NO: 446 | GCATTTTTTAATTT |
| SEQ ID NO: 447 | TGCATTTTTTAATT |
| SEQ ID NO: 448 | ATGCATTTTTTAAT |
| SEQ ID NO: 449 | AATGCATTTTTTAA |
| SEQ ID NO: 450 | TAATGCATTTTTTA |
| SEQ ID NO: 451 | ATAATGCATTTTTT |
| SEQ ID NO: 452 | TATAATGCATTTTT |
| SEQ ID NO: 453 | TTATAATGCATTTT |
| SEQ ID NO: 454 | ATTATAATGCATTT |
| SEQ ID NO: 455 | CATTATAATGCATT |
| SEQ ID NO: 456 | ACATTATAATGCAT |
| SEQ ID NO: 457 | TACATTATAATGCA |
| SEQ ID NO: 458 | TATCGTCAAGGCACTC |
| SEQ ID NO: 459 | CCTCATTGCACTGTAC |
| SEQ ID NO: 460 | TATCCAAACTGCCCTA |

TABLE 2
Exemplary selected antisense oligonucleotides
(ASOs) targeting KRAS Wild Type (WT) mRNA

| SEQ ID NO: | ASO sequence |
|---|---|
| SEQ ID NO: 30 | CATCAATTACTACT |
| SEQ ID NO: 114 | CCCCAGTCCTCATG |
| SEQ ID NO: 115 | TCCCCAGTCCTCAT |
| SEQ ID NO: 116 | CTCCCCAGTCCTCA |
| SEQ ID NO: 117 | CCTCCCCAGTCCTC |
| SEQ ID NO: 118 | CCCTCCCCAGTCCT |
| SEQ ID NO: 151 | TTAGTATTATTTAT |
| SEQ ID NO: 152 | TTTAGTATTATTTA |
| SEQ ID NO: 153 | ATTTAGTATTATTT |
| SEQ ID NO: 154 | GATTTAGTATTATT |
| SEQ ID NO: 155 | TGATTTAGTATTAT |
| SEQ ID NO: 156 | ATGATTTAGTATTA |
| SEQ ID NO: 157 | AATGATTTAGTATT |
| SEQ ID NO: 293 | TCTTGCTAAGTCCT |
| SEQ ID NO: 294 | TTCTTGCTAAGTCC |
| SEQ ID NO: 295 | CTTCTTGCTAAGTC |
| SEQ ID NO: 300 | CATAACTTCTTGCT |
| SEQ ID NO: 301 | CCATAACTTCTTGC |
| SEQ ID NO: 302 | TCCATAACTTCTTG |
| SEQ ID NO: 303 | TTCCATAACTTCTT |
| SEQ ID NO: 328 | TTTGCTGATGTTTC |
| SEQ ID NO: 329 | CTTTGCTGATGTTT |
| SEQ ID NO: 330 | TCTTTGCTGATGTT |
| SEQ ID NO: 331 | GTCTTTGCTGATGT |
| SEQ ID NO: 334 | CTTGTCTTTGCTGA |
| SEQ ID NO: 335 | TCTTGTCTTTGCTG |
| SEQ ID NO: 336 | GTCTTGTCTTTGCT |
| SEQ ID NO: 337 | TGTCTTGTCTTTGC |
| SEQ ID NO: 338 | CTGTCTTGTCTTTG |
| SEQ ID NO: 339 | TCTGTCTTGTCTTT |
| SEQ ID NO: 340 | CTCTGTCTTGTCTT |
| SEQ ID NO: 341 | TCTCTGTCTTGTCT |
| SEQ ID NO: 342 | CTCTCTGTCTTGTC |
| SEQ ID NO: 395 | TTTCAATCTGTATT |
| SEQ ID NO: 396 | TTTTCAATCTGTAT |
| SEQ ID NO: 397 | TTTTTCAATCTGTA |
| SEQ ID NO: 398 | TTTTTTCAATCTGT |
| SEQ ID NO: 404 | GCTGATTTTTTTCA |
| SEQ ID NO: 405 | TGCTGATTTTTTTC |
| SEQ ID NO: 406 | TTGCTGATTTTTTT |
| SEQ ID NO: 407 | TTTGCTGATTTTTT |
| SEQ ID NO: 408 | CTTTGCTGATTTTT |
| SEQ ID NO: 409 | TCTTTGCTGATTTT |
| SEQ ID NO: 410 | TTCTTTGCTGATTT |
| SEQ ID NO: 411 | CTTCTTTGCTGATT |
| SEQ ID NO: 412 | TCTTCTTTGCTGAT |
| SEQ ID NO: 413 | TTCTTCTTTGCTGA |
| SEQ ID NO: 414 | TTTCTTCTTTGCTG |
| SEQ ID NO: 415 | TTTTCTTCTTTGCT |
| SEQ ID NO: 416 | CTTTTCTTCTTTGC |
| SEQ ID NO: 417 | TCTTTTCTTCTTTG |
| SEQ ID NO: 418 | GTCTTTTCTTCTTT |
| SEQ ID NO: 419 | AGTCTTTTCTTCTT |
| SEQ ID NO: 420 | GAGTCTTTTCTTCT |
| SEQ ID NO: 421 | GGAGTCTTTTCTTC |
| SEQ ID NO: 422 | AGGAGTCTTTTCTT |
| SEQ ID NO: 423 | CAGGAGTCTTTTCT |
| SEQ ID NO: 458 | TATCGTCAAGGCACTC |
| SEQ ID NO: 459 | CCTCATTGCACTGTAC |
| SEQ ID NO: 460 | TATCCAAACTGCCCTA |

TABLE 3
Exemplary antisense oligonucleotides
(ASOs) targeting KRAS mutated mRNA

| SEQ ID NO: | ASO sequence | ASO Number |
|---|---|---|
| SEQ ID NO: 461 | TGCCTACGCCACAAGC | 24 |
| SEQ ID NO: 462 | GCCTACGCCACAAGCT | 25 |
| SEQ ID NO: 463 | CCTACGCCACAAGCTC | 26 |
| SEQ ID NO: 464 | CTACGCCACAAGCTCC | 27 |
| SEQ ID NO: 465 | TACGCCACAAGCTCCA | 28 |
| SEQ ID NO: 466 | ACGCCACAAGCTCCAA | 29 |
| SEQ ID NO: 467 | CGCCACAAGCTCCAAC | 30 |
| SEQ ID NO: 468 | GCCACAAGCTCCAACT | 31 |
| SEQ ID NO: 469 | CCACAAGCTCCAACTA | 32 |
| SEQ ID NO: 470 | CACAAGCTCCAACTAC | 33 |
| SEQ ID NO: 471 | TTGCCTACGCCATCAG | 34 |
| SEQ ID NO: 472 | TGCCTACGCCATCAGC | 35 |
| SEQ ID NO: 473 | GCCTACGCCATCAGCT | 36 |
| SEQ ID NO: 474 | CCTACGCCATCAGCTC | 37 |
| SEQ ID NO: 475 | CTACGCCATCAGCTCC | 38 |
| SEQ ID NO: 476 | TACGCCATCAGCTCCA | 39 |
| SEQ ID NO: 477 | ACGCCATCAGCTCCAA | 40 |
| SEQ ID NO: 478 | CGCCATCAGCTCCAAC | 41 |
| SEQ ID NO: 479 | GCCATCAGCTCCAACT | 42 |
| SEQ ID NO: 480 | CCATCAGCTCCAACTA | 43 |
| SEQ ID NO: 481 | ACGCCACAAGCTCCA | 65 |
| SEQ ID NO: 482 | CGCCACAAGCTCCA | 66 |
| SEQ ID NO: 483 | ACGCCACAAGCTCC | 67 |
| SEQ ID NO: 484 | CTACGCCATCAGCTC | 68 |
| SEQ ID NO: 485 | CTACGCCATCAGCT | 69 |
| SEQ ID NO: 486 | TACGCCATCAGCTC | 70 |
| SEQ ID NO: 487 | ACGCCATCAGCTCC | 71 |
| SEQ ID NO: 488 | CCTACGCCAGCAGCTC | 72 |
| SEQ ID NO: 489 | CTACGCCAGCAGCTC | 73 |
| SEQ ID NO: 490 | CTACGCCAGCAGCT | 74 |
| SEQ ID NO: 491 | TACGCCAGCAGCTC | 75 |
| SEQ ID NO: 492 | ACGCCAGCAGCTCC | 76 |
| SEQ ID NO: 493 | CCTACGCCAACAGCTC | 77 |
| SEQ ID NO: 494 | CTACGCCAACAGCTC | 78 |
| SEQ ID NO: 495 | CTACGCCAACAGCT | 79 |
| SEQ ID NO: 496 | TACGCCAACAGCTC | 80 |
| SEQ ID NO: 497 | ACGCCAACAGCTCC | 81 |
| | | (2-10-2 |
| | | gapmer) |
| SEQ ID NO: 498 | CGCCACAAGCTCCA | 82 |
| SEQ ID NO: 499 | CGCCACAAGCTCC | 83 |
| SEQ ID NO: 500 | ACGCCATCAGCTC | 84 |
| SEQ ID NO: 501 | ACGCCAACAGCTC | 85 |
| SEQ ID NO: 502 | ACGCCAACAGCTCC | 86 |
| SEQ ID NO: 503 | CTACGCCAGCAGCTCC | 87 |
| SEQ ID NO: 504 | ACGCCAGCAGCTC | 88 |
| SEQ ID NO: 505 | CCTACGCAACCACG | 89 |
| SEQ ID NO: 506 | CTACGCAACCACGT | 90 |
| SEQ ID NO: 507 | CTACGCAACCACG | 91 |
| SEQ ID NO: 508 | GCCTACGTAACCAC | 92 |
| SEQ ID NO: 509 | CCTACGTAACCACG | 93 |
| SEQ ID NO: 510 | CCTACGTAACCAC | 94 |
| SEQ ID NO: 511 | +A(5Me) | 95 |
| | dC+GCCAACAG+C+T+C | “+” is LNA |
| SEQ ID NO: 512 | +A+C+GCCAACAGC+T+C | 96 |
| | (5Me)dC | “+” is LNA |
| SEQ ID NO: 513 | +A+C+GCCAACAGC+T | 97 |
| | (5Me)dC+C | “+” is LNA |
| SEQ ID NO: 514 | +A(5Me) | 98 |
| | dCGCCAACAGC+T+C | “+” is LNA |
| SEQ ID NO: 515 | +A+CGCCAACAG(5Me) | 99 |
| | dC+T+C | “+” is LNA |
| SEQ ID NO: 516 | +A+CGCCAACAG+C+T | 100 |
| | (5Me)dC | “+” is LNA |
| SEQ ID NO: 517 | +T+A(5Me) | 101 |
| | dCGCCAACAGC+T+C | “+” is LNA |
| SEQ ID NO: 518 | +T+A(5Me) | 102 |
| | dCGCCAACAG+C+T+C | “+” is LNA |
| SEQ ID NO: 519 | +T+A+CGCCAACAG+C+T | 103 |
| | (5Me)dC | “+” is LNA |
| SEQ ID NO: 520 | +T+A(5Me)dCG(5Me) | 104 |
| | dC(5Me)dCAA(5Me) | “+” is LNA |
| | dCAG(5Me)dC+T+C | |
| SEQ ID NO: 521 | +T+A(5Me)dCGCCAACAG | 105 |
| | (5Me)dC+T+C | “+” is LNA |
| SEQ ID NO: 522 | TACGCCAACAGCTC | 106 |
| | | 2-9-2 gapmer |
| SEQ ID NO: 523 | TACGCCAACAGCTC | 107 |
| | | 3-9-2 gapmer |
| SEQ ID NO: 524 | TACGCCAACAGCTC | 108 |
| | | 2-9-3 gapmer |

TABLE 4
Exemplary antisense oligonucleotides (ASOs)
targeting KRAS mRNA

| | Nucleic acid |
|---|---|
| SEQ ID NO | sequence |
| SEQ ID NO: 458 | TATCGTCAAGGCACTC |
| SEQ ID NO: 459 | CCTCATTGCACTGTAC |
| SEQ ID NO: 460 | TATCCAAACTGCCCTA |
| SEQ ID NO: 461 | TGCCTACGCCACAAGC |
| SEQ ID NO: 462 | GCCTACGCCACAAGCT |
| SEQ ID NO: 463 | CCTACGCCACAAGCTC |
| SEQ ID NO: 464 | CTACGCCACAAGCTCC |
| SEQ ID NO: 465 | TACGCCACAAGCTCCA |
| SEQ ID NO: 466 | ACGCCACAAGCTCCAA |
| SEQ ID NO: 467 | CGCCACAAGCTCCAAC |
| SEQ ID NO: 468 | GCCACAAGCTCCAACT |
| SEQ ID NO: 469 | CCACAAGCTCCAACTA |
| SEQ ID NO: 470 | CACAAGCTCCAACTAC |
| SEQ ID NO: 474 | CCTACGCCATCAGCTC |
| SEQ ID NO: 525 | GCTATTAGGAGTCTTT |
| SEQ ID NO: 482 | CGCCACAAGCTCCA |
| SEQ ID NO: 500 | ACGCCATCAGCTC |
| SEQ ID NO: 502 | ACGCCAACAGCTCC |
| SEQ ID NO: 504 | ACGCCAGCAGCTC |
| SEQ ID NO: 503 | CTACGCCAGCAGCTCC |
| SEQ ID NO: 481 | ACGCCACAAGCTCCA |
| SEQ ID NO: 482 | CGCCACAAGCTCCA |
| SEQ ID NO: 483 | ACGCCACAAGCTCC |
| SEQ ID NO: 484 | CTACGCCATCAGCTC |
| SEQ ID NO: 485 | CTACGCCATCAGCT |
| SEQ ID NO: 486 | TACGCCATCAGCTC |
| SEQ ID NO: 487 | ACGCCATCAGCTCC |
| SEQ ID NO: 488 | CCTACGCCAGCAGCTC |
| SEQ ID NO: 489 | CTACGCCAGCAGCTC |
| SEQ ID NO: 490 | CTACGCCAGCAGCT |
| SEQ ID NO: 491 | TACGCCAGCAGCTC |
| SEQ ID NO: 492 | ACGCCAGCAGCTCC |
| SEQ ID NO: 493 | CCTACGCCAACAGCTC |
| SEQ ID NO: 494 | CTACGCCAACAGCTC |
| SEQ ID NO: 495 | CTACGCCAACAGCT |
| SEQ ID NO: 496 | TACGCCAACAGCTC |
| SEQ ID NO: 497 | ACGCCAACAGCTCC |
| SEQ ID NO: 499 | CGCCACAAGCTCC |
| SEQ ID NO: 501 | ACGCCAACAGCTC |
| SEQ ID NO: 526 | TACGCCACAAGCTC |
| | (ASO 840) |
| SEQ ID NO: 527 | ACGCCACAAGCTC |
| SEQ ID NO: 528 | CTACGCCACAAGCT |
| SEQ ID NO: 529 | CTACGCCACAAGCTC |
| SEQ ID NO: 530 | ACGTCTATACACCA |
| SEQ ID NO: 531 | GAGCTGATGGCGTA |

### EXAMPLES

### Example 1: Knockdown of KRAS mRNA

### Cell Culture Condition and In Vitro Transfection

Various tumor cell lines with different KRAS mutations were plated at a density of 20,000 cells per well in a 96-well plates and were treated with both 5 nM and 20 nM of antisense oligonucleotide by transfection with Lipofectamine (Life Technology, USA). The transfection was conducted according to vendor's recommendation, with 0.3 μL Lipofectamine per well and incubated for 3 hours. After 2 days, cells were harvested and subjected to Quantigene assay for relative mRNA quantitation analysis (Life Technology, USA) and following the specification from vendor. The catalog numbers of KRAS and PPIB (reference gene to normalize the expression) probes are SA-50338 and SA-50155 respectively. The percent reduction of mRNA against a non-targeting control oligonucleotide were calculated and summarized in Table 5. The results show that KRAS-targeting oligonucleotides can inhibit KRAS mRNA in NCI-H358 cells (non-small cell lung cancer cell line that harbors a KRAS mutation).

TABLE 5
Knockdown of KRAS mRNA by antisense
oligonucleotides (3-X-3 LNA gapmers,
phosphorothioate-linked; nucleotide
sequences from Table 4)

| | | Antisense | | |
|---|---|---|---|---|
| | Target | Oligonucleotide | % | |
| Cell | KRAS | (ASO) | Inhibition | |
| lines | mRNA | Sequence | 20 nM | 5 nM |
| NCI- | Negative | CACGTCTATACACCAC | 0 | 0 |
| H358 | control | (SEQ ID NO: 532) | | |
| NCI- | Negative | TTTCTGAGGATTATCG | −9.3 | −18.2 |
| H358 | control | (SEQ ID NO: 533) | | |
| NCI- | hKRAS | TATCGTCAAGGCACTC | 21.1 | 10.8 |
| H358 | | (SEQ ID NO: 458) | | |
| NCI- | hKRAS | CCTCATTGCACTGTAC | 36.5 | 33.4 |
| H358 | | (SEQ ID NO: 459) | | |
| NCI- | hKRAS | TATCCAAACTGCCCTA | 14.3 | 10.8 |
| H358 | | (SEQ ID NO: 460) | | |

### Example 2: Inhibition of pERK

NCI-H358 tumor cell lines with KRAS mutation were plated at a density of 20,000 cells per well in a 96-well plates and were treated with both 5 nM and 20 nM of antisense oligonucleotide by transfection with Lipofectamine (Life Technology, USA). The transfection was conducted according to vendor's recommendation, with 0.3 μL Lipofectamine per well and incubated for 3 hours. After 4 days, cells were harvested and subjected to pERK AlphaLISA assay (Cat. ALSU-PERK-A10K, Perkin Elmer, USA). The pERK inhibition for each treatment was calculated by normalizing with a non-targeting control oligo and summarized in Table 6 below. Results show that oligonucleotides targeting KRAS inhibit pERK in NCI-H358 cells.

TABLE 6
pERK inhibition induced by KRAS knockdown
(antisense oligonucleotides constructed as
3-X-3 LNA gapmers, phosphorothioate-linked;
sequences from Tables 3 and 4)

| | Target | Antisense | % | |
|---|---|---|---|---|
| Cell | KRAS | Oligonucleotide | Inhibition | |
| lines | mRNA | (ASO) Sequence | 20 nM | 5 nM |
| NCI-H358 | Negative | CACGTCTATACACCAC | 0 | 0 |
| | control | (SEQ ID NO: 532) | | |
| NCI-H358 | Negative | TTTCTGAGGATTATCG | 5.1 | 11.3 |
| | control | (SEQ ID NO: 533) | | |
| NCI-H358 | hKRAS | TATCGTCAAGGCACTC | 49 | 34.5 |
| | | (SEQ ID NO: 458) | | |
| NCI-H358 | hKRAS | CCTCATTGCACTGTAC | 65 | 66.6 |
| | | (SEQ ID NO: 459) | | |
| NCI-H358 | hKRAS | TATCCAAACTGCCCTA | 41 | 30.7 |
| | | (SEQ ID NO: 460) | | |
| NCI-H358 | hKRAS G12C | TGCCTACGCCACAAGC | 59.1 | 49.4 |
| | | (SEQ ID NO: 461) | | |
| NCI-H358 | hKRAS G12C | GCCTACGCCACAAGCT | 59.6 | 45.3 |
| | | (SEQ ID NO: 462) | | |
| NCI-H358 | hKRAS G12C | CCTACGCCACAAGCTC | 49.6 | 49.1 |
| | | (SEQ ID NO: 463) | | |
| NCI-H358 | hKRAS G12C | CTACGCCACAAGCTCC | 55.5 | 47.7 |
| | | (SEQ ID NO: 464) | | |
| NCI-H358 | hKRAS G12C | TACGCCACAAGCTCCA | 72.4 | 69.8 |
| | | (SEQ ID NO: 465) | | |
| NCI-H358 | hKRAS G12C | ACGCCACAAGCTCCAA | 53.6 | 52.1 |
| | | (SEQ ID NO: 466) | | |
| NCI-H358 | hKRAS G12C | CGCCACAAGCTCCAAC | 43.1 | 53.6 |
| | | (SEQ ID NO: 467) | | |
| NCI-H358 | hKRAS G12C | GCCACAAGCTCCAACT | 36.9 | 44.1 |
| | | (SEQ ID NO: 468) | | |
| NCI-H358 | hKRAS G12C | CCACAAGCTCCAACTA | 48.3 | 40.7 |
| | | (SEQ ID NO: 469) | | |
| NCI-H358 | hKRAS G12C | CACAAGCTCCAACTAC | 47.3 | 36.4 |
| | | (SEQ ID NO: 470) | | |

### Example 3: Antisense Oligonucleotide Mediated Growth Inhibition of Various Cancer Cell Lines

Various tumor cell lines carrying different KRAS mutations were plated at a density of 800 cells per well in 384 well plates and were treated with both 5 uM and 1 uM of antisense oligonucleotide by coincubation. After 7 days, cell viability was measured by CellTiter-Glo® 2.0 assay (Promega, USA) according to vendor protocol, and calculated the growth inhibition against a non-targeting control oligo. The results are summarized in Tables 7-10. The results demonstrate that mutation-targeting antisense oligonucleotides are effective for targeting mutant KRAS.

TABLE 7
Antisense oligonucleotide mediated growth
inhibition in bronchioalveolar carcinoma
(antisense oligonucleotides constructed
as 3-X-3 LNA gapmers, phosphorothioate-
linked from Tables 3 and 4)

| Cell | Target | Antisense | % | |
|---|---|---|---|---|
| lines | KRAS | Oligonucleotide | Inhibition | |
| (G12C) | mRNA | (ASO) Sequence | 5 μM | 1 μM |
| NCI-H358 | Negative | CACGTCTATACACCAC | 0 | 0 |
| | control | (SEQ ID NO: 532) | | |
| NCI-H358 | hKRAS | GCTATTAGGAGTCTTT | 56.3 | 27.1 |
| | | (SEQ ID NO: 525) | | |
| NCI-H358 | hKRAS G12C | CTACGCCACAAGCTCC | 60.6 | 33 |
| | | (SEQ ID NO: 464) | | |
| NCI-H358 | hKRAS G12C | TACGCCACAAGCTCCA | 65.8 | 46.3 |
| | | (SEQ ID NO: 465) | | |
| NCI-H358 | hKRAS G12D | CCTACGCCATCAGCTC | −14.6 | −8.7 |
| | | (SEQ ID NO: 474) | | |
| NCI-H358 | hKRAS | CCTCATTGCACTGTAC | 56.9 | 32.7 |
| | | (SEQ ID NO: 459) | | |
| NCI-H358 | hKRAS G12C | ACGCCACAAGCTCCA | 63.5 | 38 |
| | | (SEQ ID NO: 481) | | |
| NCI-H358 | hKRAS G12C | CGCCACAAGCTCCA | 55.6 | 39.2 |
| | | (SEQ ID NO: 482) | | |
| NCI-H358 | hKRAS G12C | ACGCCACAAGCTCC | 55.7 | 37.8 |
| | | (SEQ ID NO: 483) | | |
| NCI-H358 | hKRAS G12D | CTACGCCATCAGCTC | −3.8 | −3.5 |
| | | (SEQ ID NO: 484) | | |
| NCI-H358 | hKRAS G12D | CTACGCCATCAGCT | −6.3 | 11.2 |
| | | (SEQ ID NO: 485) | | |
| NCI-H358 | hKRAS G12D | TACGCCATCAGCTC | −16.4 | −1.7 |
| | | (SEQ ID NO: 486) | | |
| NCI-H358 | hKRAS G12D | ACGCCATCAGCTCC | 2.6 | 1.4 |
| | | (SEQ ID NO: 487) | | |
| NCI-H358 | hKRAS G12A | CCTACGCCAGCAGCTC | 5.2 | 1.8 |
| | | (SEQ ID NO: 488) | | |
| NCI-H358 | hKRAS G12A | CTACGCCAGCAGCTC | 8.4 | 3.7 |
| | | (SEQ ID NO: 489) | | |
| NCI-H358 | hKRAS G12A | CTACGCCAGCAGCT | 1 | −7.9 |
| | | (SEQ ID NO: 490) | | |
| NCI-H358 | hKRAS G12A | TACGCCAGCAGCTC | 0.6 | −9.1 |
| | | (SEQ ID NO: 491) | | |
| NCI-H358 | hKRAS G12A | ACGCCAGCAGCTCC | 7.6 | 1.7 |
| | | (SEQ ID NO: 492) | | |
| NCI-H358 | hKRAS G12V | CCTACGCCAACAGCTC | 3.3 | −1.6 |
| | | (SEQ ID NO: 493) | | |
| NCI-H358 | hKRAS G12V | CTACGCCAACAGCTC | −6.2 | −2.5 |
| | | (SEQ ID NO: 494) | | |
| NCI-H358 | hKRAS G12V | CTACGCCAACAGCT | 10.2 | 7.6 |
| | | (SEQ ID NO: 495) | | |
| NCI-H358 | hKRAS G12V | TACGCCAACAGCTC | −15.4 | −15.9 |
| | | (SEQ ID NO: 496) | | |
| NCI-H358 | hKRAS G12V | ACGCCAACAGCTCC | 3.5 | 0.1 |
| | | (SEQ ID NO: 497) | | |

TABLE 8
Antisense oligonucleotide mediated growth
inhibition in lung large cell carcinoma
(antisense oligonucleotides constructed
as 3-X-3 LNA gapmers, phosphorothioate-
linked from Tables 3 and 4)

| Cell | Target | Antisense | % | |
|---|---|---|---|---|
| lines | KRAS | Oligonucleotide | Inhibition | |
| (G12V) | mRNA | (ASO) Sequence | 5 μM | 1 μM |
| LCLC-97TM1 | Negative | CACGTCTATACACCAC | 0 | 0 |
| | control | (SEQ ID NO: 532) | | |
| LCLC-97TM1 | hKRAS | GCTATTAGGAGTCTTT | 90.9 | 84.7 |
| | | (SEQ ID NO: 525) | | |
| LCLC-97TM1 | hKRAS G12C | CTACGCCACAAGCTCC | 21.8 | 7.9 |
| | | (SEQ ID NO: 464) | | |
| LCLC-97TM1 | hKRAS G12C | TACGCCACAAGCTCCA | 45.8 | 41.3 |
| | | (SEQ ID NO: 465) | | |
| LCLC-97TM1 | hKRAS G12D | CCTACGCCATCAGCTC | 57.4 | 17.3 |
| | | (SEQ ID NO: 474) | | |
| LCLC-97TM1 | hKRAS | CCTCATTGCACTGTAC | 88.3 | 77.2 |
| | | (SEQ ID NO: 459) | | |
| LCLC-97TM1 | hKRAS G12C | ACGCCACAAGCTCCA | 29.1 | 19.3 |
| | | (SEQ ID NO: 481) | | |
| LCLC-97TM1 | hKRAS G12C | CGCCACAAGCTCCA | 0.1 | 8.5 |
| | | (SEQ ID NO: 482) | | |
| LCLC-97TM1 | hKRAS G12C | ACGCCACAAGCTCC | 13.9 | 13.6 |
| | | (SEQ ID NO: 483) | | |
| LCLC-97TM1 | hKRAS G12D | CTACGCCATCAGCTC | 22.7 | 20.7 |
| | | (SEQ ID NO: 484) | | |
| LCLC-97TM1 | hKRAS G12D | CTACGCCATCAGCT | −21.5 | 13.4 |
| | | (SEQ ID NO: 485) | | |
| LCLC-97TM1 | hKRAS G12D | TACGCCATCAGCTC | 37.4 | 28.2 |
| | | (SEQ ID NO: 486) | | |
| LCLC-97TM1 | hKRAS G12D | ACGCCATCAGCTCC | 62.3 | 32.7 |
| | | (SEQ ID NO: 487) | | |
| LCLC-97TM1 | hKRAS G12A | CCTACGCCAGCAGCTC | 49.3 | 38.8 |
| | | (SEQ ID NO: 488) | | |
| LCLC-97TM1 | hKRAS G12A | CTACGCCAGCAGCTC | 61.7 | 34.3 |
| | | (SEQ ID NO: 489) | | |
| LCLC-97TM1 | hKRAS G12A | CTACGCCAGCAGCT | 22.3 | 18.3 |
| | | (SEQ ID NO: 490) | | |
| LCLC-97TM1 | hKRAS G12A | TACGCCAGCAGCTC | 50 | 22.7 |
| | | (SEQ ID NO: 491) | | |
| LCLC-97TM1 | hKRAS G12A | ACGCCAGCAGCTCC | 80.5 | 48.8 |
| | | (SEQ ID NO: 492) | | |
| LCLC-97TM1 | hKRAS G12V | CCTACGCCAACAGCTC | 78.7 | 66.9 |
| | | (SEQ ID NO: 493) | | |
| LCLC-97TM1 | hKRAS G12V | CTACGCCAACAGCTC | 84.1 | 70.1 |
| | | (SEQ ID NO: 494) | | |
| LCLC-97TM1 | hKRAS G12V | CTACGCCAACAGCT | 76.8 | 54.2 |
| | | (SEQ ID NO: 495) | | |
| LCLC-97TM1 | hKRAS G12V | TACGCCAACAGCTC | 87.2 | 65.1 |
| | | (SEQ ID NO: 496) | | |
| LCLC-97TM1 | hKRAS G12V | ACGCCAACAGCTCC | 85 | 71.3 |
| | | (SEQ ID NO: 497) | | |

TABLE 9
Antisense oligonucleotide mediated growth
inhibition in lung adenocarcinoma cells
(antisense oligonucleotides constructed
as 3-X-3 LNA gapmers, phosphorothioate-
linked from Tables 3 and 4)

| Cell | Target | Antisense | % | |
|---|---|---|---|---|
| lines | KRAS | Oligonucleotide | Inhibition | |
| (KRAS WT) | mRNA | (ASO) Sequence | 5 μM | 1 μM |
| NCI-H1975 | Negative | CACGTCTATACACCAC | 0 | 0 |
| | control | (SEQ ID NO: 532) | | |
| NCI-H1975 | hKRAS | GCTATTAGGAGTCTTT | 12.6 | 10.4 |
| | | (SEQ ID NO: 525) | | |
| NCI-H1975 | hKRAS G12C | CTACGCCACAAGCTCC | 29 | 12.4 |
| | | (SEQ ID NO: 464) | | |
| NCI-H1975 | hKRAS G12C | TACGCCACAAGCTCCA | 32.5 | 12.8 |
| | | (SEQ ID NO: 465) | | |
| NCI-H1975 | hKRAS G12D | CCTACGCCATCAGCTC | −4.8 | −2.4 |
| | | (SEQ ID NO: 474) | | |
| NCI-H1975 | hKRAS | CCTCATTGCACTGTAC | 9.6 | 9 |
| | | (SEQ ID NO: 459) | | |
| NCI-H1975 | hKRAS G12C | ACGCCACAAGCTCCA | 16.8 | 8 |
| | | (SEQ ID NO: 481) | | |
| NCI-H1975 | hKRAS G12C | CGCCACAAGCTCCA | 7.7 | 12 |
| | | (SEQ ID NO: 482) | | |
| NCI-H1975 | hKRAS G12C | ACGCCACAAGCTCC | 10.7 | 17 |
| | | (SEQ ID NO: 483) | | |
| NCI-H1975 | hKRAS G12D | CTACGCCATCAGCTC | −3.1 | 8.1 |
| | | (SEQ ID NO: 484) | | |
| NCI-H1975 | hKRAS G12D | CTACGCCATCAGCT | −6.1 | 5.9 |
| | | (SEQ ID NO: 485) | | |
| NCI-H1975 | hKRAS G12D | TACGCCATCAGCTC | −13.3 | 2.8 |
| | | (SEQ ID NO: 486) | | |
| NCI-H1975 | hKRAS G12D | ACGCCATCAGCTCC | −6.5 | 8.6 |
| | | (SEQ ID NO: 487) | | |
| NCI-H1975 | hKRAS G12A | CCTACGCCAGCAGCTC | 6.3 | 9.1 |
| | | (SEQ ID NO: 488) | | |
| NCI-H1975 | hKRAS G12A | CTACGCCAGCAGCTC | 2.7 | 14.1 |
| | | (SEQ ID NO: 489) | | |
| NCI-H1975 | hKRAS G12A | CTACGCCAGCAGCT | −1.9 | 8.5 |
| | | (SEQ ID NO: 490) | | |
| NCI-H1975 | hKRAS G12A | TACGCCAGCAGCTC | 3 | 13.5 |
| | | (SEQ ID NO: 491) | | |
| NCI-H1975 | hKRAS G12A | ACGCCAGCAGCTCC | 8.1 | 7.9 |
| | | (SEQ ID NO: 492) | | |
| NCI-H1975 | hKRAS G12V | CCTACGCCAACAGCTC | 8.9 | 11.8 |
| | | (SEQ ID NO: 493) | | |
| NCI-H1975 | hKRAS G12V | CTACGCCAACAGCTC | 6.9 | 8.4 |
| | | (SEQ ID NO: 494) | | |
| NCI-H1975 | hKRAS G12V | CTACGCCAACAGCT | −6.6 | 1.7 |
| | | (SEQ ID NO: 495) | | |
| NCI-H1975 | hKRAS G12V | TACGCCAACAGCTC | 10 | 8.8 |
| | | (SEQ ID NO: 496) | | |
| NCI-H1975 | hKRAS G12V | ACGCCAACAGCTCC | 17.8 | 7.1 |
| | | (SEQ ID NO: 497) | | |

TABLE 10
Antisense oligonucleotide mediated growth
inhibition in melanoma cells (antisense
oligonucleotides constructed as 3-X-3 LNA
gapmers, phosphorothioate-linked; from
Tables 3 and 4)

| Cell | | | | |
|---|---|---|---|---|
| lines | Target | Antisense | % | |
| (KRAS | KRAS | Oligonucleotide | Inhibition | |
| WT) | mRNA | (ASO) Sequence | 5 μM | 1 μM |
| A375 | Negative | CACGTCTATACACCAC | 0 | 0 |
| | control | (SEQ ID NO: 533) | | |
| A375 | hKRAS | GCTATTAGGAGTCTTT | −2.2 | −4.6 |
| | | (SEQ ID NO: 525) | | |
| A375 | hKRAS G12C | CTACGCCACAAGCTCC | 3.2 | −2.9 |
| | | (SEQ ID NO: 464) | | |
| A375 | hKRAS G12C | TACGCCACAAGCTCCA | −11.1 | −2.6 |
| | | (SEQ ID NO: 465) | | |
| A375 | hKRAS G12D | CCTACGCCATCAGCTC | −2.7 | −3.2 |
| | | (SEQ ID NO: 474) | | |
| A375 | hKRAS | CCTCATTGCACTGTAC | −5.3 | −13.6 |
| | | (SEQ ID NO: 459) | | |
| A375 | hKRAS G12C | ACGCCACAAGCTCCA | −4 | −10.4 |
| | | (SEQ ID NO: 481) | | |
| A375 | hKRAS G12C | CGCCACAAGCTCCA | 16.8 | 9.9 |
| | | (SEQ ID NO: 482) | | |
| A375 | hKRAS G12C | ACGCCACAAGCTCC | 12.2 | 6.2 |
| | | (SEQ ID NO: 483) | | |
| A375 | hKRAS G12D | CTACGCCATCAGCTC | 11.9 | 0 |
| | | (SEQ ID NO: 484) | | |
| A375 | hKRAS G12D | CTACGCCATCAGCT | 14.6 | 6.5 |
| | | (SEQ ID NO: 485) | | |
| A375 | hKRAS G12D | TACGCCATCAGCTC | 11.8 | 8 |
| | | (SEQ ID NO: 486) | | |
| A375 | hKRAS G12V | CCTACGCCAACAGCTC | 4.8 | −3.6 |
| | | (SEQ ID NO: 493) | | |
| A375 | hKRAS G12V | CTACGCCAACAGCTC | 2 | −1.5 |
| | | (SEQ ID NO: 494) | | |
| A375 | hKRAS G12V | CTACGCCAACAGCT | 2.3 | −4.7 |
| | | (SEQ ID NO: 495) | | |
| A375 | hKRAS G12V | TACGCCAACAGCTC | 4 | −3.8 |
| | | (SEQ ID NO: 496) | | |
| A375 | hKRAS G12V | ACGCCAACAGCTCC | 9.8 | 3.4 |
| | | (SEQ ID NO: 497) | | |

### Example 4: Mutated KRAS (G12C Mutation) Knockdown

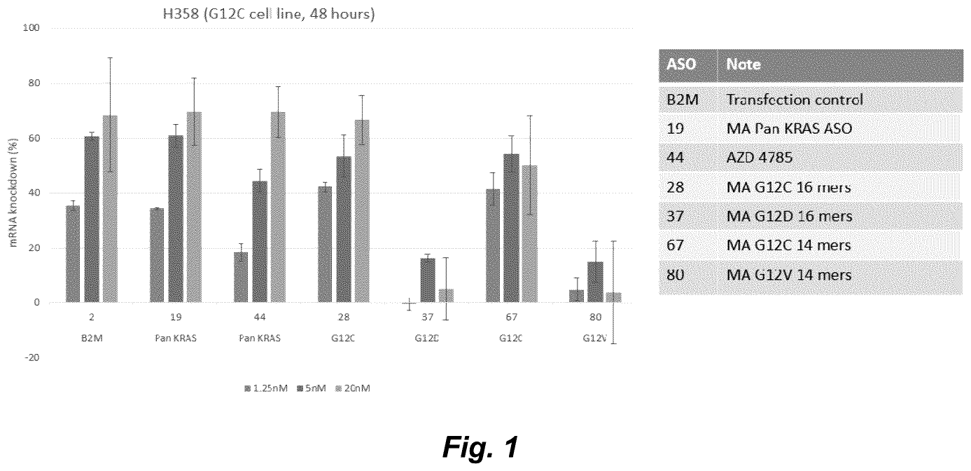

Cells were treated with Lipofectamine for transfection with an oligonucleotide described herein and harvested 48 hours post transfection for mRNA expression analysis. The data were normalized with PPIB mRNA levels individually and compared with non-targeting ASO to calculate percent knockdown. B2M denotes cell transfection with a transfection control. FIG. 1 illustrates mRNA knockdown of mutated KRAS having the G12C mutation (NCI-H358 cell) with ASO 28, ASO 37, ASO 67, or ASO 80 (Table 3). The knockdown of the mRNA of mutated KRAS having the G12C mutation was pronounced when mediated by ASO 28 and ASO 67.

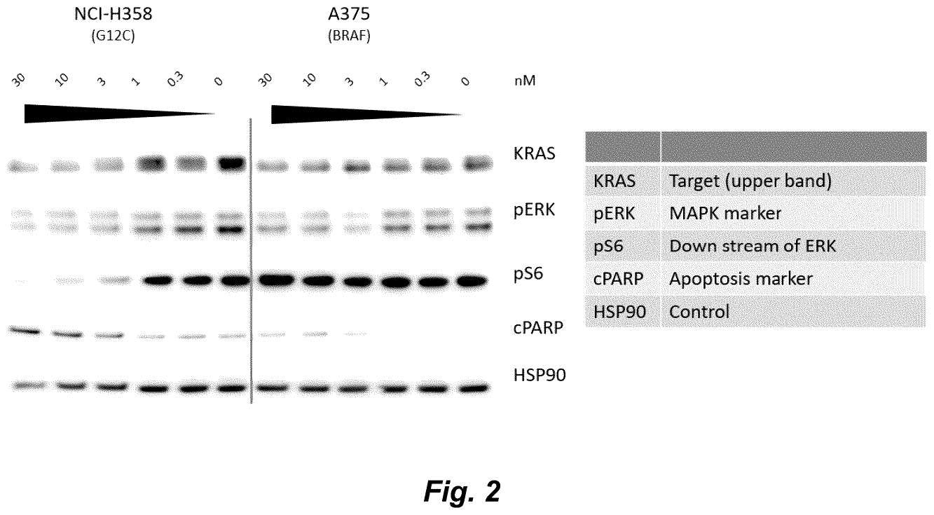

For protein level analysis, cells were seeded in 12 well plate at 10,000 cell/well and transfected with ASO/Lipofectamine (3 μl lipofectamine). Cells were harvested 3 days post transfection for protein expression analysis. The ASO used in the study was ASO 28 (G12C ASO, 16 mer), and the cell lines used were NCI-H358 (G12C, heterozygote for KRAS mutation) and A375 (KRAS wild type). FIG. 2 illustrates knockdown of protein expression of both KRAS and protein downstream of the KRAS-RAF-MEK-ERK signaling pathway (pERK, a MAPK marker; and pS6, downstream marker of ERK). FIG. 2 also illustrates increased apoptosis marker (cPARP) expression mediated by increased amount of the ASO (ASO 28) used for transfection.

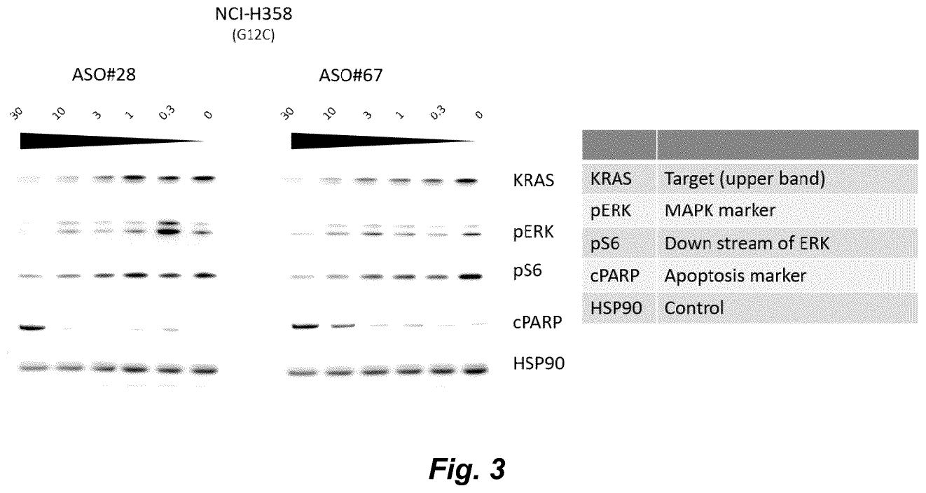

FIG. 3 illustrates another experiment of knockdown of protein expression of both KRAS and protein downstream of the KRAS-RAF-MEK-ERK signaling pathway (pERK, a MAPK marker; and pS6, downstream marker of ERK). Cells were seeded in 6-well plate at 100,000 cell/well and transfected with ASO/Lipofectamine (3 μl lipofectamine). Cells were harvested 3 days post transfection for protein expression analysis. The ASO used in the study was ASO 28 (G12C ASO, 16 mer) and ASO 67 (G12C ASO, 14 mer). The cell line used was NCI-H358 (G12C, heterozygote for KRAS mutation). Protein expression of both KRAS and protein downstream of the KRAS-RAF-MEK-ERK signaling pathway (pERK, a MAPK marker; and pS6, downstream marker of ERK) was decreased in cells transfected with ASO 28 or ASO 67 in a dose dependent manner. FIG. 3 also illustrates increased apoptosis marker (cPARP) expression mediated by increased amount of the ASO (ASO 28 and ASO 67) used for transfection.

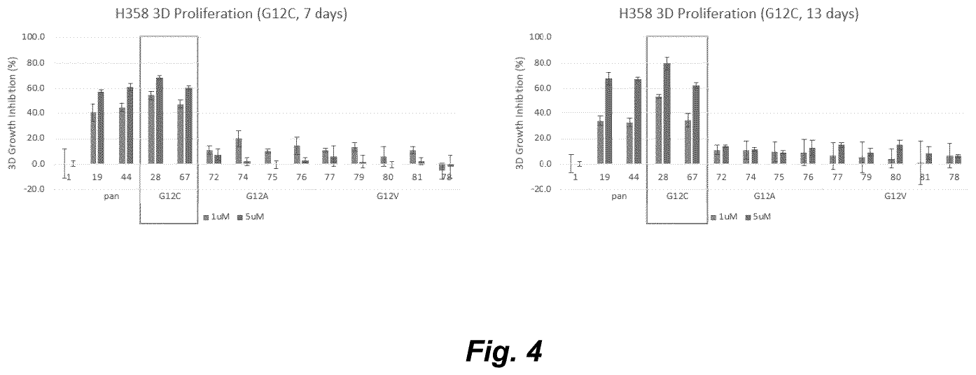

FIG. 4 illustrates 3D cell proliferation inhibition due to inhibiting expression of mutated KRAS by contacting the cell harboring the KRAS mutation (NCI-H358 cell having a KRAS G12C mutation) with the oligonucleotide described herein (e.g., ASO 28 or ASO 67). NCI-H358 cell (having G12C KRAS mutation) 3D growth was measured at 7 days (left) and at 13 days (right) after ASO treatment.

### Example 5: Mutated KRAS (G12V Mutation) Knockdown

Cells were seeded in 12 well plate at 10,000 cell/well and transfected with ASO/Lipofectamine (3 μl lipofectamine). Cells were harvested 3 days post transfection for protein expression analysis. The ASO used in the study was ASO 80 (G12V ASO, 14 mer), and the cell lines used were LCLC97TM1 (G12V, homozygote for KRAS mutation) and A375 (KRAS wild type).

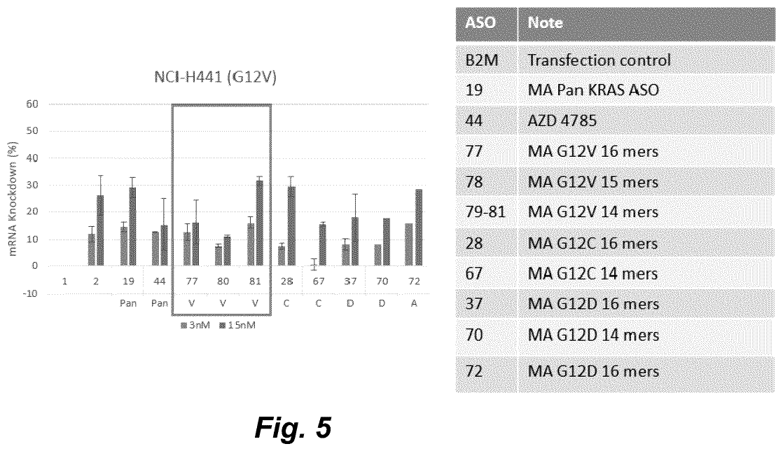

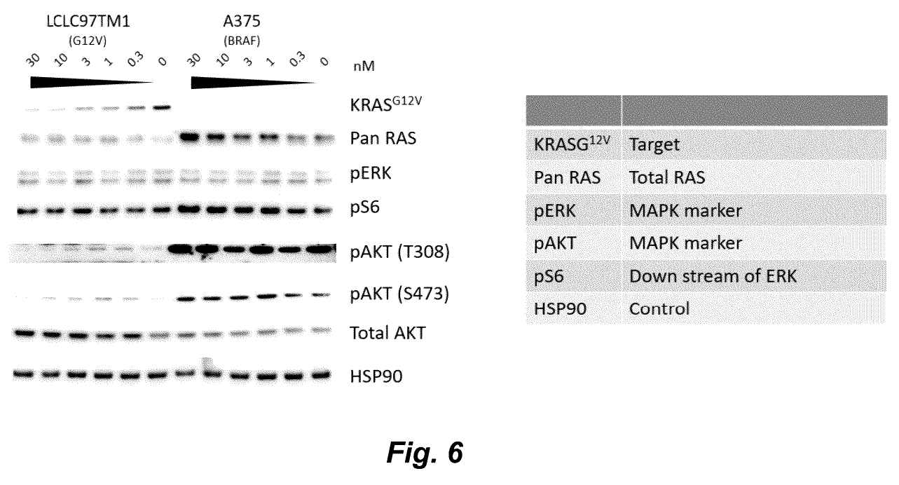

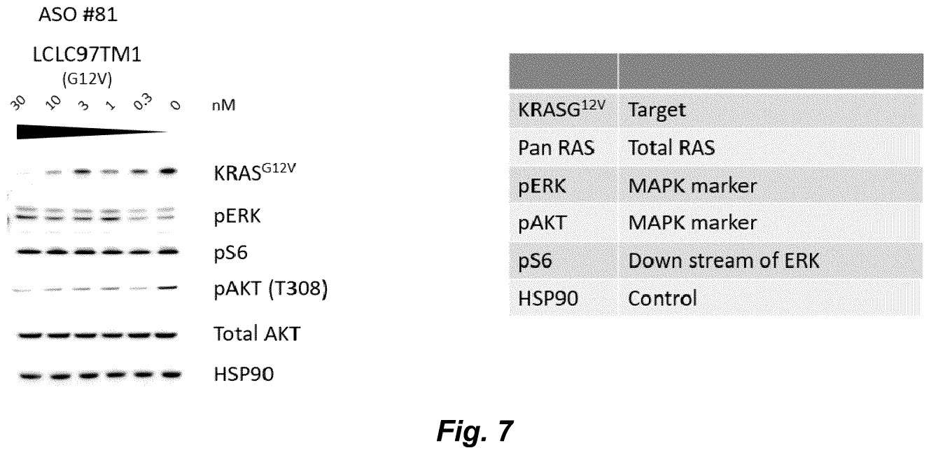

FIG. 5 illustrates mRNA knockdown of mutated KRAS having the G12V mutation (NCI-H441 cell) with ASO 77, ASO 80, or ASO 81. FIG. 6 illustrates knockdown (mediated by ASO 80) of protein expression of both G12V mutant KRAS and protein downstream of the KRAS-RAF-MEK-ERK signaling pathway (PERK and pAKT, both MAPK marker; and pS6, downstream marker of ERK). FIG. 7 illustrates knockdown (mediated by ASO 81) of protein expression of both G12V mutant KRAS and protein downstream of the KRAS-RAF-MEK-ERK signaling pathway (PERK and pAKT, both MAPK marker; and pS6, downstream marker of ERK). Cells were seeded in 12 well plate at 10,000 cell/well and transfected with ASO/Lipofectamine (3 μl lipofectamine). Cells were harvested 3 days post transfection for protein expression analysis. The ASO used in the study was ASO 80 (G12V ASO, 14 mer), and the cell lines used were LCLC97TM1 (G12V, homozygote for KRAS mutation) and A375 (KRAS wildtype).

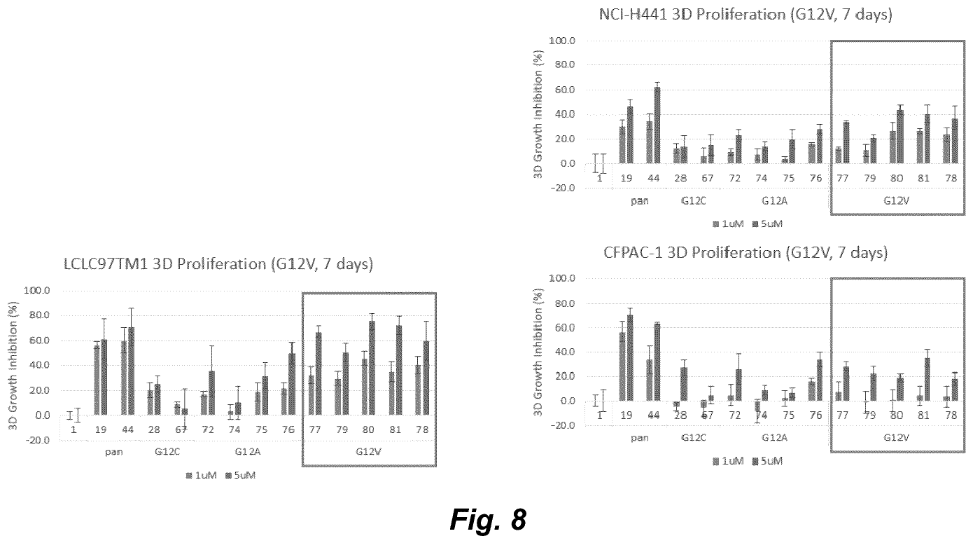

FIG. 8 illustrates 3D cell proliferation inhibition due to inhibiting expression of mutated KRAS by contacting the cell harboring the KRAS mutation (LCLC97TM1 cell, NCI-H441 cell, or CFPAC-1 cell) with oligonucleotides described herein (e.g., ASO 77-81). 3D growth was measured at 7 days after ASO treatment.

### Example 6: Mutated KRAS (G12A Mutation) Knockdown

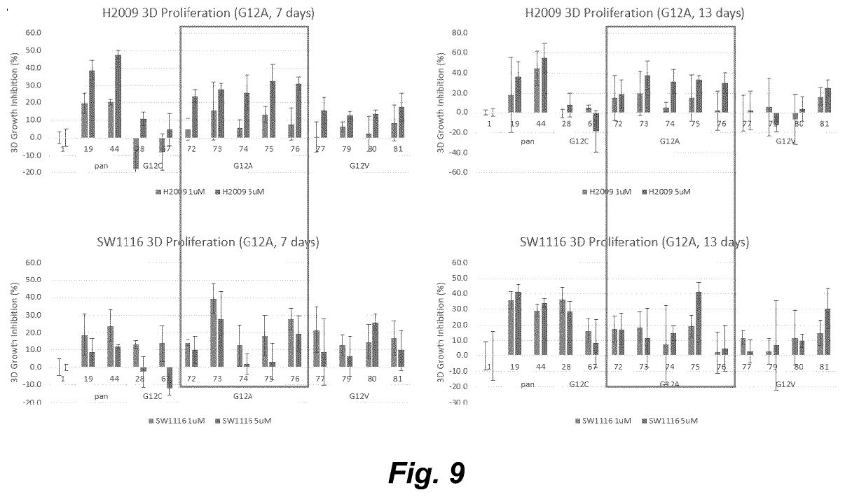

FIG. 9 illustrates 3D cell proliferation inhibition due to inhibiting expression of mutated KRAS by contacting the cell harboring the KRAS mutation (NCI-H2009 cell or SW1116 cell) with oligonucleotides described herein (e.g., see Table 3). 3D growth was measured at 7 days or 13 days after ASO treatment.

### Example 7: Mutated KRAS (G12D Mutation) Knockdown

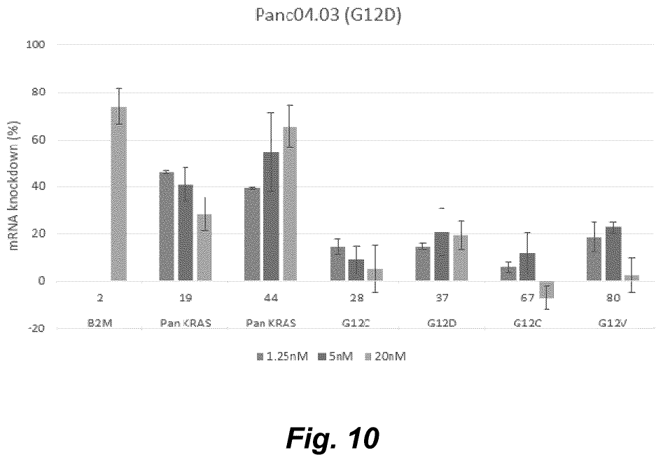

FIG. 10 illustrates mRNA knockdown of mutated KRAS having the G12D mutation (Panc04.03 cell) with ASO 37.

### Example 8: Binding Specificity of P19 Aptamer to PDAC Cells

### Cell Culture and Aptamer Folding Conditions

ATTC cells were grown in RPMI-1640 media supplemented with 10% fetal bovine serum, 100 U/ml penicillin and 100 U/ml streptomycin (Pen-Strep) and incubated at 37° C. in a humidified incubator containing 5% CO₂.

For aptamer folding conditions, the P19 labeled aptamer was resuspended to the desired concentration in RNAse free water. The resuspended aptamer was heated at 80° C. on a heat block for 5 minutes. The heat block is removed with P19 labeled aptamer tube and stored at room temperature until it reaches 25° C. The aptamer is stored on ice until use.

Assess P19 Aptamer Activity Conjugated with Fluorescent Dyes/Binding Assay

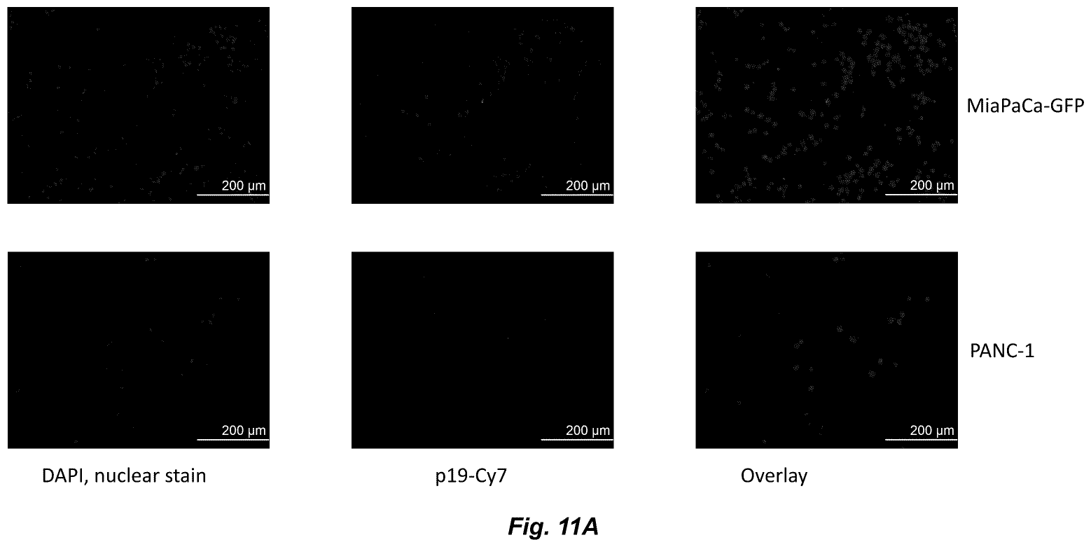

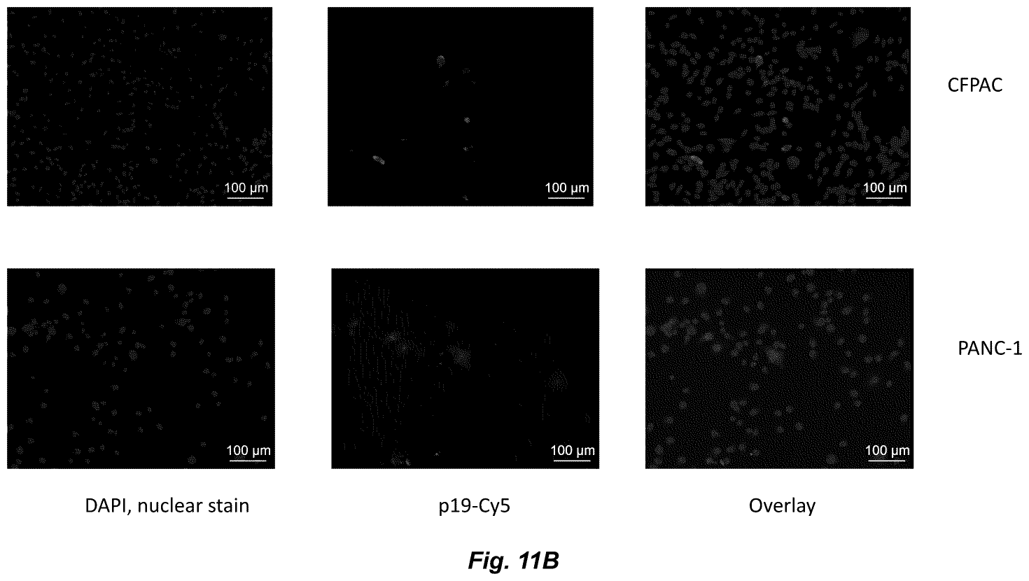

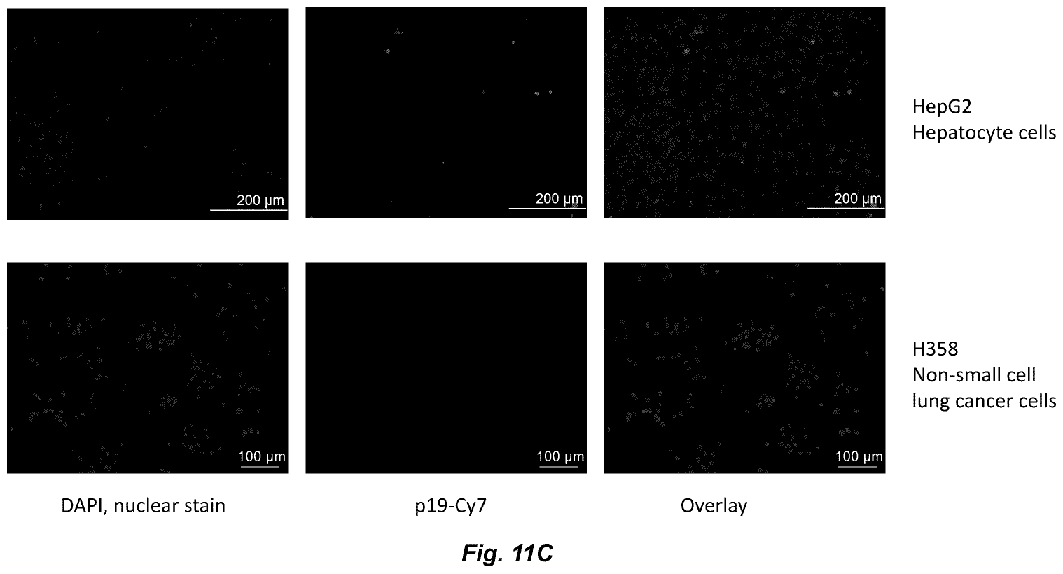

The P19 aptamer was conjugated to Cy7 or Cy5 and in-vitro binding was assessed in multiple cell lines. For binding assay, cells were plated at 70% confluency the previous day in 12-well plates. On the day of treatment, cells were washed twice with binding buffer (DPBS+5 mM MgCl) and incubated with 500 nM of folded aptamer for 30 minutes. After a 30-minute incubation, the media was removed and the cells were fixed with 4% formalin for 10 minutes at 37° C. to prepare for staining. The cells were washed twice with DPBS and stained with Hoescht 33342 at 1 μg/ml according to manufacturer's instructions (15 minutes at room temperature, store on ice in dark until visualization). Cells were imaged via Keyence BZX microscope using DAPI, Cy7 and Cy5 filters to detect nuclear staining and aptamer binding. The imaging results for binding specificity of the P19 aptamer is summarized in Table 11 and microscopy images are shown in FIGS. 11A-11C. The results show that P19 aptamer demonstrates specificity for PDAC cells (MIA PaCa-2-GFP[++], PANC-1 [+], CFPAC-1[+] in comparison to non-PDAC cells (hepatocytes[−], non-small cell lung cancer cells[−]).

TABLE 11
P19 Aptamer Demonstrates Specificity to PDAC
Cells in Comparison to non-PDAC Cell Lines

| Cell Line | PDAC/non-PDAC | Binding |
|---|---|---|
| MIA PaCa-2-GFP | PDAC | ++ |
| PANC-1 | PDAC | + |
| CFPAC-1 | PDAC | + |
| HepG2 | Non-PDAC, hepatocyte cells | − |
| NCI-H358 | Non-PDAC, non-small cell lung cancer cells | − |

### Example 9: Modulating Expression of KRAS

### Cell Culture Condition and In Vitro Transfection

ATCC cells were grown in RPMI-1640. The media were supplemented with 10% fetal bovine serum, 100 U/ml penicillin and 100 U/ml streptomycin (Pen-strep, or PS), and cells were incubated at 37° C. in a humidified incubator containing 5% CO₂. For antisense treatment, cells were plated at 70% confluency the previous day in 96-well plates. On the day of transfection, cells were washed once with OptiMEM medium and incubated in 90 μL OptiMEM. Transfection mixture was prepared in OptiMEM by mixing the antisense and the Lipofectamine RNAiMax transfection reagents at desired concentration, and 10 μL of the transfection mixture was added into each well, and incubated for 2 hours. At the end of the 2 hours, 10 μL of serum were added in to well, and made up the volume to 200 μL with respective culture medium for the cell line. Alternatively, media were replaced 2 hours post transfection or on the next day. The ASO treatment could also be done without transfection reagent. In this case, the ASOs were diluted with OptiMEM, and added into the cell culture in a volume less than 5% of the entire volume.

mRNA Knockdown Detection

Cells were harvested by lysis at 48 hours post transfection. The lysis and the follow up mRNA detection were conducted according to the Quantigene assay specified by the manufacturer (ThermoFisher). The ability of ASOs to knockdown desired mRNA was assessed as follows. MIA PaCa-2 (CRL-1420, ATCC) cells were plated in clear, flat bottom 96-well plate at 15,000 cells per well in RPMI1640 with 10% FBS for overnight. Cells were transfected with ASO at 30 nM, 10 nM, 3 nM and 1 nM complexed with RNAiMax according to manufacturer's instructions (Thermo Fisher). After 3 hours of transfection, transfection mixture was removed and replenished with RPMI1640+10% FBS and incubated for 48 hours. The mRNA quantitation was performed using QuantiGene from Thermo Fisher according to its instructions. The results for the KRAS mRNA knockdown by ASO are shown in Table 12.

### Inhibition of Cell Growth

The ability of ASOs to inhibit cell growth was assessed as follows. MIA PaCa-2 cells were plated in clear 384-well plates (S-Bio, #MS-9384UZ) at 600 cells per well in RPMI1640 with 10% FBS for overnight. Cells were treated with different concentration of ASO. After 7 days, cell viability was determined by measuring total ATP content using the Cell Titer Glo reagent (Promega, G7570) according to manufacturer's instructions. The results for cell proliferation inhibition are shown in Table 12.

P19 Aptamer Activity with Short Incubation Time

The ability of ASOs to inhibit PDAC cell growth was investigated using short term incubation time. It was shown previously that the P19 aptamer can bind to the surface of PDAC cells within 10 min of incubation. The P19 aptamer-conjugates described herein (e.g., ASO 840) using three different linkers (HEG spacer, C3 propyl spacer, and oligoA₁₂) were tested on MIA PaCa-2 (PDAC) using gymnosis with 6 days incubation or a shorter treatment (e.g., 30 min or 1 hour), removed ASO solution, and replenished with medium. ASO 840 is based on SEQ ID NO: 526, as a 3-8-3 LNA gapmer that is fully phosphorothioate linked. After 6 days, cell viability was determined by measuring total ATP content using the Cell Titer Glo reagent (Promega, G7570) according to manufacturer's instructions.

TABLE 12
Target mRNA knockdown and 3D cell proliferation
inhibition with ASO 840 and aptamer ASO 840.

| | | 3D Growth Inh (%) | mRNA KD (%) | |
|---|---|---|---|---|
| | | MIA PaCa-22 | MIA PaCa-2 | |
| | Target | 5 uM | 30 nM | |
| ASO (±linker) | Sequence | AVG | AVG | STDEV |
| ASO 840 without linker | G12C | 76.35 | 40.04 | 4.03758 |
| ASO 840 with a C3 propyl spacer | G12C | 74.25 | 54.42 | 1.209153 |
| ASO 840 with an oligoA₁₂ | G12C | 67.25 | 58.35 | 1.640488 |
| ASO 840 with a HEG spacer | G12C | 65.7 | 48.22 | 3.372899 |

Assess P19 Aptamer Activity Conjugated with ASO 840 and Different Linkers

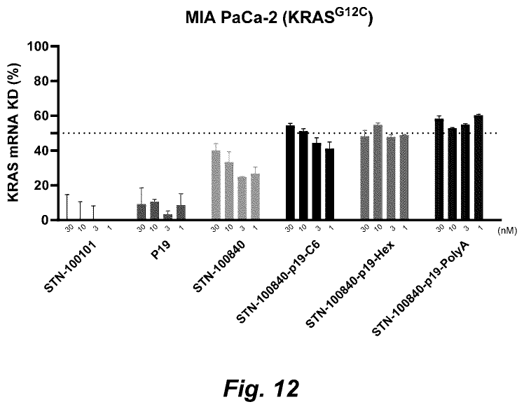

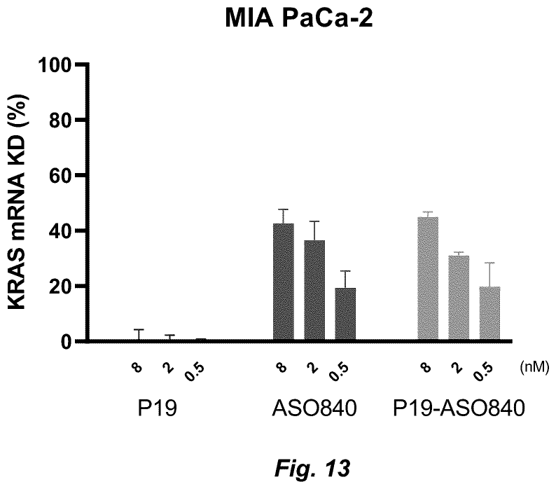

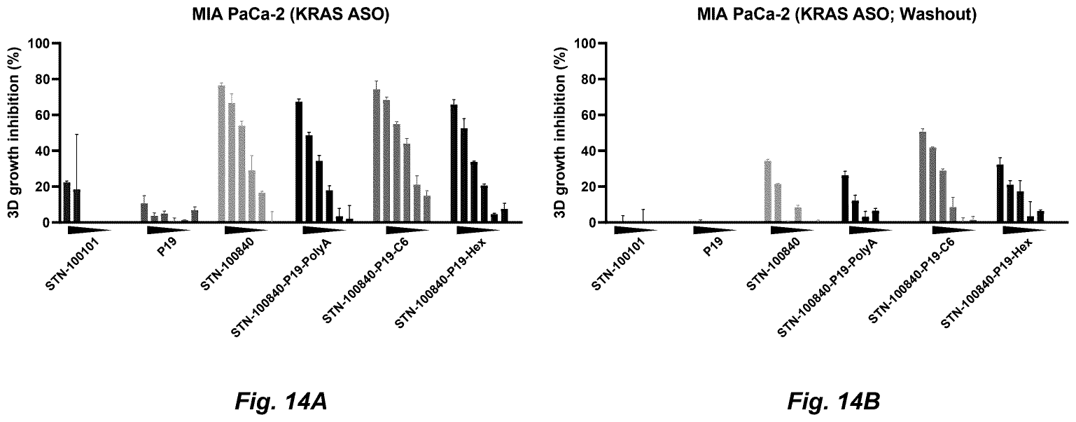

P19 aptamer was conjugated with ASO 840 using three different linkers (HEG spacer, C6 propyl spacer, and oligoA₁₂). Each of the conjugates were transfected at increasing nanomolar concentrations into MIA PaCa-2 (ATCC)] (KRAS G12C mutation) cells. The mRNA knockdown data were recorded as mRNA knockdown percentage. FIG. 12 shows P19 aptamer-conjugated ASO 840 preserves the specific, on target activity at all P19-conjugate concentrations tested using three different linkers (HEG spacer, C3 propyl spacer, and oligoA₁₂). Knockdown with ASO 840 is shown as a control. FIG. 13 further compares knockdown of KRAS mRNA in MIA PaCa-2 cells mediated by an antisense oligonucleotide (ASO 840) with and without linkage to P19 aptamer. FIG. 14A-14B illustrates 3D cell proliferation inhibition due to inhibiting expression of mutated KRAS by contacting the MIA PaCa-2 cells harboring the KRAS mutation (G12C mutation) with the P19 aptamer-conjugate (ASO 840) using three different linkers (HEG spacer, C3 propyl spacer, and oligoA₁₂). In FIG. 14A, MIA PaCa-2 cells were contacted P19 aptamer-conjugates for 6 days. In FIG. 14B, MIA PaCa-2 cells were contacted P19 aptamer-conjugates for 1 hour, then were washed out, and replenished with medium for 6 days. The washout experiment demonstrates that the P19 aptamer-conjugate (ASO 840) with a C6 propyl spacer showed increased 3D cell proliferation inhibition compared to the P19 aptamer alone, ASO 840 alone, and the other P19 aptamer-ASO 840 conjugates.

### Example 10: Modulating Expression of SOS

### Cell Culture Condition and In Vitro Transfection

ATCC cells were grown in RPMI-1640. The media were supplemented with 10% fetal bovine serum, 100 U/ml penicillin and 100 U/ml streptomycin (Pen-strep, or PS), and cells were incubated at 37° C. in a humidified incubator containing 5% CO₂. For antisense treatment, cells were plated at 70% confluency the previous day in 96-well plates. On the day of transfection, cells were washed once with OptiMEM medium and incubated in 90 μL OptiMEM. Transfection mixture was prepared in OptiMEM by mixing the antisense and the Lipofectamine RNAiMax transfection reagents at desired concentration, and 10 μL of the transfection mixture was added into each well, and incubated for 2 hours. At the end of the 2 hours, 10 μL of serum were added in to well, and made up the volume to 200 μL with respective culture medium for the cell line. Alternatively, media was replaced 2 hours post transfection or on the next day. The ASO treatment may also be done without a transfection reagent. In this instance, the ASOs were diluted with OptiMEM, then added into the cell culture in a volume less than 5% of the entire volume.

mRNA Knockdown Detection

Cells were harvested by lysis at 48 hours post transfection. The lysis and the follow up mRNA detection were conducted according to the Quantigen assay specified by the manufacturer (ThermoFisher).

The ability of ASOs to knockdown desired mRNA was assessed as follows. MIA PaCa-2 (CRL-1420, ATCC) cells were plated in clear, flat bottom 96-well plate at 15,000 cells per well in RPMI1640 with 10% FBS for overnight. Cells were transfected with ASO at 5 nM and 20 nM complexed with RNAiMax according to manufacturer's instructions (Thermo Fisher). After 3 hours of transfection, transfection mixture was removed and replenished with RPMI1640+10% FBS and incubated for 48 hours. The mRNA quantitation was performed using QuantiGene from Thermo Fisher according to instructions. The results for the SOS1 and SOS2 mRNA knockdown by ASO were shown in Table 13.

### Inhibition of Cell Growth

The ability of ASOs to inhibit cell growth was assessed as follows. MIA PaCa-2 cells were plated in clear 384-well plates (S-Bio, #MS-9384UZ) at 600 cells per well in RPMI1640 with 10% FBS for overnight. Cells were treated with different concentration of ASO. After 7 days, cell viability was determined by measuring total ATP content using the Cell Titer Glo reagent (Promega, G7570) according to manufacturer's instructions. The results for cell proliferation inhibition are shown in Table 13.

TABLE 13
SOS1 and SOS2 mRNA knockdown, and 3D cell proliferation inhibition
(ASOs constructed as 3-X-3, 2-X-3, 3-X-2, or 2-X-2 LNA gapmers,
fully phosphorothioate linked)

| | | | | | | | 3D |
|---|---|---|---|---|---|---|---|
| | | | | | | | Growth |
| | | | | | | | Inh (%) |
| | | | mRNA KD (%) | | | | MIA |
| | | | MIA PaCa-2 | | | | PaCa-2 |
| | | Target | SOS-120 nM | | SOS-2 20 nM | | 2.5 μM |
| SEQ ID NO: | Sequence | Sequence | AVG | STDEV | AVG | STDEV | AVG |
| SEQ ID NO: 534 | GCTCGAATGATCGGAA | SOS1 | 67.0 | 0.0 | | | |
| SEQ ID NO: 535 | ATTCGACTCACCACAG | SOS1 | 57.5 | 0.7 | | | |
| SEQ ID NO: 536 | TCGCCTATTGACTGCA | SOS1 | 73.0 | 5.7 | | | |
| SEQ ID NO: 537 | TCGCTCCTCTACATCT | SOS2 | | | 41.0 | 1.4 | |
| SEQ ID NO: 538 | TTCCCATCCATCGATA | SOS2 | | | 57.5 | 21.9 | |
| SEQ ID NO: 539 | CCCATCAAATGCACCA | SOS2 | | | 51.0 | 15.6 | |
| SEQ ID NO: 540 | AGGTCGTTGTTGGG | SOS1 | 46.6 | 12.7 | | | -10.9 |
| SEQ ID NO: 541 | GAGGTCGTTGTTGG | SOS1 | 68.9 | 0.5 | | | -3.0 |
| SEQ ID NO: 542 | CCGGAGGTCGTTGT | SOS1 | 67.3 | 6.2 | | | -13.0 |
| SEQ ID NO: 543 | GCGGAGGTCTCGCG | SOS1 | −9.2 | 13.6 | | | -15.1 |
| SEQ ID NO: 544 | AGTTGGGACTCCGA | SOS1 | 60.9 | 3.8 | | | 0.0 |
| SEQ ID NO: 545 | GCGTAGTTGGGACT | SOS1 | 60.6 | 6.0 | | | -0.3 |
| SEQ ID NO: 546 | CGCGTAGTTGGGAC | SOS1 | 86.7 | 2.0 | | | -15.5 |
| SEQ ID NO: 547 | CGGCATCCCGCACC | SOS1 | 17.9 | 6.0 | | | -13.0 |
| SEQ ID NO: 548 | GGAGAACGGACGCG | SOS1 | 84.8 | 2.3 | 0.0 | 0.0 | 1.5 |
| SEQ ID NO: 549 | TGGAGAACGGACGC | SOS1 | 80.2 | 2.4 | 26.2 | 5.0 | 0.4 |
| SEQ ID NO: 550 | TACCAGCCGTGGAG | SOS1 | 83.5 | 0.3 | | | -15.1 |
| SEQ ID NO: 551 | GTACCAGCCGTGGA | SOS1 | 83.9 | 0.4 | | | -4.2 |
| SEQ ID NO: 552 | CCCGACACAGGTAC | SOS1 | 11.0 | 34.2 | | | -5.1 |
| SEQ ID NO: 553 | GCCGGGCTAGCCCT | SOS1 | 68.9 | 3.5 | | | -8.7 |
| SEQ ID NO: 554 | AGCCGGGCTAGCCC | SOS1 | 67.1 | 3.1 | | | -14.1 |
| SEQ ID NO: 555 | AGCGCGGAACAGGG | SOS1 | 77.4 | 2.4 | 17.2 | 1.0 | -8.8 |
| SEQ ID NO: 556 | CAGCGCGGAACAGG | SOS1 | 80.7 | 2.3 | | | 3.7 |
| SEQ ID NO: 557 | CGCAGCGCGGAACA | SOS1 | 63.9 | 4.5 | | | -5.7 |
| SEQ ID NO: 558 | TCGCAGCGCGGAAC | SOS1 | 70.3 | 3.2 | | | 1.3 |
| SEQ ID NO: 559 | AGGGCGAGCTCGCA | SOS1 | 81.3 | 1.2 | 1.4 | 3.6 | -12.3 |
| SEQ ID NO: 560 | AAACTCGTAGGGCA | SOS1 | 68.9 | 0.1 | | | -7.4 |
| SEQ ID NO: 561 | AAAACTCGTAGGGC | SOS1 | 45.6 | 0.6 | | | -3.9 |
| SEQ ID NO: 562 | GGGCGCGTTCTCTT | SOS1 | 58.4 | 5.3 | | | -6.2 |
| SEQ ID NO: 563 | TTGGGCGCGTTCTC | SOS1 | 81.6 | 2.2 | | | -0.3 |
| SEQ ID NO: 564 | ACTTGGGCGCGTTC | SOS1 | 80.4 | 4.8 | 0.0 | 4.5 | -1.8 |
| SEQ ID NO: 565 | CGCCACTTGGGCGC | SOS1 | −5.3 | 18.3 | | | -4.3 |
| SEQ ID NO: 566 | GTAGTCCCCGCCAC | SOS1 | 18.9 | 3.6 | | | 0.8 |
| SEQ ID NO: 567 | TCGAGAGTAGGATG | SOS1 | 88.4 | 1.3 | 0.0 | 3.5 | -1.7 |
| SEQ ID NO: 568 | TTAGACTCGAGAGT | SOS1 | 68.8 | 0.4 | | | -3.8 |
| SEQ ID NO: 569 | TCATTAGACTCGAG | SOS1 | 81.7 | 1.2 | 18.0 | 1.0 | -0.2 |
| SEQ ID NO: 570 | ATCATTAGACTCGA | SOS1 | 89.9 | 4.5 | 0.0 | 0.0 | -2.9 |
| SEQ ID NO: 571 | GGCATAGCATATTT | SOS1 | 91.3 | 1.6 | | | 2.6 |
| SEQ ID NO: 572 | GAAGCACTTCGGGG | SOS1 | 55.5 | 2.9 | | | -8.8 |
| SEQ ID NO: 573 | ACGTTCCTCTACAT | SOS1 | 46.1 | 25.0 | | | 0.0 |
| SEQ ID NO: 574 | TATCAATTGGATGA | SOS1 | 66.5 | 0.5 | | | -10.1 |
| SEQ ID NO: 575 | CCCATTTATCAATT | SOS1 | 84.5 | 2.2 | 33.1 | 1.0 | -0.1 |
| SEQ ID NO: 576 | GCCCATTTATCAAT | SOS1 | 67.6 | 11.3 | 0.0 | 0.0 | -7.7 |
| SEQ ID NO: 577 | TGCCCATTTATCAA | SOS1 | 55.3 | 6.7 | | | -1.7 |
| SEQ ID NO: 578 | AGGACCTCCTTTAA | SOS1 | 46.1 | 10.1 | | | -8.8 |
| SEQ ID NO: 579 | TAGGACCTCCTTTA | SOS1 | 40.4 | 0.6 | | | -5.7 |
| SEQ ID NO: 580 | ATAACCTAGGACCT | SOS1 | 84.2 | 0.5 | 13.4 | 2.0 | 11.1 |
| SEQ ID NO: 581 | GCCGTATATTTCTT | SOS1 | 88.4 | 3.2 | 22.1 | 5.0 | 14.9 |
| SEQ ID NO: 582 | TGCCGTATATTTCT | SOS1 | 95.2 | 2.0 | 0.0 | 0.0 | 46.6 |
| (ASO 232) | | | | | | | |
| SEQ ID NO: 583 | TCATAATGCCGTAT | SOS1 | 91.0 | 1.8 | | | -2.8 |
| SEQ ID NO: 584 | ATTTCATAATGCCG | SOS1 | 92.8 | 0.3 | 80.9 | 3.0 | 40.9 |
| SEQ ID NO: 585 | GCTCTTCGTCAGTT | SOS1 | 70.4 | 3.8 | | | -5.6 |
| SEQ ID NO: 586 | GGAAGGCTCTTCGT | SOS1 | 87.6 | 0.2 | | | -4.2 |
| SEQ ID NO: 587 | TCATAGTAAGTTTG | SOS1 | 93.2 | 0.2 | -4.5 | 9.3 | -2.1 |
| SEQ ID NO: 588 | ATCATAGTAAGTTT | SOS1 | 53.0 | 6.7 | | | 0.1 |
| SEQ ID NO: 589 | TATATATTGTCGAA | SOS1 | 48.9 | 0.3 | | | 2.4 |
| SEQ ID NO: 590 | TAGATTTAGTTCCC | SOS1 | 89.0 | 1.0 | -0.7 | 5.8 | -9.6 |
| SEQ ID NO: 591 | GCGACTAAATATAT | SOS1 | 86.4 | 1.1 | -9.4 | 2.4 | -6.5 |
| SEQ ID NO: 592 | TATCTACTATGCGA | SOS1 | 86.1 | 3.9 | -3.8 | 7.8 | 2.2 |
| SEQ ID NO: 593 | ATGGCCCAGTAACT | SOS1 | 60.1 | 0.5 | | | -0.6 |
| SEQ ID NO: 594 | CTTCTATATGGCCC | SOS1 | 36.4 | 1.0 | | | -3.0 |
| SEQ ID NO: 595 | CCTACTAGTGGATG | SOS1 | 53.4 | 13.1 | | | 0.7 |
| SEQ ID NO: 596 | CGATTCATATGGAT | SOS1 | 88.2 | 2.4 | -3.4 | 0.8 | -8.7 |
| SEQ ID NO: 597 | ACGATTCATATGGA | SOS1 | 88.1 | 2.0 | 11.3 | 2.4 | -0.4 |
| SEQ ID NO: 598 | CATACGATTCATAT | SOS1 | 67.6 | 4.3 | | | -7.8 |
| SEQ ID NO: 599 | TCGAGCATACGATT | SOS1 | 90.6 | 1.7 | 12.9 | 5.0 | -8.7 |
| SEQ ID NO: 600 | ATCTCGAGCATACG | SOS1 | 68.4 | 0.3 | | | 6.9 |
| SEQ ID NO: 601 | ATATCTCGAGCATA | SOS1 | 30.9 | 1.6 | | | 3.8 |
| SEQ ID NO: 602 | CGCAAAATATCTCG | SOS1 | 81.9 | 2.8 | 15.4 | 7.9 | 6.5 |
| SEQ ID NO: 603 | AACGATCATGAAAA | SOS1 | 31.8 | 6.4 | | | 5.9 |
| SEQ ID NO: 604 | ACTAAGGAAACGAT | SOS1 | 62.3 | 1.1 | | | 4.4 |
| SEQ ID NO: 605 | GCCTATTGACTGCA | SOS1 | 50.3 | 3.7 | | | 1.3 |
| SEQ ID NO: 606 | CGCCTATTGACTGC | SOS1 | 87.6 | 1.4 | 23.1 | 2.0 | 3.2 |
| SEQ ID NO: 607 | CCTTCGCCTATTGA | SOS1 | 71.7 | 0.6 | | | 3.1 |
| SEQ ID NO: 608 | ACCTTCGCCTATTG | SOS1 | 76.9 | 3.0 | | | 7.6 |
| SEQ ID NO: 609 | GAAACCTTCGCCTA | SOS1 | 89.5 | 1.2 | 25.4 | 2.0 | 11.8 |
| SEQ ID NO: 610 | GCCTGGGTAAAACA | SOS1 | 68.1 | 0.9 | | | -12.7 |
| SEQ ID NO: 611 | TTCGTTTTGCAAGA | SOS1 | 83.6 | 1.6 | 24.8 | 3.0 | 37.9 |
| SEQ ID NO: 612 | CTTCGTTTTGCAAG | SOS1 | 20.2 | 2.2 | | | -6.4 |
| SEQ ID NO: 613 | AACCGACATGCAGA | SOS1 | 75.9 | 1.8 | 11.8 | 9.7 | 6.6 |
| SEQ ID NO: 614 | ACTATAAAACCGAC | SOS1 | 79.1 | 1.9 | 11.5 | 4.4 | 7.0 |
| SEQ ID NO: 615 | GACTATAAAACCGA | SOS1 | 83.5 | 0.5 | 12.6 | 7.0 | -1.1 |
| SEQ ID NO: 616 | CTAGTTGTTTCCCC | SOS1 | 77.0 | 2.5 | 5.2 | 5.0 | 3.9 |
| SEQ ID NO: 617 | ACAACACTGTCCAA | SOS1 | 64.1 | 1.8 | 69.9 | 0.0 | 28.3 |
| SEQ ID NO: 618 | ATTACAACACTGTC | SOS1 | 78.2 | 3.4 | 77.8 | 1.0 | 26.2 |
| SEQ ID NO: 619 | CATTACAACACTGT | SOS1 | 77.3 | 6.2 | 67.7 | 4.0 | 3.6 |
| SEQ ID NO: 620 | TCATTACAACACTG | SOS1 | 92.4 | 0.2 | 90.0 | 0.0 | 35.8 |
| SEQ ID NO: 621 | AATTCATTACAACA | SOS1 | 43.9 | 4.2 | 36.0 | 1.0 | -6.2 |
| SEQ ID NO: 622 | TACACGTGTAAGAG | SOS1 | 91.7 | 0.7 | | | -13.9 |
| SEQ ID NO: 623 | CTACACGTGTAAGA | SOS1 | 76.7 | 0.3 | | | -16.5 |
| SEQ ID NO: 624 | TAAGACGATATTCT | SOS1 | 75.4 | 2.4 | | | 6.1 |
| SEQ ID NO: 625 | TTTAAGACGATATT | SOS1 | 31.1 | 2.0 | | | 1.2 |
| SEQ ID NO: 626 | TCGCATAAAAAACT | SOS1 | 22.0 | 14.5 | | | 3.9 |
| SEQ ID NO: 627 | CCTTTCGCATAAAA | SOS1 | 66.3 | 4.3 | | | -6.1 |
| SEQ ID NO: 628 | TACCTTTCGCATAA | SOS1 | 72.2 | 3.4 | | | -0.5 |
| SEQ ID NO: 629 | TGTACCTTTCGCAT | SOS1 | 64.2 | 2.6 | | | 2.7 |
| SEQ ID NO: 630 | TACTCCGGTACTGT | SOS1 | 76.5 | 2.6 | | | 5.5 |
| SEQ ID NO: 631 | ACGTAAGCCTCTCT | SOS1 | 69.2 | 5.1 | | | -2.7 |
| SEQ ID NO: 632 | TGGTACGTAAGCCT | SOS1 | 29.4 | 6.4 | | | 0.2 |
| SEQ ID NO: 633 | TATGGTACGTAAGC | SOS1 | 76.9 | 2.4 | | | 11.2 |
| SEQ ID NO: 634 | ACATATGGTACGTA | SOS1 | 59.2 | 6.3 | | | -4.7 |
| SEQ ID NO: 635 | GTACATATGGTACG | SOS1 | 42.8 | 3.0 | | | -3.2 |
| SEQ ID NO: 636 | TGCGTACATATGGT | SOS1 | 72.3 | 8.4 | | | 2.5 |
| SEQ ID NO: 637 | CGAACAAAATTGGG | SOS1 | 80.6 | 2.1 | 80.6 | 2.0 | 32.8 |
| SEQ ID NO: 638 | CCGAACAAAATTGG | SOS1 | 12.3 | 10.4 | | | -6.9 |
| SEQ ID NO: 639 | GAAATGTCCGAACA | SOS1 | 83.9 | 2.1 | | | -7.5 |
| SEQ ID NO: 640 | TAAGAAATGTCCGA | SOS1 | 72.0 | 0.2 | | | 1.2 |
| SEQ ID NO: 641 | ATTATAAGACTCAG | SOS1 | 75.7 | 0.4 | | | -1.7 |
| SEQ ID NO: 642 | CTATGCGATCAGCT | SOS1 | 77.2 | 4.1 | 0.0 | 0.0 | 0.8 |
| SEQ ID NO: 643 | CTATAGCTATGCGA | SOS1 | 87.0 | 0.6 | 65.6 | 10.0 | 2.2 |
| SEQ ID NO: 644 | TGCACTCAAGGGTT | SOS1 | 69.7 | 1.1 | 49.7 | 7.0 | 4.1 |
| SEQ ID NO: 645 | TCGCAGTTGCACAG | SOS1 | 80.8 | 4.1 | | | -8.5 |
| SEQ ID NO: 646 | TTAATACTCGCAGT | SOS1 | 75.2 | 3.1 | | | -1.6 |
| SEQ ID NO: 647 | TTTAATACTCGCAG | SOS1 | 80.6 | 1.4 | | | 8.3 |
| SEQ ID NO: 648 | GTGCCGACATACAT | SOS1 | 78.1 | 3.5 | | | -0.3 |
| SEQ ID NO: 649 | CAGTGCCGACATAC | SOS1 | 73.3 | 0.4 | 8.3 | 2.0 | 0.0 |
| SEQ ID NO: 650 | TTCCATTCGTTGCA | SOS1 | 56.7 | 1.5 | | | 0.5 |
| SEQ ID NO: 651 | CTTCCATTCGTTGC | SOS1 | 41.8 | 4.1 | | | 5.3 |
| SEQ ID NO: 652 | GATGGATTCAACCC | SOS1 | 72.8 | 5.1 | | | 0.3 |
| SEQ ID NO: 653 | ATGCCACTCAACTG | SOS1 | 73.8 | 2.3 | 47.3 | 5.0 | 20.4 |
| SEQ ID NO: 654 | GGTCTGCTTATATG | SOS1 | 64.3 | 5.6 | | | -11.6 |
| SEQ ID NO: 655 | TCGAGCAATTTCTA | SOS1 | 81.9 | 4.2 | 69.9 | 4.0 | 23.7 |
| SEQ ID NO: 656 | AAGTGAGTTGTCGA | SOS1 | 72.5 | 3.1 | 59.5 | 5.0 | 15.9 |
| SEQ ID NO: 657 | TCGGTATAGATCTG | SOS1 | 90.6 | 0.2 | 62.1 | 5.0 | -0.1 |
| SEQ ID NO: 658 | TACAGCTCGGTATA | SOS1 | 63.0 | 1.7 | | | -3.0 |
| SEQ ID NO: 659 | CTGTACAGCTCGGT | SOS1 | 80.5 | 1.0 | -15.0 | 9.0 | -3.0 |
| SEQ ID NO: 660 | TCCAACTAATTCTG | SOS1 | 79.2 | 2.5 | | | 2.1 |
| SEQ ID NO: 661 | AGATTAGGAGAGTT | SOS1 | 79.6 | 2.7 | | | 5.2 |
| SEQ ID NO: 662 | ATGTCGAATCATTT | SOS1 | 28.8 | 19.0 | | | -7.3 |
| SEQ ID NO: 663 | GGTGGTATGTCGAA | SOS1 | 60.2 | 8.3 | | | -6.1 |
| SEQ ID NO: 664 | TGGTGGTATGTCGA | SOS1 | 68.3 | 3.2 | 51.0 | 21.0 | 14.6 |
| SEQ ID NO: 665 | CTCAATAATTCGAC | SOS1 | 50.6 | 0.8 | | | -3.9 |
| SEQ ID NO: 666 | TCTCAATAATTCGA | SOS1 | 5.9 | 17.3 | | | -5.2 |
| SEQ ID NO: 667 | TGGTCTAGTCTGTA | SOS1 | 32.1 | 3.0 | 78.2 | 4.0 | 5.3 |
| SEQ ID NO: 668 | GGCGACTTGGTATT | SOS1 | 88.0 | 0.4 | 47.5 | 4.0 | -5.6 |
| SEQ ID NO: 669 | TCTGGCGACTTGGT | SOS1 | 85.5 | 2.5 | 77.8 | 0.0 | 0.9 |
| SEQ ID NO: 670 | ATTAATAGACCTGA | SOS1 | 72.5 | 0.3 | | | -17.7 |
| SEQ ID NO: 671 | GATTAATAGACCTG | SOS1 | 76.5 | 0.2 | | | -5.0 |
| SEQ ID NO: 672 | TGGATTAATAGACC | SOS1 | 57.9 | 0.9 | | | -8.8 |
| SEQ ID NO: 673 | CGTAAACAGTAAGG | SOS1 | 61.3 | 2.2 | | | -2.1 |
| SEQ ID NO: 674 | ACTCGTAAACAGTA | SOS1 | 15.5 | 15.5 | | | -6.3 |
| SEQ ID NO: 675 | GATTCTACTCGTAA | SOS1 | 39.1 | 2.8 | | | -7.0 |
| SEQ ID NO: 676 | CTGATTCTACTCGT | SOS1 | 61.7 | 2.8 | | | 0.3 |
| SEQ ID NO: 677 | CATCGGATTCAAGT | SOS1 | 63.7 | 5.0 | | | 2.5 |
| SEQ ID NO: 678 | TCCCATCGGATTCA | SOS1 | 26.0 | 5.0 | | | 0.4 |
| SEQ ID NO: 679 | CTATTTCCCATCGG | SOS1 | 78.3 | 3.4 | | | -1.2 |
| SEQ ID NO: 680 | TCGTGGTTCTATTT | SOS1 | 76.9 | 2.8 | | | -2.8 |
| SEQ ID NO: 681 | GGTTTCGTGGTTCT | SOS1 | 63.6 | 4.5 | 75.1 | 1.0 | 45.3 |
| SEQ ID NO: 682 | TAGGGTTTCGTGGT | SOS1 | 73.0 | 3.5 | 62.8 | 1.0 | 26.7 |
| SEQ ID NO: 683 | CTTAGGGTTTCGTG | SOS1 | 64.9 | 3.0 | | | 2.1 |
| SEQ ID NO: 684 | AGAGGCTTAGGGTT | SOS1 | 52.5 | 8.8 | 18.4 | 5.0 | 28.6 |
| SEQ ID NO: 685 | GGGGATAGCTATAT | SOS1 | 38.2 | 1.2 | | | 20.7 |
| SEQ ID NO: 686 | ATGGTACCTGGTCT | SOS1 | 57.0 | 6.2 | 34.8 | 8.0 | 16.6 |
| SEQ ID NO: 687 | TCCTACTATAACTA | SOS1 | 53.3 | 5.9 | | | 1.4 |
| SEQ ID NO: 688 | GGATCCTACTATAA | SOS1 | 66.7 | 1.9 | | | -6.5 |
| SEQ ID NO: 689 | GTTAACGGTGTTCT | SOS1 | 81.4 | 1.8 | | | -2.6 |
| SEQ ID NO: 690 | TGTTAACGGTGTTC | SOS1 | 78.6 | 0.2 | | | 1.8 |
| SEQ ID NO: 691 | GGCGGAGGTGTTAA | SOS1 | 63.9 | 7.4 | | | 0.5 |
| SEQ ID NO: 692 | TGATCGGAATCAAA | SOS1 | 30.1 | 1.9 | | | -11.0 |
| SEQ ID NO: 693 | AATGATCGGAATCA | SOS1 | 39.6 | 1.9 | | | -21.8 |
| SEQ ID NO: 694 | TCGAATGATCGGAA | SOS1 | 63.7 | 1.7 | | | -14.2 |
| SEQ ID NO: 695 | GCTCGAATGATCGG | SOS1 | 86.0 | 0.8 | -1.1 | 2.0 | -11.7 |
| SEQ ID NO: 696 | AAAGGGCTCGAATG | SOS1 | 51.1 | 0.5 | | | -2.0 |
| SEQ ID NO: 697 | GAAAAGGGCTCGAA | SOS1 | 44.4 | 5.2 | | | 3.3 |
| SEQ ID NO: 698 | TTAAACTTATAGAT | SOS1 | 2.9 | 14.3 | | | -4.8 |
| SEQ ID NO: 699 | CAGATTCTGGTCGT | SOS1 | 51.7 | 1.4 | | | -12.9 |
| SEQ ID NO: 700 | AATGCTTAGACATA | SOS1 | 41.4 | 2.9 | | | -2.6 |
| SEQ ID NO: 701 | GTGGGTTGCCTAGG | SOS1 | 74.0 | 0.1 | 37.2 | 5.0 | 10.5 |
| SEQ ID NO: 702 | TCGTGGTGAATAGG | SOS1 | 85.6 | 1.5 | 66.6 | 4.0 | 11.9 |
| SEQ ID NO: 703 | TATCGTGGTGAATA | SOS1 | 69.3 | 2.5 | | | -0.5 |
| SEQ ID NO: 704 | ATATTGAATATCGT | SOS1 | 69.6 | 0.0 | | | -3.5 |
| SEQ ID NO: 705 | CGGTCTGATATTGA | SOS1 | 80.1 | 2.4 | 69.2 | 3.0 | 18.1 |
| SEQ ID NO: 706 | GGTCCGGTCTGATA | SOS1 | 73.5 | 1.4 | 65.3 | 10.0 | -4.8 |
| SEQ ID NO: 707 | AGGTCCGGTCTGAT | SOS1 | 63.0 | 2.2 | -1.8 | 7.0 | 8.1 |
| SEQ ID NO: 708 | ATAGAGGTCCGGTC | SOS1 | 68.7 | 5.3 | | | -0.9 |
| SEQ ID NO: 709 | GATAGAGGTCCGGT | SOS1 | 72.2 | 1.0 | 37.6 | 6.0 | 2.9 |
| SEQ ID NO: 710 | TCGTGGTGGTAATA | SOS1 | 63.3 | 8.8 | | | 3.5 |
| SEQ ID NO: 711 | CACAGGTTCTCGTG | SOS1 | 19.8 | 7.5 | | | -13.6 |
| SEQ ID NO: 712 | AAGTGCTTTGTCGT | SOS1 | 74.4 | 2.0 | 23.3 | 0.0 | 37.1 |
| SEQ ID NO: 713 | AGTCGTTAGTGTTT | SOS1 | 87.3 | 2.1 | 11.9 | 10.0 | 6.5 |
| SEQ ID NO: 714 | GAAGAGTCGTTAGT | SOS1 | 64.8 | 2.2 | | | -0.7 |
| SEQ ID NO: 715 | AGAAGAGTCGTTAG | SOS1 | 78.9 | 0.2 | | | -1.7 |
| SEQ ID NO: 716 | AATTACATCTAGGT | SOS1 | 75.3 | 3.8 | | | -3.7 |
| SEQ ID NO: 717 | GTCGGTCTTTCCAT | SOS1 | 63.0 | 2.5 | 60.4 | 2.0 | 13.8 |
| SEQ ID NO: 718 | TAGGTCGGTCTTTC | SOS1 | 60.2 | 2.4 | | | -1.9 |
| SEQ ID NO: 719 | ACACTAGACTGTGT | SOS1 | 27.5 | 13.7 | | | -2.8 |
| SEQ ID NO: 720 | TGTCATGTGGGCCT | SOS1 | 53.4 | 0.3 | 22.9 | 6.0 | 20.1 |
| SEQ ID NO: 721 | AGTACAGTGCCGGG | SOS1 | 62.8 | 9.1 | 53.3 | 0.0 | 12.8 |
| SEQ ID NO: 722 | CCGTAATAATCTGT | SOS1 | 75.8 | 6.1 | | | 7.5 |
| SEQ ID NO: 723 | CCCATTCAGTTCAC | SOS1 | 74.6 | 1.5 | | | -4.9 |
| SEQ ID NO: 724 | CGGTACCCATTCAG | SOS1 | 74.0 | 2.6 | | | 0.4 |
| SEQ ID NO: 725 | ATAGATGAACAGTC | SOS1 | 84.4 | 2.1 | | | 0.5 |
| SEQ ID NO: 726 | ACAATCTAGCATAG | SOS1 | 86.0 | 2.2 | | | 4.9 |
| SEQ ID NO: 727 | TGGACTACCAGTAA | SOS1 | 81.4 | 2.4 | 48.3 | 1.0 | 10.4 |
| SEQ ID NO: 728 | TACGAGCCATGACA | SOS1 | 85.7 | 0.9 | | | 0.9 |
| SEQ ID NO: 729 | GATAGCTACGAGCC | SOS1 | 91.6 | 2.2 | | | 0.4 |
| SEQ ID NO: 730 | GGGATAGCTACGAG | SOS1 | 84.9 | 1.7 | 66.6 | 7.0 | 12.1 |
| SEQ ID NO: 731 | GAGGGATAGCTACG | SOS1 | 83.2 | 2.4 | 5.0 | 31.0 | -0.4 |
| SEQ ID NO: 732 | CATGAGCGGTGTCA | SOS1 | 7.3 | 19.1 | | | -10.5 |
| SEQ ID NO: 733 | TCAACTATGTGTAA | SOS1 | 38.4 | 7.6 | | | -14.3 |
| SEQ ID NO: 734 | ACGTGCTTTTATGT | SOS1 | 38.5 | 4.6 | | | -16.0 |
| SEQ ID NO: 735 | CACAAAACGTGCTT | SOS1 | 49.5 | 11.0 | | | -18.7 |
| SEQ ID NO: 736 | GTACCACAAAACGT | SOS1 | 61.3 | 11.1 | | | -3.6 |
| SEQ ID NO: 737 | GCGTACCACAAAAC | SOS1 | 28.4 | 15.2 | | | 7.2 |
| SEQ ID NO: 738 | CAAGGCGTACCACA | SOS1 | 84.5 | 0.6 | 0.0 | 0.0 | 20.8 |
| SEQ ID NO: 739 | AAACCAAGGCGTAC | SOS1 | 69.6 | 14.5 | | | -15.0 |
| SEQ ID NO: 740 | ATACCCATCCTATT | SOS1 | 41.2 | 0.2 | | | -7.8 |
| SEQ ID NO: 741 | GTTGCTTAGGAACT | SOS1 | 63.4 | 5.2 | | | -10.0 |
| SEQ ID NO: 742 | GGTAGATGTTCTAT | SOS1 | 26.9 | 58.2 | | | -6.6 |
| SEQ ID NO: 743 | CATACGCTTTATGA | SOS1 | −0.1 | 3.9 | | | -7.9 |
| SEQ ID NO: 744 | TACATACGCTTTAT | SOS1 | 74.2 | 5.3 | | | -9.9 |
| SEQ ID NO: 745 | ACAGTACATACGCT | SOS1 | 81.4 | 2.5 | 0.0 | 0.0 | 1.2 |
| SEQ ID NO: 746 | AACAGTACATACGC | SOS1 | 74.9 | 4.5 | 0.0 | 0.0 | 5.2 |
| SEQ ID NO: 747 | ATCGTGATGTACAT | SOS1 | 69.0 | 14.3 | 0.0 | 0.0 | -7.1 |
| SEQ ID NO: 748 | ACTGTACTATCGTG | SOS1 | 12.1 | 19.0 | | | -2.7 |
| SEQ ID NO: 749 | ATAGGATAGACTGC | SOS1 | 10.6 | 32.1 | | | 3.1 |
| SEQ ID NO: 750 | GGACGCTTTTCAAA | SOS1 | 67.3 | 3.3 | | | 2.9 |
| SEQ ID NO: 751 | ATGTTTTAGGACGC | SOS1 | 88.8 | 0.7 | 0.0 | 0.0 | 9.3 |
| SEQ ID NO: 752 | CTAGTAAGTTGGCT | SOS1 | 82.8 | 0.1 | 0.0 | 0.0 | -6.8 |
| SEQ ID NO: 753 | GCGGCTCTAGTAAG | SOS1 | 67.8 | 3.5 | | | -2.9 |
| SEQ ID NO: 754 | TGTAGCGGCTCTAG | SOS1 | 72.9 | 2.9 | | | 21.2 |
| SEQ ID NO: 755 | TAGTTGTAGCGGCT | SOS1 | 84.1 | 10.3 | 0.0 | 0.0 | 1.0 |
| SEQ ID NO: 756 | ATTAGTTGTAGCGG | SOS1 | 93.6 | 0.8 | | | -8.0 |
| SEQ ID NO: 757 | TCTATCATGATTAG | SOS1 | 45.1 | 2.1 | | | -12.8 |
| SEQ ID NO: 758 | AGCCGAAGACATAC | SOS1 | 35.8 | 9.6 | | | -11.0 |
| SEQ ID NO: 759 | TTAGCCGAAGACAT | SOS1 | 29.8 | 4.3 | | | -11.8 |
| SEQ ID NO: 760 | CATTAGCCGAAGAC | SOS1 | 37.2 | 4.3 | 0.0 | 0.0 | -2.8 |
| SEQ ID NO: 761 | AACACATTAGCCGA | SOS1 | 1.9 | 33.0 | | | 12.9 |
| SEQ ID NO: 762 | TATTGAAGGCTAAC | SOS1 | 10.9 | 9.1 | | | -9.2 |
| SEQ ID NO: 763 | GACTAGATAGGTTG | SOS1 | 68.2 | 7.3 | | | -7.8 |
| SEQ ID NO: 764 | AGACTAGATAGGTT | SOS1 | 69.6 | 2.4 | | | -4.4 |
| SEQ ID NO: 765 | ATAGACTAGATAGG | SOS1 | 81.8 | 2.1 | | | -3.2 |
| SEQ ID NO: 766 | TTATAGACTAGATA | SOS1 | 9.6 | 33.4 | | | -14.2 |
| SEQ ID NO: 767 | TGGTCTCCTAGAAC | SOS1 | 15.3 | 11.5 | | | -5.7 |
| SEQ ID NO: 768 | TAAGGGCATCCAAT | SOS1 | 46.9 | 3.9 | | | -7.4 |
| SEQ ID NO: 769 | TGTCAACTAGTTAA | SOS1 | 50.6 | 1.8 | | | -6.8 |
| SEQ ID NO: 770 | TGTAATGTCCAGGA | SOS1 | 66.0 | 2.9 | | | 0.3 |
| SEQ ID NO: 771 | ACCCCCTTGTAATG | SOS1 | 32.4 | 18.6 | | | -4.7 |
| SEQ ID NO: 772 | CATTTACCCCCTTG | SOS1 | 41.2 | 0.9 | | | 5.2 |
| SEQ ID NO: 773 | GCGAACTCTCAATG | SOS1 | −2.1 | 11.5 | | | -5.8 |
| SEQ ID NO: 774 | ATTAAGCGAACTCT | SOS1 | 80.6 | 0.7 | 0.0 | 0.0 | 9.0 |
| SEQ ID NO: 775 | AGTAACCTACTAAA | SOS1 | 51.7 | 1.0 | | | -18.2 |
| SEQ ID NO: 776 | AGAATAGTAACCTA | SOS1 | 57.6 | 14.2 | | | -7.3 |
| SEQ ID NO: 777 | TTAGTGTGGCATGC | SOS1 | 87.5 | 3.5 | | | 5.2 |
| SEQ ID NO: 778 | CAGCTGACATAGTA | SOS1 | 76.1 | 7.8 | | | -18.1 |
| SEQ ID NO: 779 | TCATAATATAGCCT | SOS1 | 50.1 | 0.5 | | | -0.7 |
| SEQ ID NO: 780 | GGTTTACATAAGGT | SOS1 | 76.2 | 5.1 | 0.0 | 0.0 | 9.1 |
| SEQ ID NO: 781 | CGTAGTCACACAAT | SOS1 | 66.3 | 8.2 | 0.0 | 0.0 | 2.7 |
| SEQ ID NO: 782 | TTGCGTAGTCACAC | SOS1 | 55.1 | 0.4 | | | -0.5 |
| SEQ ID NO: 783 | CCTATACTTGCGTA | SOS1 | 39.2 | 10.5 | | | -3.3 |
| SEQ ID NO: 784 | ATGTATAAGGGATT | SOS1 | 5.7 | 21.7 | | | 1.3 |
| SEQ ID NO: 785 | TTAAGTAGAGCTCA | SOS1 | 33.8 | 4.4 | | | 0.8 |
| SEQ ID NO: 786 | AGTCGACAGAAAAT | SOS1 | 77.9 | 7.9 | | | -4.9 |
| SEQ ID NO: 787 | GCTCACAGTCGACA | SOS1 | 78.3 | 4.3 | | | -20.3 |
| SEQ ID NO: 788 | TGCTCACAGTCGAC | SOS1 | 81.7 | 6.1 | 0.0 | 0.0 | -2.5 |
| SEQ ID NO: 789 | ACTTAATGCACTAT | SOS1 | 61.9 | 0.6 | 0.0 | 0.0 | -7.4 |
| SEQ ID NO: 790 | TACTTAATGCACTA | SOS1 | 60.6 | 13.9 | | | -16.0 |
| SEQ ID NO: 791 | GACCCACTGTTAGC | SOS1 | 20.6 | 13.2 | | | -7.8 |
| SEQ ID NO: 792 | TGACCCACTGTTAG | SOS1 | 60.7 | 10.3 | | | 1.9 |
| SEQ ID NO: 793 | TGTTATGCATCTTG | SOS1 | 75.5 | 2.2 | 0.0 | 0.0 | 5.8 |
| SEQ ID NO: 794 | CCTAAGCCACACCA | SOS1 | 30.1 | 9.5 | | | 2.1 |
| SEQ ID NO: 795 | CACGTCTCAACTAA | SOS1 | 28.6 | 13.6 | | | -13.2 |
| SEQ ID NO: 796 | AGCAAATAACCGTG | SOS1 | 59.9 | 10.7 | | | -9.1 |
| SEQ ID NO: 797 | TAGCAAATAACCGT | SOS1 | 26.7 | 8.1 | | | -3.9 |
| SEQ ID NO: 798 | CCGACCGACAGAGC | SOS1 | 77.6 | 9.4 | | | 5.9 |
| SEQ ID NO: 799 | ATCAAAGACCGACC | SOS1 | 81.2 | 2.8 | | | -16.8 |
| SEQ ID NO: 800 | ACATCAAAGACCGA | SOS1 | 73.8 | 10.4 | | | -6.3 |
| SEQ ID NO: 801 | GTTAGGTACAAAGG | SOS1 | 71.7 | 3.4 | | | 3.7 |
| SEQ ID NO: 802 | ACATGGTTAGGTAC | SOS1 | 68.0 | 2.8 | | | -12.2 |
| SEQ ID NO: 803 | AGCCGTTATTTTCA | SOS1 | 63.7 | 6.1 | | | -2.0 |
| SEQ ID NO: 804 | CAGCCGTTATTTTC | SOS1 | 73.7 | 4.3 | | | -2.0 |
| SEQ ID NO: 805 | AAAATAACAGCCGT | SOS1 | 62.5 | 1.3 | | | 4.8 |
| SEQ ID NO: 806 | AGTTTATCTTAGTA | SOS1 | 15.8 | 12.3 | | | -8.5 |
| SEQ ID NO: 807 | GGCTATGTAAGGCA | SOS1 | 0.0 | 0.0 | | | -7.7 |
| SEQ ID NO: 808 | AATTTCATAATGCC | SOS1 | 87.8 | 0.9 | 63.0 | 2.6 | 14.7 |
| SEQ ID NO: 809 | TTACAACACTGTCC | SOS1 | 74.3 | 0.4 | 74.8 | 1.0 | 19.2 |
| SEQ ID NO: 810 | ATTCATTACAACAC | SOS1 | 12.4 | 2.0 | 16.5 | 8.9 | 1.7 |
| SEQ ID NO: 811 | AAATTGGGATCTGC | SOS1 | 78.2 | 0.7 | 74.9 | 3.0 | 8.5 |
| SEQ ID NO: 812 | ATATGCCACTCAAC | SOS1 | 76.2 | 3.7 | 29.1 | 6.3 | 13.6 |
| SEQ ID NO: 813 | GCAATTTCTATTGG | SOS1 | 73.5 | 2.3 | 66.3 | 0.5 | 7.2 |
| SEQ ID NO: 814 | GAGCAATTTCTATT | SOS1 | 68.9 | 8.3 | 23.1 | 7.4 | 0.3 |
| SEQ ID NO: 815 | GCTGGTTCTATTTC | SOS1 | 57.2 | 2.9 | 35.3 | 0.0 | 0.1 |
| SEQ ID NO: 816 | GTTTCGTGGTTCTA | SOS1 | 67.0 | 7.9 | 20.4 | 1.8 | 25.8 |
| SEQ ID NO: 817 | TGCACTTGTTCCTG | SOS2 | | | 23.4 | 6.8 | -18.2 |
| SEQ ID NO: 818 | GAGTGGGATGCACT | SOS2 | | | 25.8 | 12.8 | -9.2 |
| SEQ ID NO: 819 | TAGCAGATTGTGCA | SOS2 | | | 8.3 | 3.5 | -13.0 |
| SEQ ID NO: 820 | TCGTTTTTCTATAG | SOS2 | | | 39.9 | 4.3 | -3.0 |
| SEQ ID NO: 821 | CGTTTTCGTTTTTC | SOS2 | | | 27.7 | 9.6 | -8.3 |
| SEQ ID NO: 822 | CTTCGTTTTCGTTT | SOS2 | 29.9 | 4.9 | 35.2 | 18.7 | 14.6 |
| SEQ ID NO: 823 | TTCTTCGTTTTCGT | SOS2 | | | 18.5 | 1.0 | -12.1 |
| SEQ ID NO: 824 | ATGGTAGTCCACTT | SOS2 | | | -3.9 | 15.8 | -6.9 |
| SEQ ID NO: 825 | TACATGGTAGTCCA | SOS2 | 23.4 | 10.6 | 37.3 | 5.0 | 19.0 |
| SEQ ID NO: 826 | GATACATGGTAGTC | SOS2 | | | 29.2 | 1.2 | -7.4 |
| SEQ ID NO: 827 | GACACTTTAATGTC | SOS2 | | | 0.0 | 0.0 | -3.9 |
| SEQ ID NO: 828 | GAGAGAAACCAAAC | SOS2 | | | 17.5 | 2.7 | -0.1 |
| SEQ ID NO: 829 | ATAGTAGTTTAATT | SOS2 | | | -21.1 | 0.4 | -3.9 |
| SEQ ID NO: 830 | GCGATTTCAGTTCT | SOS2 | | | 38.1 | 0.2 | -9.5 |
| SEQ ID NO: 831 | ATATTTAATTCCCG | SOS2 | 29.3 | 6.2 | 47.0 | 11.9 | 8.6 |
| SEQ ID NO: 832 | ACACTTTTATGATC | SOS2 | | | 26.7 | 16.8 | -4.7 |
| SEQ ID NO: 833 | TCGAAACACTTTTA | SOS2 | | | 32.2 | 0.5 | 6.4 |
| SEQ ID NO: 834 | TTCTCGAAACACTT | SOS2 | | | 35.4 | 3.1 | -9.8 |
| SEQ ID NO: 835 | TTCATGTATATCTG | SOS2 | 81.1 | 2.8 | 86.3 | 0.8 | 71.4 |
| (ASO 674) | | | | | | | |
| SEQ ID NO: 836 | GTCAATTCATGTAT | SOS2 | | | 29.9 | 1.8 | -8.0 |
| SEQ ID NO: 837 | CTTCAATCAAACCT | SOS2 | | | 11.0 | 12.2 | -2.0 |
| SEQ ID NO: 838 | GTGATAATGTTTCA | SOS2 | | | 37.8 | 5.4 | -0.1 |
| SEQ ID NO: 839 | GTGTAGAGCAACTG | SOS2 | | | 12.4 | 2.8 | -5.3 |
| SEQ ID NO: 840 | AAGTGTAGAGCAAC | SOS2 | | | 8.6 | 4.6 | -1.0 |
| SEQ ID NO: 841 | CTGAAAGTGTAGAG | SOS2 | | | 28.9 | 6.4 | -1.3 |
| SEQ ID NO: 842 | AGCATAAGACGTGG | SOS2 | 30.7 | 5.5 | 57.3 | 4.0 | -3.1 |
| SEQ ID NO: 843 | ACAGTGATACACTG | SOS2 | | | 0.0 | 0.0 | -5.1 |
| SEQ ID NO: 844 | GTAACTCAAAGTAG | SOS2 | | | 7.5 | 8.4 | 4.8 |
| SEQ ID NO: 845 | TTGTTCTTCACTAC | SOS2 | | | -3.6 | 6.0 | 1.3 |
| SEQ ID NO: 846 | AATAGCTTGGTTCA | SOS2 | 28.3 | 8.4 | 36.4 | 2.9 | 21.1 |
| SEQ ID NO: 847 | AGTAATAGCTTGGT | SOS2 | | | -5.2 | 7.9 | -0.2 |
| SEQ ID NO: 848 | CTTGTAAATTCGGT | SOS2 | | | 21.2 | 3.1 | -4.6 |
| SEQ ID NO: 849 | CGACGTCTAGGTGA | SOS2 | | | 5.2 | 5.3 | -1.2 |
| SEQ ID NO: 850 | TAATTGGTGACTAT | SOS2 | | | 5.9 | 5.4 | -1.0 |
| SEQ ID NO: 851 | CTTAATTGGTGACT | SOS2 | | | 22.4 | 1.2 | 1.2 |
| SEQ ID NO: 852 | CCCATCCATCGATA | SOS2 | | | 6.4 | 1.2 | -7.3 |
| SEQ ID NO: 853 | AATTCATTACAACA | SOS2 | | | -14.3 | 17.7 | -6.0 |
| SEQ ID NO: 854 | CGATTCTTGTCAAT | SOS2 | | | 22.7 | 8.0 | -4.0 |
| SEQ ID NO: 855 | CACCGATTCTTGTC | SOS2 | | | 4.7 | 22.0 | -3.3 |
| SEQ ID NO: 856 | TTGGCACCGATTCT | SOS2 | | | 33.1 | 5.7 | 0.0 |
| SEQ ID NO: 857 | ATGTTTGGCACCGA | SOS2 | −5.4 | 21.2 | 50.6 | 3.4 | 26.5 |
| SEQ ID NO: 858 | GCCGTTCATGTTTG | SOS2 | | | 42.8 | 3.1 | -2.5 |
| SEQ ID NO: 859 | TGATTAGGTTTACA | SOS2 | | | 3.9 | 2.5 | 4.1 |
| SEQ ID NO: 860 | CACTACTGTAACCT | SOS2 | | | 20.5 | 2.3 | 2.1 |
| SEQ ID NO: 861 | CATGACAAATTTTT | SOS2 | | | -8.2 | 4.4 | -7.7 |
| SEQ ID NO: 862 | GCTCACAAGTATCT | SOS2 | 19.5 | 0.2 | 46.8 | 0.4 | -6.3 |
| SEQ ID NO: 863 | ATGCATGCTTGTGC | SOS2 | | | 38.9 | 2.9 | 0.2 |
| SEQ ID NO: 864 | TCAAATGCATGCTT | SOS2 | 18.3 | 3.7 | 47.8 | 5.9 | -3.3 |
| SEQ ID NO: 865 | GCAGCAAATATTAT | SOS2 | | | 18.7 | 13.6 | 0.0 |
| SEQ ID NO: 866 | TTCGATCTAGAGTA | SOS2 | | | 42.6 | 2.8 | -2.6 |
| SEQ ID NO: 867 | AACATTCGATCTAG | SOS2 | | | -16.4 | 3.7 | -2.7 |
| SEQ ID NO: 868 | GGTAATCTCAGTGG | SOS2 | | | 25.3 | 1.1 | -6.7 |
| SEQ ID NO: 869 | CAAAACGATATACT | SOS2 | | | -2.9 | 2.1 | 0.0 |
| SEQ ID NO: 870 | CTACAAAACGATAT | SOS2 | | | 1.5 | 8.2 | -8.8 |
| SEQ ID NO: 871 | ACAATGTTTTCCTC | SOS2 | 31.6 | 4.7 | 34.2 | 11.3 | 39.2 |
| SEQ ID NO: 872 | TGCAAGTTGTCTTC | SOS2 | | | 24.6 | 4.0 | -7.3 |
| SEQ ID NO: 873 | CCTTTAATAATGGG | SOS2 | | | 17.1 | 12.9 | -6.2 |
| SEQ ID NO: 874 | TGTTAACCTTTCAA | SOS2 | | | -1.0 | 9.9 | -8.7 |
| SEQ ID NO: 875 | ATTGGGATCTGCAT | SOS2 | | | 5.7 | 17.3 | 6.5 |
| SEQ ID NO: 876 | ACGATATGTGGTAA | SOS2 | | | 33.8 | 7.8 | 4.6 |
| SEQ ID NO: 877 | GCTCAGCAATTCCT | SOS2 | | | -11.7 | 7.0 | -4.2 |
| SEQ ID NO: 876 | GAATTTCAAACCGT | SOS2 | | | 34.1 | 0.7 | 5.9 |
| SEQ ID NO: 877 | CGTCAGTAGGTTCT | SOS2 | | | 31.9 | 7.1 | -0.3 |
| SEQ ID NO: 878 | AGGTCTGCACTGAT | SOS2 | | | 32.7 | 4.3 | 15.7 |
| SEQ ID NO: 879 | TCTTTTAAGGTCTG | SOS2 | −8.4 | 5.1 | 42.9 | 5.9 | 12.8 |
| SEQ ID NO: 880 | AATCTTTTAAGGTC | SOS2 | | | 17.7 | 0.4 | 7.7 |
| SEQ ID NO: 881 | GCGAAATCTTTTAA | SOS2 | | | 2.8 | 1.1 | 3.8 |
| SEQ ID NO: 882 | CCTAAGTTGTACTG | SOS2 | | | 18.8 | 3.5 | 2.0 |
| SEQ ID NO: 883 | GATCCTAAGTTGTA | SOS2 | | | 27.3 | 4.2 | 8.4 |
| SEQ ID NO: 884 | CCCTCTTACACTTG | SOS2 | | | 28.8 | 4.1 | -5.7 |
| SEQ ID NO: 885 | GACTCTACCCATTT | SOS2 | | | 26.5 | 0.1 | 14.4 |
| SEQ ID NO: 886 | AGCAATTGACTCTA | SOS2 | | | 39.8 | 2.1 | -16.4 |
| SEQ ID NO: 887 | TTAGCAATTGACTC | SOS2 | | | 40.1 | 2.3 | -12.0 |
| SEQ ID NO: 888 | ATCTTAGCAATTGA | SOS2 | | | 24.7 | 7.7 | -8.2 |
| SEQ ID NO: 889 | ATGATCTTAGCAAT | SOS2 | | | 6.6 | 3.1 | -9.4 |
| SEQ ID NO: 890 | CTGATGATCTTAGC | SOS2 | | | 20.1 | 2.0 | -10.8 |
| SEQ ID NO: 891 | TGGCTTACTCCGTT | SOS2 | | | 22.1 | 1.5 | 7.1 |
| SEQ ID NO: 892 | TAATATTATGGCTT | SOS2 | | | 22.5 | 8.5 | 0.2 |
| SEQ ID NO: 893 | CATTCAATTGGTGG | SOS2 | | | 38.0 | 8.4 | -1.4 |
| SEQ ID NO: 894 | ATGAAGTGTCATGA | SOS2 | | | 28.8 | 2.0 | 0.0 |
| SEQ ID NO: 895 | CTGACGTGCAATTT | SOS2 | | | 28.3 | 11.0 | 1.1 |
| SEQ ID NO: 896 | TCAGCTGACGTGCA | SOS2 | | | 38.8 | 7.4 | 2.4 |
| SEQ ID NO: 897 | TGTCAGCTGACGTG | SOS2 | | | 33.1 | 2.2 | -2.3 |
| SEQ ID NO: 898 | GACGGTTGAACTTT | SOS2 | | | 42.5 | 2.6 | 5.8 |
| SEQ ID NO: 899 | ACAAGTTCAGACGG | SOS2 | | | 36.4 | 16.9 | -14.6 |
| SEQ ID NO: 900 | ATGGCGAATCATTT | SOS2 | | | 36.6 | 6.7 | -2.8 |
| SEQ ID NO: 901 | TGGTATGGCGAATC | SOS2 | | | 34.4 | 3.3 | -0.3 |
| SEQ ID NO: 902 | TGTGGTATGGCGAA | SOS2 | | | 25.5 | 7.2 | 26.7 |
| SEQ ID NO: 903 | AGATTTGTGGTATG | SOS2 | | | 24.1 | 5.9 | 6.4 |
| SEQ ID NO: 904 | GCCACCCGTTCTTC | SOS2 | | | 22.0 | 10.0 | 1.7 |
| SEQ ID NO: 905 | GCAGAATTTCTATA | SOS2 | | | 17.8 | 8.3 | -4.2 |
| SEQ ID NO: 906 | GCCATTGAAATTAT | SOS2 | | | 29.3 | 4.0 | 2.0 |
| SEQ ID NO: 907 | GAATTTACTGCACT | SOS2 | | | 29.4 | 14.3 | 19.0 |
| SEQ ID NO: 908 | GACACTGAATTTAC | SOS2 | | | 34.0 | 6.9 | 16.9 |
| SEQ ID NO: 909 | TGACTTAATTCCAC | SOS2 | | | 29.8 | 2.5 | 10.0 |
| SEQ ID NO: 910 | GTTTTACTAGGTAT | SOS2 | | | 26.4 | 5.4 | 11.1 |
| SEQ ID NO: 911 | AAGTTTTACTAGGT | SOS2 | | | 29.8 | 1.4 | 8.5 |
| SEQ ID NO: 912 | TTCTTCGGTCTTCA | SOS2 | 7.3 | 10.5 | -0.1 | 11.0 | 45.0 |
| SEQ ID NO: 913 | TCCCTTCTTCGGTC | SOS2 | | | 1.5 | 8.4 | 0.6 |
| SEQ ID NO: 914 | ATCATTATTCCCTT | SOS2 | 29.3 | 1.8 | 32.6 | 8.1 | 37.5 |
| SEQ ID NO: 915 | AATCTTTCCCTTTC | SOS2 | 32.9 | 1.3 | 14.4 | 4.6 | 33.8 |
| SEQ ID NO: 916 | CCGTAAACAGTAAG | SOS2 | | | 4.5 | 7.0 | 20.3 |
| SEQ ID NO: 917 | TCTATCCGTAAACA | SOS2 | | | -3.7 | 3.4 | 12.8 |
| SEQ ID NO: 918 | CTGGTTCTATCCGT | SOS2 | | | -1.9 | 7.9 | 6.8 |
| SEQ ID NO: 919 | CCTCATATCTGGTT | SOS2 | | | 2.1 | 3.2 | 2.4 |
| SEQ ID NO: 920 | GGGTTAAGGTTTTC | SOS2 | −2.8 | 5.6 | 23.4 | 1.0 | 34.6 |
| SEQ ID NO: 921 | TCCCATGGGGTTAA | SOS2 | | | 19.2 | 16.2 | 3.0 |
| SEQ ID NO: 922 | TTTCGAGGTTCAAT | SOS2 | | | -24.9 | 1.3 | -1.2 |
| SEQ ID NO: 923 | GGTGGCTGTTTGCA | SOS2 | | | 23.4 | 0.8 | 25.9 |
| SEQ ID NO: 924 | TCGAGGTGGCTGTT | SOS2 | | | 27.8 | 8.7 | 3.4 |
| SEQ ID NO: 925 | GTTGATTTCCTAGG | SOS2 | | | 24.0 | 9.3 | 15.0 |
| SEQ ID NO: 926 | TGTTAGGCCTTATT | SOS2 | | | -13.3 | 8.4 | 5.3 |
| SEQ ID NO: 927 | TGTCGGCCTGTGTT | SOS2 | | | 9.1 | 12.2 | 10.9 |
| SEQ ID NO: 928 | CATGTCGGCCTGTG | SOS2 | | | -12.0 | 14.6 | 3.2 |
| SEQ ID NO: 929 | GTAGAGCCATGTCG | SOS2 | | | 31.3 | 12.9 | -3.5 |
| SEQ ID NO: 930 | GTTGGGTGACCTCG | SOS2 | | | 23.0 | 2.9 | 14.8 |
| SEQ ID NO: 931 | CTCTTTCTAATGGT | SOS2 | | | 15.5 | 12.7 | 6.9 |
| SEQ ID NO: 932 | CCGACTAAAGCTTA | SOS2 | 10.6 | 2.9 | 30.8 | 9.1 | 9.1 |
| SEQ ID NO: 933 | CAATCCGACTAAAG | SOS2 | | | 2.2 | 12.8 | 3.7 |
| SEQ ID NO: 934 | AGCAATCCGACTAA | SOS2 | | | -2.2 | 13.3 | 9.3 |
| SEQ ID NO: 935 | TTCAGCAATCCGAC | SOS2 | | | 4.6 | 0.7 | 22.3 |
| SEQ ID NO: 936 | TGTTGATTCCAGCT | SOS2 | | | 13.5 | 10.8 | 9.7 |
| SEQ ID NO: 937 | CTGACACTGTTGAT | SOS2 | | | 21.6 | 2.7 | -9.5 |
| SEQ ID NO: 938 | TACACTAAGGTCTG | SOS2 | | | 24.7 | 14.1 | -4.8 |
| SEQ ID NO: 939 | GGAGCAAAGATGCT | SOS2 | | | 9.7 | 4.7 | 15.1 |
| SEQ ID NO: 940 | GGGCTCTTCACTTA | SOS2 | | | 0.9 | 2.1 | 14.0 |
| SEQ ID NO: 941 | AAACTTTTTTCGAG | SOS2 | | | -4.7 | 0.6 | 11.5 |
| SEQ ID NO: 942 | GGTGGAATAGCAGG | SOS2 | | | 21.1 | 9.8 | 14.3 |
| SEQ ID NO: 943 | GAGGCTGTCTCGGT | SOS2 | | | 19.9 | 3.0 | 5.5 |
| SEQ ID NO: 944 | GGTGGACTATGCAG | SOS2 | | | 19.5 | 5.9 | 6.7 |
| SEQ ID NO: 945 | GTGGCTGAAGATTA | SOS2 | | | 11.4 | 5.7 | 6.8 |
| SEQ ID NO: 946 | CACGTACTAATGTC | SOS2 | | | 6.2 | 6.9 | 12.8 |
| SEQ ID NO: 947 | GCTTGGCGAATTTG | SOS2 | | | 17.0 | 4.2 | 14.4 |
| SEQ ID NO: 948 | CGCGGTACCCTTGG | SOS2 | | | 27.3 | 3.0 | 15.6 |
| SEQ ID NO: 949 | TCGACGCGGTACCC | SOS2 | | | 28.3 | 3.6 | 12.3 |
| SEQ ID NO: 950 | CATCGACGCGGTAC | SOS2 | | | 15.8 | 10.7 | 14.6 |
| SEQ ID NO: 951 | AGCATCGACGCGGT | SOS2 | 10.4 | 11.1 | 30.3 | 7.6 | 16.0 |
| SEQ ID NO: 952 | CTAGAACTGAGCAC | SOS2 | 9.6 | 6.5 | 37.4 | 0.6 | 21.4 |
| SEQ ID NO: 953 | GATTATTCTGACTA | SOS2 | | | 14.4 | 11.3 | 14.8 |
| SEQ ID NO: 954 | GGATGAGCAAGATT | SOS2 | | | 28.1 | 2.5 | 15.3 |
| SEQ ID NO: 955 | AGATGAGGGCTTGA | SOS2 | 15.0 | 0.7 | 14.7 | 3.0 | 28.6 |
| SEQ ID NO: 956 | TTGGCAGATGAGGG | SOS2 | | | 31.3 | 0.1 | 12.4 |
| SEQ ID NO: 957 | GGCAGTTTTGGCAG | SOS2 | | | 31.1 | 15.4 | 6.6 |
| SEQ ID NO: 958 | TTGTAAGTCTTTGG | SOS2 | | | 21.3 | 8.4 | 15.8 |
| SEQ ID NO: 959 | CCGTTTGTAAGTCT | SOS2 | | | 17.1 | 16.1 | 6.4 |
| SEQ ID NO: 960 | GCTCCCGTTTGTAA | SOS2 | | | -9.9 | 4.6 | 6.0 |
| SEQ ID NO: 961 | GTACAATGGGGGGT | SOS2 | | | 24.3 | 0.4 | -1.2 |
| SEQ ID NO: 962 | AAAGGCAGTCTGTA | SOS2 | | | 0.6 | 0.5 | 6.2 |
| SEQ ID NO: 963 | TCTAGCAAAGGCAG | SOS2 | | | 37.0 | 4.3 | 1.7 |
| SEQ ID NO: 964 | GCATTTTCTAGCAA | SOS2 | | | 4.9 | 9.5 | 3.4 |
| SEQ ID NO: 965 | TGGCTAAGGTCATT | SOS2 | | | 19.9 | 6.7 | 9.3 |
| SEQ ID NO: 966 | ATACCATTCCAGTG | SOS2 | | | 19.8 | 16.1 | 4.8 |
| SEQ ID NO: 967 | TGTAAGAGCATTAT | SOS2 | | | 25.3 | 9.9 | 5.8 |
| SEQ ID NO: 968 | AAGCTTATTTGATC | SOS2 | | | -6.0 | 6.7 | 3.8 |
| SEQ ID NO: 969 | GATAATGGTGAAGG | SOS2 | | | 14.9 | 13.8 | 3.5 |
| SEQ ID NO: 970 | ATACTGGCCTTTTG | SOS2 | 34.8 | 5.0 | 36.6 | 7.3 | 15.1 |
| SEQ ID NO: 971 | ATCATGTCACTTTA | SOS2 | 21.5 | 2.3 | 32.1 | 6.3 | 43.4 |
| SEQ ID NO: 972 | TGCAATCATGTCAC | SOS2 | | | 25.4 | 7.7 | 4.0 |
| SEQ ID NO: 973 | CATCTATTACTCAA | SOS2 | | | 20.5 | 2.0 | 6.5 |
| SEQ ID NO: 974 | TGGTCAAAGAGGTT | SOS2 | | | 30.8 | 1.4 | 18.4 |
| SEQ ID NO: 975 | GTTGGTGTTATTCA | SOS2 | | | 14.4 | 0.7 | 13.6 |
| SEQ ID NO: 976 | CAGTTGGTGTTATT | SOS2 | | | 3.5 | 1.9 | 22.2 |
| SEQ ID NO: 977 | AAGAACAGTTGGTG | SOS2 | | | 36.1 | 4.8 | 7.7 |
| SEQ ID NO: 978 | ATCATTGGGTTATG | SOS2 | | | 17.9 | 5.0 | 0.0 |
| SEQ ID NO: 979 | AAAGGCACATCTGA | SOS2 | | | 10.5 | 6.7 | -0.7 |
| SEQ ID NO: 980 | ATATAGGTTATTAG | SOS2 | | | -1.9 | 15.8 | -2.4 |
| SEQ ID NO: 981 | TATAGGCCTCAAGT | SOS2 | | | 21.3 | 6.7 | -1.9 |
| SEQ ID NO: 982 | GATTGTATTATAGG | SOS2 | | | 27.9 | 3.2 | -3.2 |
| SEQ ID NO: 983 | GCAGATTGTATTAT | SOS2 | | | 16.8 | 15.5 | 10.1 |
| SEQ ID NO: 984 | AGTAGCAGATTGTA | SOS2 | | | 25.3 | 6.8 | 20.3 |
| SEQ ID NO: 985 | GTAAAGTAGCAGAT | SOS2 | | | 33.6 | 9.1 | 7.0 |
| SEQ ID NO: 986 | TTTATGAATTAGTA | SOS2 | | | 15.6 | 9.0 | -11.8 |
| SEQ ID NO: 987 | CAGTTGGTTCCTAT | SOS2 | | | 12.7 | 10.8 | -5.6 |
| SEQ ID NO: 988 | CAAGGCAGTTGGTT | SOS2 | | | 19.8 | 5.7 | 4.7 |
| SEQ ID NO: 989 | TGAAGGCAAGGCAG | SOS2 | | | 24.4 | 4.4 | 19.2 |
| SEQ ID NO: 990 | ACTAGGTCTTGAAG | SOS2 | | | 21.1 | 2.3 | -5.4 |
| SEQ ID NO: 991 | AAGTTACTAGGTCT | SOS2 | | | 31.3 | 2.0 | 6.2 |
| SEQ ID NO: 992 | ACGAGATTTATAGA | SOS2 | | | 34.5 | 10.2 | 7.0 |
| SEQ ID NO: 993 | AACACGAGATTTAT | SOS2 | | | 28.4 | 1.2 | 10.2 |
| SEQ ID NO: 994 | GTTAACACGAGATT | SOS2 | | | 42.8 | 0.3 | 0.5 |
| SEQ ID NO: 995 | ACTTATCTGATTAA | SOS2 | | | 7.6 | 5.2 | 3.4 |
| SEQ ID NO: 996 | GTAAATAAACAACG | SOS2 | | | 0.0 | 0.0 | 7.0 |
| SEQ ID NO: 997 | TTATTAATCTTGTT | SOS2 | | | 1.6 | 8.7 | 2.9 |
| SEQ ID NO: 998 | ACTGGGGTGATTTA | SOS2 | | | 0.7 | 7.3 | 10.1 |
| SEQ ID NO: 999 | ATTCATGTATATCT | SOS1 | 15.8 | 6.3 | 37.2 | 0.1 | 1.6 |
| SEQ ID NO: 1000 | GTTCATGTATATCT | SOS1 | 63.7 | 3.0 | 30.7 | 9.6 | 34.3 |
| SEQ ID NO: 1001 | AATTCATGTATATC | SOS1 | 1.1 | 4.8 | 6.4 | 11.1 | -1.6 |
| SEQ ID NO: 1002 | AGTTCATGTATATC | SOS1 | 24.1 | 10.6 | -3.5 | 0.6 | 2.6 |

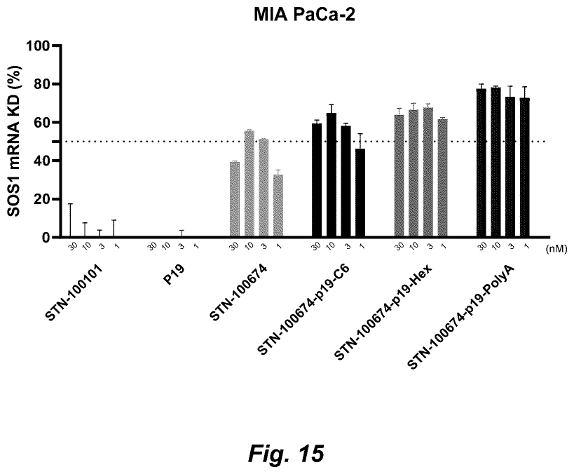

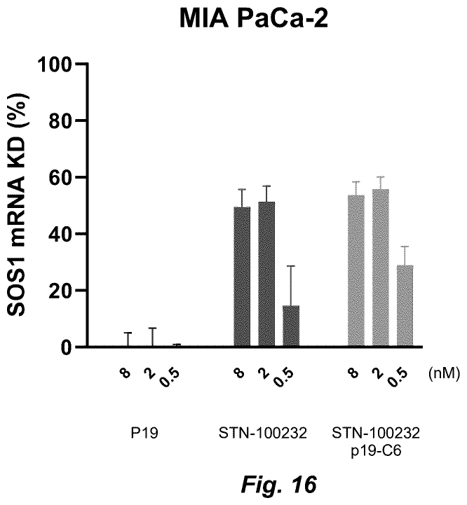

P19 aptamer was conjugated with ASO 674 using three different linkers (HEG spacer, C6 propyl spacer, and oligoA₁₂). ASO 674 is a fully phosphorothioate-linked 3-8-3 gapmer. Each of the conjugates were transfected at increasing nanomolar concentrations into MIA PaCa-2 (ATCC) cells. The SOS1 and SOS2 mRNA knockdown data were recorded as mRNA knockdown percentage. FIG. 15 shows P19 aptamer-conjugated ASO 674 preserves the SOS1-specific, on target activity of all P19-conjugates using three different linkers (HEG spacer, C3 propyl spacer, and oligoA₁₂). Likewise, FIG. 16 shows that a P19 aptamer-conjugated ASO 232 (a fully phosphorothioate-linked 3-8-3 gapmer) preserves the SOS1-specific, on target activity of the P19-conjugate using a 2x C3 propyl spacer when compared to antisense oligonucleotide alone.

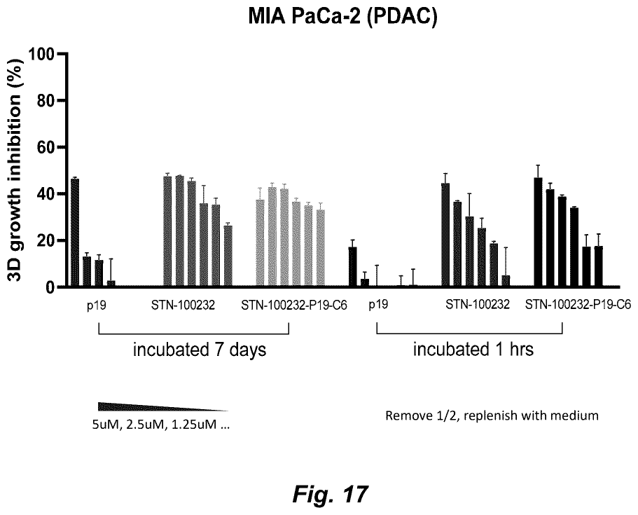

FIG. 17 illustrates 3D cell proliferation inhibition due to inhibiting expression of SOS1 mRNA by contacting the MIA PaCa-2 cells with a P19 aptamer-conjugate (e.g., ASO 232) using C6 linker. In FIG. 17, MIA PaCa-2 cells contacted P19 aptamer-conjugates for 7 days, or for 1 hour then were washed out and replenished with medium. The washout experiment that the P19 aptamer-conjugate (ASO 232) with a C6 propyl spacer showed increased 3D cell proliferation inhibition compared to ASO 232 alone.

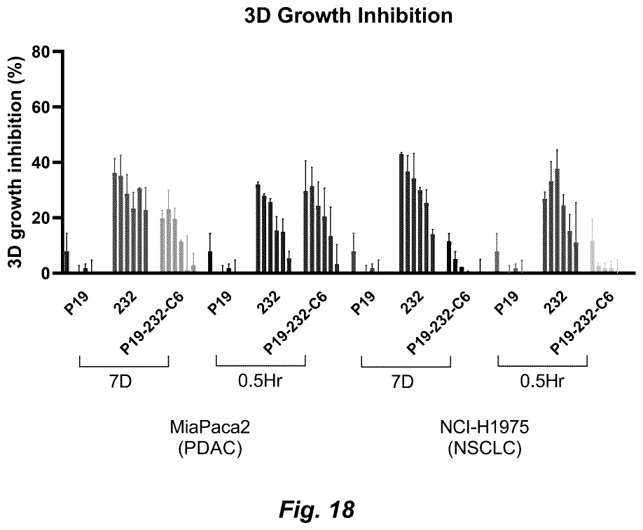

FIG. 18 illustrates 3D cell proliferation inhibition due to inhibiting expression of SOS1 mRNA in MIA PaCa-2 (PDAC) and NCI-H1975 (NSCLC, ATCC CRL-5908) cells with the P19 aptamer-ASO 232 conjugate (C6 linker) at 7 days and with 30 minutes followed by washout. In FIG. 18, the results indicate 3D cell proliferation inhibition by ASO 232 to both MIA PaCa-2 (PDAC) and NCI-H1975 (NSCLC, ATCC CRL-5908) cells. The P19 aptamer-conjugate (ASO 232) with a C6 propyl spacer only showed increased 3D cell proliferation inhibition in MIA PaCa-2 (PDAC), but not NCI-H1975 (NSCLC, ATCC CRL-5908) cells. These results support P19 aptamer specificity for MIA PaCa-2 (PDAC) cells.

The invention claimed is:

1. A composition comprising:
an aptamer comprising a nucleotide sequence that targets accumulation of the composition to pancreatic cancer cells, and
an antisense oligonucleotide that inhibits the expression of an mRNA associated with the KRAS-RAF-MEK-ERK signaling pathway or RTK-RAS-ERK signaling pathway in pancreatic cancer cells.

2. The composition of claim 1, wherein the aptamer comprises the nucleotide sequence GAAUGCCC (SEQ ID NO: 1003), CUCAAUGGCGAAUGCCCGCCUAA UAGGG (SEQ ID NO: 1004), GGGAGACAAGAAUAAACGCUCAAUGGCGAAUG CCCGCCUAAUAGGGCGUUAUGACUUGUUGAGUUCGACAGGAGGCUCACAACAGGC (SEQ ID NO: 1005) or a derivative thereof.

3.-4. (canceled)

5. The composition of claim 1, wherein the aptamer nucleotide sequence is chemically modified.

6. (canceled)

7. The composition of claim 1, wherein the antisense oligonucleotide targets KRAS mRNA.

8. The composition of claim 7, wherein the antisense oligonucleotide comprises at least 8, or at least 10, or at least 12 contiguous nucleotides of an oligonucleotide from any one of Table 1 to 4.

9.-10. (canceled)

11. The composition of claim 7, wherein the antisense oligonucleotide targets KRAS mRNA encoding a mutant KRAS.

12.-13. (canceled)

14. The composition of claim 1, wherein the antisense oligonucleotide targets SOS1 and/or SOS2 mRNA.

15. The composition of claim 14, wherein the antisense oligonucleotide comprises at least 8, or at least 10, or at least 12 contiguous nucleotides of a sequence from Table 13.

16.-18. (canceled)

19. The composition of claim 1, wherein the antisense oligonucleotide has a stretch of at least 6 DNA nucleotides sufficient to recruit RNaseH.

20. (canceled)

21. The composition of claim 19, wherein one or more DNA nucleotides comprise a 2′ chemical modification independently selected from 2′-Fluoro, 2′-Methyl, and 2′-Ethyl.

22. The composition of claim 19, wherein the antisense oligonucleotide is a gapmer having a 5′ and a 3′ segment, each of the 5′ and 3′ segments being from 2 to 6 nucleotides or from 2 to 4 nucleotides, and where the 5′ and 3′ segments do not contain DNA nucleotides.

23. (canceled)

24. The composition of claim 22, wherein one or more nucleotides of the 5′ segment and the 3′ segment comprise 2′-O substituents, optionally where all of the nucleotides of the 5′ segment and the 3′ segment comprise 2′-O substituents.

25.-26. (canceled)

27. The composition of claim 1, wherein the antisense oligonucleotide has a modified backbone.

28. The composition of claim 27, wherein the antisense oligonucleotide and/or the aptamer comprises one or more phosphorothioate or phosphorodithioate nucleotides.

29. (canceled)

30. The composition of claim 1, wherein cytidine nucleobases in the antisense oligonucleotide and/or the aptamer are 5-methyl cytidine.

31. The composition of claim 1, wherein the antisense oligonucleotide hybridizes to its target sequence with a Tm of at least about 35° C., or at least about 40° C., or at least about 45° C., or at least about 50° C.

32. (canceled)

33. The composition of claim 1, wherein the aptamer and the antisense oligonucleotide are linked directly or indirectly through a linker.

34.-43. (canceled)

44. The composition of claim 1, wherein the antisense oligonucleotide is encapsulated in a particle, and the aptamer is presented on the surface of the particle.

45. The composition of claim 44, wherein the particle is a liposome, polymeric nanoparticle, or lipid nanoparticle.

46. A method for treating a subject having pancreatic cancer, comprising administering an effective amount of the composition of any one of claims 1 to 45 to the subject.

47.-50. (canceled)


SEQUENCE LISTING (1004 sequences)

SEQ ID NO: 1 - DNA, 14 bp, synthetic construct
  source 1..14: mol_type = other DNA
tcctctattgttgg

SEQ ID NO: 2 - DNA, 14 bp, synthetic construct
  source 1..14: mol_type = other DNA
atcctctattgttg

SEQ ID NO: 3 - DNA, 14 bp, synthetic construct
  source 1..14: mol_type = other DNA
aatcctctattgtt

SEQ ID NO: 4 - DNA, 14 bp, synthetic construct
  source 1..14: mol_type = other DNA
gaatcctctattgt

SEQ ID NO: 5 - DNA, 14 bp, synthetic construct
  source 1..14: mol_type = other DNA
ggaatcctctattg

SEQ ID NO: 6 - DNA, 14 bp, synthetic construct
  source 1..14: mol_type = other DNA
aggaatcctctatt

SEQ ID NO: 7 - DNA, 14 bp, synthetic construct
  source 1..14: mol_type = other DNA
taggaatcctctat

SEQ ID NO: 8 - DNA, 14 bp, synthetic construct
  source 1..14: mol_type = other DNA
gtaggaatcctcta

SEQ ID NO: 9 - DNA, 14 bp, synthetic construct
  source 1..14: mol_type = other DNA
tgtaggaatcctct

SEQ ID NO: 10 - DNA, 14 bp, synthetic construct
  source 1..14: mol_type = other DNA
ctgtaggaatcctc

SEQ ID NO: 11 - DNA, 14 bp, synthetic construct
  source 1..14: mol_type = other DNA
cctgtaggaatcct

SEQ ID NO: 12 - DNA, 14 bp, synthetic construct
  source 1..14: mol_type = other DNA
tcctgtaggaatcc

SEQ ID NO: 13 - DNA, 14 bp, synthetic construct
  source 1..14: mol_type = other DNA
ttcctgtaggaatc

SEQ ID NO: 14 - DNA, 14 bp, synthetic construct
  source 1..14: mol_type = other DNA
cttcctgtaggaat

SEQ ID NO: 15 - DNA, 14 bp, synthetic construct
  source 1..14: mol_type = other DNA
gcttcctgtaggaa

SEQ ID NO: 16 - DNA, 14 bp, synthetic construct
  source 1..14: mol_type = other DNA
tgcttcctgtagga

SEQ ID NO: 17 - DNA, 14 bp, synthetic construct
  source 1..14: mol_type = other DNA
ttgcttcctgtagg

SEQ ID NO: 18 - DNA, 14 bp, synthetic construct
  source 1..14: mol_type = other DNA
cttgcttcctgtag

SEQ ID NO: 19 - DNA, 14 bp, synthetic construct
  source 1..14: mol_type = other DNA
acttgcttcctgta

SEQ ID NO: 20 - DNA, 14 bp, synthetic construct
  source 1..14: mol_type = other DNA
tacttgcttcctgt

SEQ ID NO: 21 - DNA, 14 bp, synthetic construct
  source 1..14: mol_type = other DNA
ctacttgcttcctg

SEQ ID NO: 22 - DNA, 14 bp, synthetic construct
  source 1..14: mol_type = other DNA
actacttgcttcct

SEQ ID NO: 23 - DNA, 14 bp, synthetic construct
  source 1..14: mol_type = other DNA
tactacttgcttcc

SEQ ID NO: 24 - DNA, 14 bp, synthetic construct
  source 1..14: mol_type = other DNA
ttactacttgcttc

SEQ ID NO: 25 - DNA, 14 bp, synthetic construct
  source 1..14: mol_type = other DNA
attactacttgctt

SEQ ID NO: 26 - DNA, 14 bp, synthetic construct
  source 1..14: mol_type = other DNA
aattactacttgct

SEQ ID NO: 27 - DNA, 14 bp, synthetic construct
  source 1..14: mol_type = other DNA
caattactacttgc

SEQ ID NO: 28 - DNA, 14 bp, synthetic construct
  source 1..14: mol_type = other DNA
tcaattactacttg

SEQ ID NO: 29 - DNA, 14 bp, synthetic construct
  source 1..14: mol_type = other DNA
atcaattactactt

SEQ ID NO: 30 - DNA, 14 bp, synthetic construct
  source 1..14: mol_type = other DNA
catcaattactact

SEQ ID NO: 31 - DNA, 14 bp, synthetic construct
  source 1..14: mol_type = other DNA
ccatcaattactac

SEQ ID NO: 32 - DNA, 14 bp, synthetic construct
  source 1..14: mol_type = other DNA
tccatcaattacta

SEQ ID NO: 33 - DNA, 14 bp, synthetic construct
  source 1..14: mol_type = other DNA
ctccatcaattact

SEQ ID NO: 34 - DNA, 14 bp, synthetic construct
  source 1..14: mol_type = other DNA
tctccatcaattac

SEQ ID NO: 35 - DNA, 14 bp, synthetic construct
  source 1..14: mol_type = other DNA
ttctccatcaatta

SEQ ID NO: 36 - DNA, 14 bp, synthetic construct
  source 1..14: mol_type = other DNA
tttctccatcaatt

SEQ ID NO: 37 - DNA, 14 bp, synthetic construct
  source 1..14: mol_type = other DNA
gtttctccatcaat

SEQ ID NO: 38 - DNA, 14 bp, synthetic construct
  source 1..14: mol_type = other DNA
ggtttctccatcaa

SEQ ID NO: 39 - DNA, 14 bp, synthetic construct
  source 1..14: mol_type = other DNA
aggtttctccatca

SEQ ID NO: 40 - DNA, 14 bp, synthetic construct
  source 1..14: mol_type = other DNA
caggtttctccatc

SEQ ID NO: 41 - DNA, 14 bp, synthetic construct
  source 1..14: mol_type = other DNA
acaggtttctccat

SEQ ID NO: 42 - DNA, 14 bp, synthetic construct
  source 1..14: mol_type = other DNA
gacaggtttctcca

SEQ ID NO: 43 - DNA, 14 bp, synthetic construct
  source 1..14: mol_type = other DNA
agacaggtttctcc

SEQ ID NO: 44 - DNA, 14 bp, synthetic construct
  source 1..14: mol_type = other DNA
gagacaggtttctc

SEQ ID NO: 45 - DNA, 14 bp, synthetic construct
  source 1..14: mol_type = other DNA
agagacaggtttct

SEQ ID NO: 46 - DNA, 14 bp, synthetic construct
  source 1..14: mol_type = other DNA
aagagacaggtttc

SEQ ID NO: 47 - DNA, 14 bp, synthetic construct
  source 1..14: mol_type = other DNA
caagagacaggttt

SEQ ID NO: 48 - DNA, 14 bp, synthetic construct
  source 1..14: mol_type = other DNA
ccaagagacaggtt

SEQ ID NO: 49 - DNA, 14 bp, synthetic construct
  source 1..14: mol_type = other DNA
tccaagagacaggt

SEQ ID NO: 50 - DNA, 14 bp, synthetic construct
  source 1..14: mol_type = other DNA
atccaagagacagg

SEQ ID NO: 51 - DNA, 14 bp, synthetic construct
  source 1..14: mol_type = other DNA
tatccaagagacag

SEQ ID NO: 52 - DNA, 14 bp, synthetic construct
  source 1..14: mol_type = other DNA
atatccaagagaca

SEQ ID NO: 53 - DNA, 14 bp, synthetic construct
  source 1..14: mol_type = other DNA
aatatccaagagac

SEQ ID NO: 54 - DNA, 14 bp, synthetic construct
  source 1..14: mol_type = other DNA
gaatatccaagaga

SEQ ID NO: 55 - DNA, 14 bp, synthetic construct
  source 1..14: mol_type = other DNA
agaatatccaagag

SEQ ID NO: 56 - DNA, 14 bp, synthetic construct
  source 1..14: mol_type = other DNA
gagaatatccaaga

SEQ ID NO: 57 - DNA, 14 bp, synthetic construct
  source 1..14: mol_type = other DNA
cgagaatatccaag

SEQ ID NO: 58 - DNA, 14 bp, synthetic construct
  source 1..14: mol_type = other DNA
tcgagaatatccaa

SEQ ID NO: 59 - DNA, 14 bp, synthetic construct
  source 1..14: mol_type = other DNA
gtcgagaatatcca

SEQ ID NO: 60 - DNA, 14 bp, synthetic construct
  source 1..14: mol_type = other DNA
tgtcgagaatatcc

SEQ ID NO: 61 - DNA, 14 bp, synthetic construct
  source 1..14: mol_type = other DNA
gtgtcgagaatatc

SEQ ID NO: 62 - DNA, 14 bp, synthetic construct
  source 1..14: mol_type = other DNA
tgtgtcgagaatat

SEQ ID NO: 63 - DNA, 14 bp, synthetic construct
  source 1..14: mol_type = other DNA
ctgtgtcgagaata

SEQ ID NO: 64 - DNA, 14 bp, synthetic construct
  source 1..14: mol_type = other DNA
gctgtgtcgagaat

SEQ ID NO: 65 - DNA, 14 bp, synthetic construct
  source 1..14: mol_type = other DNA
tgctgtgtcgagaa

SEQ ID NO: 66 - DNA, 14 bp, synthetic construct
  source 1..14: mol_type = other DNA
ctgctgtgtcgaga

SEQ ID NO: 67 - DNA, 14 bp, synthetic construct
  source 1..14: mol_type = other DNA
cctgctgtgtcgag

SEQ ID NO: 68 - DNA, 14 bp, synthetic construct
  source 1..14: mol_type = other DNA
acctgctgtgtcga

SEQ ID NO: 69 - DNA, 14 bp, synthetic construct
  source 1..14: mol_type = other DNA
gacctgctgtgtcg

SEQ ID NO: 70 - DNA, 14 bp, synthetic construct
  source 1..14: mol_type = other DNA
tgacctgctgtgtc

SEQ ID NO: 71 - DNA, 14 bp, synthetic construct
  source 1..14: mol_type = other DNA
ttgacctgctgtgt

SEQ ID NO: 72 - DNA, 14 bp, synthetic construct
  source 1..14: mol_type = other DNA
cttgacctgctgtg

SEQ ID NO: 73 - DNA, 14 bp, synthetic construct
  source 1..14: mol_type = other DNA
tcttgacctgctgt

SEQ ID NO: 74 - DNA, 14 bp, synthetic construct
  source 1..14: mol_type = other DNA
ctcttgacctgctg

SEQ ID NO: 75 - DNA, 14 bp, synthetic construct
  source 1..14: mol_type = other DNA
cctcttgacctgct

SEQ ID NO: 76 - DNA, 14 bp, synthetic construct
  source 1..14: mol_type = other DNA
tcctcttgacctgc

SEQ ID NO: 77 - DNA, 14 bp, synthetic construct
  source 1..14: mol_type = other DNA
ctcctcttgacctg

SEQ ID NO: 78 - DNA, 14 bp, synthetic construct
  source 1..14: mol_type = other DNA
actcctcttgacct

SEQ ID NO: 79 - DNA, 14 bp, synthetic construct
  source 1..14: mol_type = other DNA
tactcctcttgacc

SEQ ID NO: 80 - DNA, 14 bp, synthetic construct
  source 1..14: mol_type = other DNA
gtactcctcttgac

SEQ ID NO: 81 - DNA, 14 bp, synthetic construct
  source 1..14: mol_type = other DNA
tgtactcctcttga

SEQ ID NO: 82 - DNA, 14 bp, synthetic construct
  source 1..14: mol_type = other DNA
ctgtactcctcttg

SEQ ID NO: 83 - DNA, 14 bp, synthetic construct
  source 1..14: mol_type = other DNA
actgtactcctctt

SEQ ID NO: 84 - DNA, 14 bp, synthetic construct
  source 1..14: mol_type = other DNA
cactgtactcctct

SEQ ID NO: 85 - DNA, 14 bp, synthetic construct
  source 1..14: mol_type = other DNA
gcactgtactcctc

SEQ ID NO: 86 - DNA, 14 bp, synthetic construct
  source 1..14: mol_type = other DNA
tgcactgtactcct

SEQ ID NO: 87 - DNA, 14 bp, synthetic construct
  source 1..14: mol_type = other DNA
ttgcactgtactcc

SEQ ID NO: 88 - DNA, 14 bp, synthetic construct
  source 1..14: mol_type = other DNA
attgcactgtactc

SEQ ID NO: 89 - DNA, 14 bp, synthetic construct
  source 1..14: mol_type = other DNA
cattgcactgtact

SEQ ID NO: 90 - DNA, 14 bp, synthetic construct
  source 1..14: mol_type = other DNA
tcattgcactgtac

SEQ ID NO: 91 - DNA, 14 bp, synthetic construct
  source 1..14: mol_type = other DNA
ctcattgcactgta

SEQ ID NO: 92 - DNA, 14 bp, synthetic construct
  source 1..14: mol_type = other DNA
cctcattgcactgt

SEQ ID NO: 93 - DNA, 14 bp, synthetic construct
  source 1..14: mol_type = other DNA
ccctcattgcactg

SEQ ID NO: 94 - DNA, 14 bp, synthetic construct
  source 1..14: mol_type = other DNA
tccctcattgcact

SEQ ID NO: 95 - DNA, 14 bp, synthetic construct
  source 1..14: mol_type = other DNA
gtccctcattgcac

SEQ ID NO: 96 - DNA, 14 bp, synthetic construct
  source 1..14: mol_type = other DNA
ggtccctcattgca

SEQ ID NO: 97 - DNA, 14 bp, synthetic construct
  source 1..14: mol_type = other DNA
tggtccctcattgc

SEQ ID NO: 98 - DNA, 14 bp, synthetic construct
  source 1..14: mol_type = other DNA
ctggtccctcattg

SEQ ID NO: 99 - DNA, 14 bp, synthetic construct
  source 1..14: mol_type = other DNA
actggtccctcatt

SEQ ID NO: 100 - DNA, 14 bp, synthetic construct
  source 1..14: mol_type = other DNA
tactggtccctcat

SEQ ID NO: 101 - DNA, 14 bp, synthetic construct
  source 1..14: mol_type = other DNA
gtactggtccctca

SEQ ID NO: 102 - DNA, 14 bp, synthetic construct
  source 1..14: mol_type = other DNA
tgtactggtccctc

SEQ ID NO: 103 - DNA, 14 bp, synthetic construct
  source 1..14: mol_type = other DNA
atgtactggtccct

SEQ ID NO: 104 - DNA, 14 bp, synthetic construct
  source 1..14: mol_type = other DNA
catgtactggtccc

SEQ ID NO: 105 - DNA, 14 bp, synthetic construct
  source 1..14: mol_type = other DNA
tcatgtactggtcc

SEQ ID NO: 106 - DNA, 14 bp, synthetic construct
  source 1..14: mol_type = other DNA
ctcatgtactggtc

SEQ ID NO: 107 - DNA, 14 bp, synthetic construct
  source 1..14: mol_type = other DNA
cctcatgtactggt

SEQ ID NO: 108 - DNA, 14 bp, synthetic construct
  source 1..14: mol_type = other DNA
tcctcatgtactgg

SEQ ID NO: 109 - DNA, 14 bp, synthetic construct
  source 1..14: mol_type = other DNA
gtcctcatgtactg

SEQ ID NO: 110 - DNA, 14 bp, synthetic construct
  source 1..14: mol_type = other DNA
agtcctcatgtact

SEQ ID NO: 111 - DNA, 14 bp, synthetic construct
  source 1..14: mol_type = other DNA
cagtcctcatgtac

SEQ ID NO: 112 - DNA, 14 bp, synthetic construct
  source 1..14: mol_type = other DNA
ccagtcctcatgta

SEQ ID NO: 113 - DNA, 14 bp, synthetic construct
  source 1..14: mol_type = other DNA
cccagtcctcatgt

SEQ ID NO: 114 - DNA, 14 bp, synthetic construct
  source 1..14: mol_type = other DNA
ccccagtcctcatg

SEQ ID NO: 115 - DNA, 14 bp, synthetic construct
  source 1..14: mol_type = other DNA
tccccagtcctcat

SEQ ID NO: 116 - DNA, 14 bp, synthetic construct
  source 1..14: mol_type = other DNA
ctccccagtcctca

SEQ ID NO: 117 - DNA, 14 bp, synthetic construct
  source 1..14: mol_type = other DNA
cctccccagtcctc

SEQ ID NO: 118 - DNA, 14 bp, synthetic construct
  source 1..14: mol_type = other DNA
ccctccccagtcct

SEQ ID NO: 119 - DNA, 14 bp, synthetic construct
  source 1..14: mol_type = other DNA
gccctccccagtcc

SEQ ID NO: 120 - DNA, 14 bp, synthetic construct
  source 1..14: mol_type = other DNA
agccctccccagtc

SEQ ID NO: 121 - DNA, 14 bp, synthetic construct
  source 1..14: mol_type = other DNA
aagccctccccagt

SEQ ID NO: 122 - DNA, 14 bp, synthetic construct
  source 1..14: mol_type = other DNA
aaagccctccccag

SEQ ID NO: 123 - DNA, 14 bp, synthetic construct
  source 1..14: mol_type = other DNA
gaaagccctcccca

SEQ ID NO: 124 - DNA, 14 bp, synthetic construct
  source 1..14: mol_type = other DNA
agaaagccctcccc

SEQ ID NO: 125 - DNA, 14 bp, synthetic construct
  source 1..14: mol_type = other DNA
aagaaagccctccc

SEQ ID NO: 126 - DNA, 14 bp, synthetic construct
  source 1..14: mol_type = other DNA
aaagaaagccctcc

SEQ ID NO: 127 - DNA, 14 bp, synthetic construct
  source 1..14: mol_type = other DNA
caaagaaagccctc

SEQ ID NO: 128 - DNA, 14 bp, synthetic construct
  source 1..14: mol_type = other DNA
acaaagaaagccct

SEQ ID NO: 129 - DNA, 14 bp, synthetic construct
  source 1..14: mol_type = other DNA
cacaaagaaagccc

SEQ ID NO: 130 - DNA, 14 bp, synthetic construct
  source 1..14: mol_type = other DNA
acacaaagaaagcc

SEQ ID NO: 131 - DNA, 14 bp, synthetic construct
  source 1..14: mol_type = other DNA
tacacaaagaaagc

SEQ ID NO: 132 - DNA, 14 bp, synthetic construct
  source 1..14: mol_type = other DNA
atacacaaagaaag

SEQ ID NO: 133 - DNA, 14 bp, synthetic construct
  source 1..14: mol_type = other DNA
aatacacaaagaaa

SEQ ID NO: 134 - DNA, 14 bp, synthetic construct
  source 1..14: mol_type = other DNA
aaatacacaaagaa

SEQ ID NO: 135 - DNA, 14 bp, synthetic construct
  source 1..14: mol_type = other DNA
caaatacacaaaga

SEQ ID NO: 136 - DNA, 14 bp, synthetic construct
  source 1..14: mol_type = other DNA
gcaaatacacaaag

SEQ ID NO: 137 - DNA, 14 bp, synthetic construct
  source 1..14: mol_type = other DNA
ggcaaatacacaaa

SEQ ID NO: 138 - DNA, 14 bp, synthetic construct
  source 1..14: mol_type = other DNA
tggcaaatacacaa

SEQ ID NO: 139 - DNA, 14 bp, synthetic construct
  source 1..14: mol_type = other DNA
atggcaaatacaca

SEQ ID NO: 140 - DNA, 14 bp, synthetic construct
  source 1..14: mol_type = other DNA
tatggcaaatacac

SEQ ID NO: 141 - DNA, 14 bp, synthetic construct
  source 1..14: mol_type = other DNA
ttatggcaaataca

SEQ ID NO: 142 - DNA, 14 bp, synthetic construct
  source 1..14: mol_type = other DNA
tttatggcaaatac

SEQ ID NO: 143 - DNA, 14 bp, synthetic construct
  source 1..14: mol_type = other DNA
atttatggcaaata

SEQ ID NO: 144 - DNA, 14 bp, synthetic construct
  source 1..14: mol_type = other DNA
tatttatggcaaat

SEQ ID NO: 145 - DNA, 14 bp, synthetic construct
  source 1..14: mol_type = other DNA
ttatttatggcaaa

SEQ ID NO: 146 - DNA, 14 bp, synthetic construct
  source 1..14: mol_type = other DNA
attatttatggcaa

SEQ ID NO: 147 - DNA, 14 bp, synthetic construct
  source 1..14: mol_type = other DNA
tattatttatggca

SEQ ID NO: 148 - DNA, 14 bp, synthetic construct
  source 1..14: mol_type = other DNA
gtattatttatggc

SEQ ID NO: 149 - DNA, 14 bp, synthetic construct
  source 1..14: mol_type = other DNA
agtattatttatgg

SEQ ID NO: 150 - DNA, 14 bp, synthetic construct
  source 1..14: mol_type = other DNA
tagtattatttatg

SEQ ID NO: 151 - DNA, 14 bp, synthetic construct
  source 1..14: mol_type = other DNA
ttagtattatttat

SEQ ID NO: 152 - DNA, 14 bp, synthetic construct
  source 1..14: mol_type = other DNA
tttagtattattta

SEQ ID NO: 153 - DNA, 14 bp, synthetic construct
  source 1..14: mol_type = other DNA
atttagtattattt

SEQ ID NO: 154 - DNA, 14 bp, synthetic construct
  source 1..14: mol_type = other DNA
gatttagtattatt

SEQ ID NO: 155 - DNA, 14 bp, synthetic construct
  source 1..14: mol_type = other DNA
tgatttagtattat

SEQ ID NO: 156 - DNA, 14 bp, synthetic construct
  source 1..14: mol_type = other DNA
atgatttagtatta

SEQ ID NO: 157 - DNA, 14 bp, synthetic construct
  source 1..14: mol_type = other DNA
aatgatttagtatt

SEQ ID NO: 158 - DNA, 14 bp, synthetic construct
  source 1..14: mol_type = other DNA
aaatgatttagtat

SEQ ID NO: 159 - DNA, 14 bp, synthetic construct
  source 1..14: mol_type = other DNA
caaatgatttagta

SEQ ID NO: 160 - DNA, 14 bp, synthetic construct
  source 1..14: mol_type = other DNA
tcaaatgatttagt

SEQ ID NO: 161 - DNA, 14 bp, synthetic construct
  source 1..14: mol_type = other DNA
ttcaaatgatttag

SEQ ID NO: 162 - DNA, 14 bp, synthetic construct
  source 1..14: mol_type = other DNA
cttcaaatgattta

SEQ ID NO: 163 - DNA, 14 bp, synthetic construct
  source 1..14: mol_type = other DNA
tcttcaaatgattt

SEQ ID NO: 164 - DNA, 14 bp, synthetic construct
  source 1..14: mol_type = other DNA
atcttcaaatgatt

SEQ ID NO: 165 - DNA, 14 bp, synthetic construct
  source 1..14: mol_type = other DNA
tatcttcaaatgat

SEQ ID NO: 166 - DNA, 14 bp, synthetic construct
  source 1..14: mol_type = other DNA
atatcttcaaatga

SEQ ID NO: 167 - DNA, 14 bp, synthetic construct
  source 1..14: mol_type = other DNA
aatatcttcaaatg

SEQ ID NO: 168 - DNA, 14 bp, synthetic construct
  source 1..14: mol_type = other DNA
gaatatcttcaaat

SEQ ID NO: 169 - DNA, 14 bp, synthetic construct
  source 1..14: mol_type = other DNA
tgaatatcttcaaa

SEQ ID NO: 170 - DNA, 14 bp, synthetic construct
  source 1..14: mol_type = other DNA
gtgaatatcttcaa

SEQ ID NO: 171 - DNA, 14 bp, synthetic construct
  source 1..14: mol_type = other DNA
ggtgaatatcttca

SEQ ID NO: 172 - DNA, 14 bp, synthetic construct
  source 1..14: mol_type = other DNA
tggtgaatatcttc

SEQ ID NO: 173 - DNA, 14 bp, synthetic construct
  source 1..14: mol_type = other DNA
atggtgaatatctt

SEQ ID NO: 174 - DNA, 14 bp, synthetic construct
  source 1..14: mol_type = other DNA
aatggtgaatatct

SEQ ID NO: 175 - DNA, 14 bp, synthetic construct
  source 1..14: mol_type = other DNA
taatggtgaatatc

SEQ ID NO: 176 - DNA, 14 bp, synthetic construct
  source 1..14: mol_type = other DNA
ataatggtgaatat

SEQ ID NO: 177 - DNA, 14 bp, synthetic construct
  source 1..14: mol_type = other DNA
tataatggtgaata

SEQ ID NO: 178 - DNA, 14 bp, synthetic construct
  source 1..14: mol_type = other DNA
ctataatggtgaat

SEQ ID NO: 179 - DNA, 14 bp, synthetic construct
  source 1..14: mol_type = other DNA
tctataatggtgaa

SEQ ID NO: 180 - DNA, 14 bp, synthetic construct
  source 1..14: mol_type = other DNA
ctctataatggtga

SEQ ID NO: 181 - DNA, 14 bp, synthetic construct
  source 1..14: mol_type = other DNA
tctctataatggtg

SEQ ID NO: 182 - DNA, 14 bp, synthetic construct
  source 1..14: mol_type = other DNA
ttctctataatggt

SEQ ID NO: 183 - DNA, 14 bp, synthetic construct
  source 1..14: mol_type = other DNA
gttctctataatgg

SEQ ID NO: 184 - DNA, 14 bp, synthetic construct
  source 1..14: mol_type = other DNA
tgttctctataatg

SEQ ID NO: 185 - DNA, 14 bp, synthetic construct
  source 1..14: mol_type = other DNA
ttgttctctataat

SEQ ID NO: 186 - DNA, 14 bp, synthetic construct
  source 1..14: mol_type = other DNA
tttgttctctataa

SEQ ID NO: 187 - DNA, 14 bp, synthetic construct
  source 1..14: mol_type = other DNA
atttgttctctata

SEQ ID NO: 188 - DNA, 14 bp, synthetic construct
  source 1..14: mol_type = other DNA
aatttgttctctat

SEQ ID NO: 189 - DNA, 14 bp, synthetic construct
  source 1..14: mol_type = other DNA
taatttgttctcta

SEQ ID NO: 190 - DNA, 14 bp, synthetic construct
  source 1..14: mol_type = other DNA
ttaatttgttctct

SEQ ID NO: 191 - DNA, 14 bp, synthetic construct
  source 1..14: mol_type = other DNA
tttaatttgttctc

SEQ ID NO: 192 - DNA, 14 bp, synthetic construct
  source 1..14: mol_type = other DNA
ttttaatttgttct

SEQ ID NO: 193 - DNA, 14 bp, synthetic construct
  source 1..14: mol_type = other DNA
cttttaatttgttc

SEQ ID NO: 194 - DNA, 14 bp, synthetic construct
  source 1..14: mol_type = other DNA
tcttttaatttgtt

SEQ ID NO: 195 - DNA, 14 bp, synthetic construct
  source 1..14: mol_type = other DNA
ctcttttaatttgt

SEQ ID NO: 196 - DNA, 14 bp, synthetic construct
  source 1..14: mol_type = other DNA
actcttttaatttg

SEQ ID NO: 197 - DNA, 14 bp, synthetic construct
  source 1..14: mol_type = other DNA
aactcttttaattt

SEQ ID NO: 198 - DNA, 14 bp, synthetic construct
  source 1..14: mol_type = other DNA
taactcttttaatt

SEQ ID NO: 199 - DNA, 14 bp, synthetic construct
  source 1..14: mol_type = other DNA
ttaactcttttaat

SEQ ID NO: 200 - DNA, 14 bp, synthetic construct
  source 1..14: mol_type = other DNA
cttaactcttttaa

SEQ ID NO: 201 - DNA, 14 bp, synthetic construct
  source 1..14: mol_type = other DNA
ccttaactctttta

SEQ ID NO: 202 - DNA, 14 bp, synthetic construct
  source 1..14: mol_type = other DNA
tccttaactctttt

SEQ ID NO: 203 - DNA, 14 bp, synthetic construct
  source 1..14: mol_type = other DNA
gtccttaactcttt

SEQ ID NO: 204 - DNA, 14 bp, synthetic construct
  source 1..14: mol_type = other DNA
agtccttaactctt

SEQ ID NO: 205 - DNA, 14 bp, synthetic construct
  source 1..14: mol_type = other DNA
gagtccttaactct

SEQ ID NO: 206 - DNA, 14 bp, synthetic construct
  source 1..14: mol_type = other DNA
agagtccttaactc

SEQ ID NO: 207 - DNA, 14 bp, synthetic construct
  source 1..14: mol_type = other DNA
cagagtccttaact

SEQ ID NO: 208 - DNA, 14 bp, synthetic construct
  source 1..14: mol_type = other DNA
tcagagtccttaac

SEQ ID NO: 209 - DNA, 14 bp, synthetic construct
  source 1..14: mol_type = other DNA
ttcagagtccttaa

SEQ ID NO: 210 - DNA, 14 bp, synthetic construct
  source 1..14: mol_type = other DNA
cttcagagtcctta

SEQ ID NO: 211 - DNA, 14 bp, synthetic construct
  source 1..14: mol_type = other DNA
tcttcagagtcctt

SEQ ID NO: 212 - DNA, 14 bp, synthetic construct
  source 1..14: mol_type = other DNA
atcttcagagtcct

SEQ ID NO: 213 - DNA, 14 bp, synthetic construct
  source 1..14: mol_type = other DNA
catcttcagagtcc

SEQ ID NO: 214 - DNA, 14 bp, synthetic construct
  source 1..14: mol_type = other DNA
acatcttcagagtc

SEQ ID NO: 215 - DNA, 14 bp, synthetic construct
  source 1..14: mol_type = other DNA
tacatcttcagagt

SEQ ID NO: 216 - DNA, 14 bp, synthetic construct
  source 1..14: mol_type = other DNA
gtacatcttcagag

SEQ ID NO: 217 - DNA, 14 bp, synthetic construct
  source 1..14: mol_type = other DNA
ggtacatcttcaga

SEQ ID NO: 218 - DNA, 14 bp, synthetic construct
  source 1..14: mol_type = other DNA
aggtacatcttcag

SEQ ID NO: 219 - DNA, 14 bp, synthetic construct
  source 1..14: mol_type = other DNA
taggtacatcttca

SEQ ID NO: 220 - DNA, 14 bp, synthetic construct
  source 1..14: mol_type = other DNA
ataggtacatcttc

SEQ ID NO: 221 - DNA, 14 bp, synthetic construct
  source 1..14: mol_type = other DNA
cataggtacatctt

SEQ ID NO: 222 - DNA, 14 bp, synthetic construct
  source 1..14: mol_type = other DNA
ccataggtacatct

SEQ ID NO: 223 - DNA, 14 bp, synthetic construct
  source 1..14: mol_type = other DNA
accataggtacatc

SEQ ID NO: 224 - DNA, 14 bp, synthetic construct
  source 1..14: mol_type = other DNA
gaccataggtacat

SEQ ID NO: 225 - DNA, 14 bp, synthetic construct
  source 1..14: mol_type = other DNA
ggaccataggtaca

SEQ ID NO: 226 - DNA, 14 bp, synthetic construct
  source 1..14: mol_type = other DNA
aggaccataggtac

SEQ ID NO: 227 - DNA, 14 bp, synthetic construct
  source 1..14: mol_type = other DNA
taggaccataggta

SEQ ID NO: 228 - DNA, 14 bp, synthetic construct
  source 1..14: mol_type = other DNA
ctaggaccataggt

SEQ ID NO: 229 - DNA, 14 bp, synthetic construct
  source 1..14: mol_type = other DNA
actaggaccatagg

SEQ ID NO: 230 - DNA, 14 bp, synthetic construct
  source 1..14: mol_type = other DNA
tactaggaccatag

SEQ ID NO: 231 - DNA, 14 bp, synthetic construct
  source 1..14: mol_type = other DNA
ctactaggaccata

SEQ ID NO: 232 - DNA, 14 bp, synthetic construct
  source 1..14: mol_type = other DNA
cctactaggaccat

SEQ ID NO: 233 - DNA, 14 bp, synthetic construct
  source 1..14: mol_type = other DNA
tcctactaggacca

SEQ ID NO: 234 - DNA, 14 bp, synthetic construct
  source 1..14: mol_type = other DNA
ttcctactaggacc

SEQ ID NO: 235 - DNA, 14 bp, synthetic construct
  source 1..14: mol_type = other DNA
tttcctactaggac

SEQ ID NO: 236 - DNA, 14 bp, synthetic construct
  source 1..14: mol_type = other DNA
atttcctactagga

SEQ ID NO: 237 - DNA, 14 bp, synthetic construct
  source 1..14: mol_type = other DNA
tatttcctactagg

SEQ ID NO: 238 - DNA, 14 bp, synthetic construct
  source 1..14: mol_type = other DNA
ttatttcctactag

SEQ ID NO: 239 - DNA, 14 bp, synthetic construct
  source 1..14: mol_type = other DNA
tttatttcctacta

SEQ ID NO: 240 - DNA, 14 bp, synthetic construct
  source 1..14: mol_type = other DNA
atttatttcctact

SEQ ID NO: 241 - DNA, 14 bp, synthetic construct
  source 1..14: mol_type = other DNA
catttatttcctac

SEQ ID NO: 242 - DNA, 14 bp, synthetic construct
  source 1..14: mol_type = other DNA
acatttatttccta

SEQ ID NO: 243 - DNA, 14 bp, synthetic construct
  source 1..14: mol_type = other DNA
cacatttatttcct

SEQ ID NO: 244 - DNA, 14 bp, synthetic construct
  source 1..14: mol_type = other DNA
tcacatttatttcc

SEQ ID NO: 245 - DNA, 14 bp, synthetic construct
  source 1..14: mol_type = other DNA
atcacatttatttc

SEQ ID NO: 246 - DNA, 14 bp, synthetic construct
  source 1..14: mol_type = other DNA
aatcacatttattt

SEQ ID NO: 247 - DNA, 14 bp, synthetic construct
  source 1..14: mol_type = other DNA
aaatcacatttatt

SEQ ID NO: 248 - DNA, 14 bp, synthetic construct
  source 1..14: mol_type = other DNA
caaatcacatttat

SEQ ID NO: 249 - DNA, 14 bp, synthetic construct
  source 1..14: mol_type = other DNA
gcaaatcacattta

SEQ ID NO: 250 - DNA, 14 bp, synthetic construct
  source 1..14: mol_type = other DNA
ggcaaatcacattt

SEQ ID NO: 251 - DNA, 14 bp, synthetic construct
  source 1..14: mol_type = other DNA
aggcaaatcacatt

SEQ ID NO: 252 - DNA, 14 bp, synthetic construct
  source 1..14: mol_type = other DNA
aaggcaaatcacat

SEQ ID NO: 253 - DNA, 14 bp, synthetic construct
  source 1..14: mol_type = other DNA
gaaggcaaatcaca

SEQ ID NO: 254 - DNA, 14 bp, synthetic construct
  source 1..14: mol_type = other DNA
agaaggcaaatcac

SEQ ID NO: 255 - DNA, 14 bp, synthetic construct
  source 1..14: mol_type = other DNA
tagaaggcaaatca

SEQ ID NO: 256 - DNA, 14 bp, synthetic construct
  source 1..14: mol_type = other DNA
ctagaaggcaaatc

SEQ ID NO: 257 - DNA, 14 bp, synthetic construct
  source 1..14: mol_type = other DNA
tctagaaggcaaat

SEQ ID NO: 258 - DNA, 14 bp, synthetic construct
  source 1..14: mol_type = other DNA
ttctagaaggcaaa

SEQ ID NO: 259 - DNA, 14 bp, synthetic construct
  source 1..14: mol_type = other DNA
gttctagaaggcaa

SEQ ID NO: 260 - DNA, 14 bp, synthetic construct
  source 1..14: mol_type = other DNA
tgttctagaaggca

SEQ ID NO: 261 - DNA, 14 bp, synthetic construct
  source 1..14: mol_type = other DNA
ctgttctagaaggc

SEQ ID NO: 262 - DNA, 14 bp, synthetic construct
  source 1..14: mol_type = other DNA
actgttctagaagg

SEQ ID NO: 263 - DNA, 14 bp, synthetic construct
  source 1..14: mol_type = other DNA
tactgttctagaag

SEQ ID NO: 264 - DNA, 14 bp, synthetic construct
  source 1..14: mol_type = other DNA
ctactgttctagaa

SEQ ID NO: 265 - DNA, 14 bp, synthetic construct
  source 1..14: mol_type = other DNA
tctactgttctaga

SEQ ID NO: 266 - DNA, 14 bp, synthetic construct
  source 1..14: mol_type = other DNA
gtctactgttctag

SEQ ID NO: 267 - DNA, 14 bp, synthetic construct
  source 1..14: mol_type = other DNA
tgtctactgttcta

SEQ ID NO: 268 - DNA, 14 bp, synthetic construct
  source 1..14: mol_type = other DNA
gtgtctactgttct

SEQ ID NO: 269 - DNA, 14 bp, synthetic construct
  source 1..14: mol_type = other DNA
tgtgtctactgttc

SEQ ID NO: 270 - DNA, 14 bp, synthetic construct
  source 1..14: mol_type = other DNA
ttgtgtctactgtt

SEQ ID NO: 271 - DNA, 14 bp, synthetic construct
  source 1..14: mol_type = other DNA
tttgtgtctactgt

SEQ ID NO: 272 - DNA, 14 bp, synthetic construct
  source 1..14: mol_type = other DNA
ttttgtgtctactg

SEQ ID NO: 273 - DNA, 14 bp, synthetic construct
  source 1..14: mol_type = other DNA
gttttgtgtctact

SEQ ID NO: 274 - DNA, 14 bp, synthetic construct
  source 1..14: mol_type = other DNA
tgttttgtgtctac

SEQ ID NO: 275 - DNA, 14 bp, synthetic construct
  source 1..14: mol_type = other DNA
ctgttttgtgtcta

SEQ ID NO: 276 - DNA, 14 bp, synthetic construct
  source 1..14: mol_type = other DNA
cctgttttgtgtct

SEQ ID NO: 277 - DNA, 14 bp, synthetic construct
  source 1..14: mol_type = other DNA
gcctgttttgtgtc

SEQ ID NO: 278 - DNA, 14 bp, synthetic construct
  source 1..14: mol_type = other DNA
agcctgttttgtgt

SEQ ID NO: 279 - DNA, 14 bp, synthetic construct
  source 1..14: mol_type = other DNA
gagcctgttttgtg

SEQ ID NO: 280 - DNA, 14 bp, synthetic construct
  source 1..14: mol_type = other DNA
tgagcctgttttgt

SEQ ID NO: 281 - DNA, 14 bp, synthetic construct
  source 1..14: mol_type = other DNA
ctgagcctgttttg

SEQ ID NO: 282 - DNA, 14 bp, synthetic construct
  source 1..14: mol_type = other DNA
cctgagcctgtttt

SEQ ID NO: 283 - DNA, 14 bp, synthetic construct
  source 1..14: mol_type = other DNA
tcctgagcctgttt

SEQ ID NO: 284 - DNA, 14 bp, synthetic construct
  source 1..14: mol_type = other DNA
gtcctgagcctgtt

SEQ ID NO: 285 - DNA, 14 bp, synthetic construct
  source 1..14: mol_type = other DNA
agtcctgagcctgt

SEQ ID NO: 286 - DNA, 14 bp, synthetic construct
  source 1..14: mol_type = other DNA
aagtcctgagcctg

SEQ ID NO: 287 - DNA, 14 bp, synthetic construct
  source 1..14: mol_type = other DNA
taagtcctgagcct

SEQ ID NO: 288 - DNA, 14 bp, synthetic construct
  source 1..14: mol_type = other DNA
ctaagtcctgagcc

SEQ ID NO: 289 - DNA, 14 bp, synthetic construct
  source 1..14: mol_type = other DNA
gctaagtcctgagc

SEQ ID NO: 290 - DNA, 14 bp, synthetic construct
  source 1..14: mol_type = other DNA
tgctaagtcctgag

SEQ ID NO: 291 - DNA, 14 bp, synthetic construct
  source 1..14: mol_type = other DNA
ttgctaagtcctga

SEQ ID NO: 292 - DNA, 14 bp, synthetic construct
  source 1..14: mol_type = other DNA
cttgctaagtcctg

SEQ ID NO: 293 - DNA, 14 bp, synthetic construct
  source 1..14: mol_type = other DNA
tcttgctaagtcct

SEQ ID NO: 294 - DNA, 14 bp, synthetic construct
  source 1..14: mol_type = other DNA
ttcttgctaagtcc

SEQ ID NO: 295 - DNA, 14 bp, synthetic construct
  source 1..14: mol_type = other DNA
cttcttgctaagtc

SEQ ID NO: 296 - DNA, 14 bp, synthetic construct
  source 1..14: mol_type = other DNA
acttcttgctaagt

SEQ ID NO: 297 - DNA, 14 bp, synthetic construct
  source 1..14: mol_type = other DNA
aacttcttgctaag

SEQ ID NO: 298 - DNA, 14 bp, synthetic construct
  source 1..14: mol_type = other DNA
taacttcttgctaa

SEQ ID NO: 299 - DNA, 14 bp, synthetic construct
  source 1..14: mol_type = other DNA
ataacttcttgcta

SEQ ID NO: 300 - DNA, 14 bp, synthetic construct
  source 1..14: mol_type = other DNA
cataacttcttgct

SEQ ID NO: 301 - DNA, 14 bp, synthetic construct
  source 1..14: mol_type = other DNA
ccataacttcttgc

SEQ ID NO: 302 - DNA, 14 bp, synthetic construct
  source 1..14: mol_type = other DNA
tccataacttcttg

SEQ ID NO: 303 - DNA, 14 bp, synthetic construct
  source 1..14: mol_type = other DNA
ttccataacttctt

SEQ ID NO: 304 - DNA, 14 bp, synthetic construct
  source 1..14: mol_type = other DNA
attccataacttct

SEQ ID NO: 305 - DNA, 14 bp, synthetic construct
  source 1..14: mol_type = other DNA
aattccataacttc

SEQ ID NO: 306 - DNA, 14 bp, synthetic construct
  source 1..14: mol_type = other DNA
gaattccataactt

SEQ ID NO: 307 - DNA, 14 bp, synthetic construct
  source 1..14: mol_type = other DNA
ggaattccataact

SEQ ID NO: 308 - DNA, 14 bp, synthetic construct
  source 1..14: mol_type = other DNA
aggaattccataac

SEQ ID NO: 309 - DNA, 14 bp, synthetic construct
  source 1..14: mol_type = other DNA
aaggaattccataa

SEQ ID NO: 310 - DNA, 14 bp, synthetic construct
  source 1..14: mol_type = other DNA
aaaggaattccata

SEQ ID NO: 311 - DNA, 14 bp, synthetic construct
  source 1..14: mol_type = other DNA
aaaaggaattccat

SEQ ID NO: 312 - DNA, 14 bp, synthetic construct
  source 1..14: mol_type = other DNA
taaaaggaattcca

SEQ ID NO: 313 - DNA, 14 bp, synthetic construct
  source 1..14: mol_type = other DNA
ataaaaggaattcc

SEQ ID NO: 314 - DNA, 14 bp, synthetic construct
  source 1..14: mol_type = other DNA
aataaaaggaattc

SEQ ID NO: 315 - DNA, 14 bp, synthetic construct
  source 1..14: mol_type = other DNA
caataaaaggaatt

SEQ ID NO: 316 - DNA, 14 bp, synthetic construct
  source 1..14: mol_type = other DNA
tcaataaaaggaat

SEQ ID NO: 317 - DNA, 14 bp, synthetic construct
  source 1..14: mol_type = other DNA
ttcaataaaaggaa

SEQ ID NO: 318 - DNA, 14 bp, synthetic construct
  source 1..14: mol_type = other DNA
tttcaataaaagga

SEQ ID NO: 319 - DNA, 14 bp, synthetic construct
  source 1..14: mol_type = other DNA
gtttcaataaaagg

SEQ ID NO: 320 - DNA, 14 bp, synthetic construct
  source 1..14: mol_type = other DNA
tgtttcaataaaag

SEQ ID NO: 321 - DNA, 14 bp, synthetic construct
  source 1..14: mol_type = other DNA
atgtttcaataaaa

SEQ ID NO: 322 - DNA, 14 bp, synthetic construct
  source 1..14: mol_type = other DNA
gatgtttcaataaa

SEQ ID NO: 323 - DNA, 14 bp, synthetic construct
  source 1..14: mol_type = other DNA
tgatgtttcaataa

SEQ ID NO: 324 - DNA, 14 bp, synthetic construct
  source 1..14: mol_type = other DNA
ctgatgtttcaata

SEQ ID NO: 325 - DNA, 14 bp, synthetic construct
  source 1..14: mol_type = other DNA
gctgatgtttcaat

SEQ ID NO: 326 - DNA, 14 bp, synthetic construct
  source 1..14: mol_type = other DNA
tgctgatgtttcaa

SEQ ID NO: 327 - DNA, 14 bp, synthetic construct
  source 1..14: mol_type = other DNA
ttgctgatgtttca

SEQ ID NO: 328 - DNA, 14 bp, synthetic construct
  source 1..14: mol_type = other DNA
tttgctgatgtttc

SEQ ID NO: 329 - DNA, 14 bp, synthetic construct
  source 1..14: mol_type = other DNA
ctttgctgatgttt

SEQ ID NO: 330 - DNA, 14 bp, synthetic construct
  source 1..14: mol_type = other DNA
tctttgctgatgtt

SEQ ID NO: 331 - DNA, 14 bp, synthetic construct
  source 1..14: mol_type = other DNA
gtctttgctgatgt

SEQ ID NO: 332 - DNA, 14 bp, synthetic construct
  source 1..14: mol_type = other DNA
tgtctttgctgatg

SEQ ID NO: 333 - DNA, 14 bp, synthetic construct
  source 1..14: mol_type = other DNA
ttgtctttgctgat

SEQ ID NO: 334 - DNA, 14 bp, synthetic construct
  source 1..14: mol_type = other DNA
cttgtctttgctga

SEQ ID NO: 335 - DNA, 14 bp, synthetic construct
  source 1..14: mol_type = other DNA
tcttgtctttgctg

SEQ ID NO: 336 - DNA, 14 bp, synthetic construct
  source 1..14: mol_type = other DNA
gtcttgtctttgct

SEQ ID NO: 337 - DNA, 14 bp, synthetic construct
  source 1..14: mol_type = other DNA
tgtcttgtctttgc

SEQ ID NO: 338 - DNA, 14 bp, synthetic construct
  source 1..14: mol_type = other DNA
ctgtcttgtctttg

SEQ ID NO: 339 - DNA, 14 bp, synthetic construct
  source 1..14: mol_type = other DNA
tctgtcttgtcttt

SEQ ID NO: 340 - DNA, 14 bp, synthetic construct
  source 1..14: mol_type = other DNA
ctctgtcttgtctt

SEQ ID NO: 341 - DNA, 14 bp, synthetic construct
  source 1..14: mol_type = other DNA
tctctgtcttgtct

SEQ ID NO: 342 - DNA, 14 bp, synthetic construct
  source 1..14: mol_type = other DNA
ctctctgtcttgtc

SEQ ID NO: 343 - DNA, 14 bp, synthetic construct
  source 1..14: mol_type = other DNA
actctctgtcttgt

SEQ ID NO: 344 - DNA, 14 bp, synthetic construct
  source 1..14: mol_type = other DNA
cactctctgtcttg

SEQ ID NO: 345 - DNA, 14 bp, synthetic construct
  source 1..14: mol_type = other DNA
ccactctctgtctt

SEQ ID NO: 346 - DNA, 14 bp, synthetic construct
  source 1..14: mol_type = other DNA
tccactctctgtct

SEQ ID NO: 347 - DNA, 14 bp, synthetic construct
  source 1..14: mol_type = other DNA
ctccactctctgtc

SEQ ID NO: 348 - DNA, 14 bp, synthetic construct
  source 1..14: mol_type = other DNA
cctccactctctgt

SEQ ID NO: 349 - DNA, 14 bp, synthetic construct
  source 1..14: mol_type = other DNA
tcctccactctctg

SEQ ID NO: 350 - DNA, 14 bp, synthetic construct
  source 1..14: mol_type = other DNA
atcctccactctct

SEQ ID NO: 351 - DNA, 14 bp, synthetic construct
  source 1..14: mol_type = other DNA
catcctccactctc

SEQ ID NO: 352 - DNA, 14 bp, synthetic construct
  source 1..14: mol_type = other DNA
gcatcctccactct

SEQ ID NO: 353 - DNA, 14 bp, synthetic construct
  source 1..14: mol_type = other DNA
agcatcctccactc

SEQ ID NO: 354 - DNA, 14 bp, synthetic construct
  source 1..14: mol_type = other DNA
aagcatcctccact

SEQ ID NO: 355 - DNA, 14 bp, synthetic construct
  source 1..14: mol_type = other DNA
aaagcatcctccac

SEQ ID NO: 356 - DNA, 14 bp, synthetic construct
  source 1..14: mol_type = other DNA
aaaagcatcctcca

SEQ ID NO: 357 - DNA, 14 bp, synthetic construct
  source 1..14: mol_type = other DNA
aaaaagcatcctcc

SEQ ID NO: 358 - DNA, 14 bp, synthetic construct
  source 1..14: mol_type = other DNA
taaaaagcatcctc

SEQ ID NO: 359 - DNA, 14 bp, synthetic construct
  source 1..14: mol_type = other DNA
ataaaaagcatcct

SEQ ID NO: 360 - DNA, 14 bp, synthetic construct
  source 1..14: mol_type = other DNA
tataaaaagcatcc

SEQ ID NO: 361 - DNA, 14 bp, synthetic construct
  source 1..14: mol_type = other DNA
gtataaaaagcatc

SEQ ID NO: 362 - DNA, 14 bp, synthetic construct
  source 1..14: mol_type = other DNA
tgtataaaaagcat

SEQ ID NO: 363 - DNA, 14 bp, synthetic construct
  source 1..14: mol_type = other DNA
atgtataaaaagca

SEQ ID NO: 364 - DNA, 14 bp, synthetic construct
  source 1..14: mol_type = other DNA
aatgtataaaaagc

SEQ ID NO: 365 - DNA, 14 bp, synthetic construct
  source 1..14: mol_type = other DNA
caatgtataaaaag

SEQ ID NO: 366 - DNA, 14 bp, synthetic construct
  source 1..14: mol_type = other DNA
ccaatgtataaaaa

SEQ ID NO: 367 - DNA, 14 bp, synthetic construct
  source 1..14: mol_type = other DNA
accaatgtataaaa

SEQ ID NO: 368 - DNA, 14 bp, synthetic construct
  source 1..14: mol_type = other DNA
caccaatgtataaa

SEQ ID NO: 369 - DNA, 14 bp, synthetic construct
  source 1..14: mol_type = other DNA
tcaccaatgtataa

SEQ ID NO: 370 - DNA, 14 bp, synthetic construct
  source 1..14: mol_type = other DNA
ctcaccaatgtata

SEQ ID NO: 371 - DNA, 14 bp, synthetic construct
  source 1..14: mol_type = other DNA
tctcaccaatgtat

SEQ ID NO: 372 - DNA, 14 bp, synthetic construct
  source 1..14: mol_type = other DNA
ctctcaccaatgta

SEQ ID NO: 373 - DNA, 14 bp, synthetic construct
  source 1..14: mol_type = other DNA
tctctcaccaatgt

SEQ ID NO: 374 - DNA, 14 bp, synthetic construct
  source 1..14: mol_type = other DNA
ctctctcaccaatg

SEQ ID NO: 375 - DNA, 14 bp, synthetic construct
  source 1..14: mol_type = other DNA
tctctctcaccaat

SEQ ID NO: 376 - DNA, 14 bp, synthetic construct
  source 1..14: mol_type = other DNA
atctctctcaccaa

SEQ ID NO: 377 - DNA, 14 bp, synthetic construct
  source 1..14: mol_type = other DNA
gatctctctcacca

SEQ ID NO: 378 - DNA, 14 bp, synthetic construct
  source 1..14: mol_type = other DNA
ggatctctctcacc

SEQ ID NO: 379 - DNA, 14 bp, synthetic construct
  source 1..14: mol_type = other DNA
cggatctctctcac

SEQ ID NO: 380 - DNA, 14 bp, synthetic construct
  source 1..14: mol_type = other DNA
tcggatctctctca

SEQ ID NO: 381 - DNA, 14 bp, synthetic construct
  source 1..14: mol_type = other DNA
gtcggatctctctc

SEQ ID NO: 382 - DNA, 14 bp, synthetic construct
  source 1..14: mol_type = other DNA
tgtcggatctctct

SEQ ID NO: 383 - DNA, 14 bp, synthetic construct
  source 1..14: mol_type = other DNA
ttgtcggatctctc

SEQ ID NO: 384 - DNA, 14 bp, synthetic construct
  source 1..14: mol_type = other DNA
attgtcggatctct

SEQ ID NO: 385 - DNA, 14 bp, synthetic construct
  source 1..14: mol_type = other DNA
tattgtcggatctc

SEQ ID NO: 386 - DNA, 14 bp, synthetic construct
  source 1..14: mol_type = other DNA
gtattgtcggatct

SEQ ID NO: 387 - DNA, 14 bp, synthetic construct
  source 1..14: mol_type = other DNA
tgtattgtcggatc

SEQ ID NO: 388 - DNA, 14 bp, synthetic construct
  source 1..14: mol_type = other DNA
ctgtattgtcggat

SEQ ID NO: 389 - DNA, 14 bp, synthetic construct
  source 1..14: mol_type = other DNA
tctgtattgtcgga

SEQ ID NO: 390 - DNA, 14 bp, synthetic construct
  source 1..14: mol_type = other DNA
atctgtattgtcgg

SEQ ID NO: 391 - DNA, 14 bp, synthetic construct
  source 1..14: mol_type = other DNA
aatctgtattgtcg

SEQ ID NO: 392 - DNA, 14 bp, synthetic construct
  source 1..14: mol_type = other DNA
caatctgtattgtc

SEQ ID NO: 393 - DNA, 14 bp, synthetic construct
  source 1..14: mol_type = other DNA
tcaatctgtattgt

SEQ ID NO: 394 - DNA, 14 bp, synthetic construct
  source 1..14: mol_type = other DNA
ttcaatctgtattg

SEQ ID NO: 395 - DNA, 14 bp, synthetic construct
  source 1..14: mol_type = other DNA
tttcaatctgtatt

SEQ ID NO: 396 - DNA, 14 bp, synthetic construct
  source 1..14: mol_type = other DNA
ttttcaatctgtat

SEQ ID NO: 397 - DNA, 14 bp, synthetic construct
  source 1..14: mol_type = other DNA
tttttcaatctgta

SEQ ID NO: 398 - DNA, 14 bp, synthetic construct
  source 1..14: mol_type = other DNA
ttttttcaatctgt

SEQ ID NO: 399 - DNA, 14 bp, synthetic construct
  source 1..14: mol_type = other DNA
tttttttcaatctg

SEQ ID NO: 400 - DNA, 14 bp, synthetic construct
  source 1..14: mol_type = other DNA
atttttttcaatct

SEQ ID NO: 401 - DNA, 14 bp, synthetic construct
  source 1..14: mol_type = other DNA
gatttttttcaatc

SEQ ID NO: 402 - DNA, 14 bp, synthetic construct
  source 1..14: mol_type = other DNA
tgatttttttcaat

SEQ ID NO: 403 - DNA, 14 bp, synthetic construct
  source 1..14: mol_type = other DNA
ctgatttttttcaa

SEQ ID NO: 404 - DNA, 14 bp, synthetic construct
  source 1..14: mol_type = other DNA
gctgatttttttca

SEQ ID NO: 405 - DNA, 14 bp, synthetic construct
  source 1..14: mol_type = other DNA
tgctgatttttttc

SEQ ID NO: 406 - DNA, 14 bp, synthetic construct
  source 1..14: mol_type = other DNA
ttgctgattttttt

SEQ ID NO: 407 - DNA, 14 bp, synthetic construct
  source 1..14: mol_type = other DNA
tttgctgatttttt

SEQ ID NO: 408 - DNA, 14 bp, synthetic construct
  source 1..14: mol_type = other DNA
ctttgctgattttt

SEQ ID NO: 409 - DNA, 14 bp, synthetic construct
  source 1..14: mol_type = other DNA
tctttgctgatttt

SEQ ID NO: 410 - DNA, 14 bp, synthetic construct
  source 1..14: mol_type = other DNA
ttctttgctgattt

SEQ ID NO: 411 - DNA, 14 bp, synthetic construct
  source 1..14: mol_type = other DNA
cttctttgctgatt

SEQ ID NO: 412 - DNA, 14 bp, synthetic construct
  source 1..14: mol_type = other DNA
tcttctttgctgat

SEQ ID NO: 413 - DNA, 14 bp, synthetic construct
  source 1..14: mol_type = other DNA
ttcttctttgctga

SEQ ID NO: 414 - DNA, 14 bp, synthetic construct
  source 1..14: mol_type = other DNA
tttcttctttgctg

SEQ ID NO: 415 - DNA, 14 bp, synthetic construct
  source 1..14: mol_type = other DNA
ttttcttctttgct

SEQ ID NO: 416 - DNA, 14 bp, synthetic construct
  source 1..14: mol_type = other DNA
cttttcttctttgc

SEQ ID NO: 417 - DNA, 14 bp, synthetic construct
  source 1..14: mol_type = other DNA
tcttttcttctttg

SEQ ID NO: 418 - DNA, 14 bp, synthetic construct
  source 1..14: mol_type = other DNA
gtcttttcttcttt

SEQ ID NO: 419 - DNA, 14 bp, synthetic construct
  source 1..14: mol_type = other DNA
agtcttttcttctt

SEQ ID NO: 420 - DNA, 14 bp, synthetic construct
  source 1..14: mol_type = other DNA
gagtcttttcttct

SEQ ID NO: 421 - DNA, 14 bp, synthetic construct
  source 1..14: mol_type = other DNA
ggagtcttttcttc

SEQ ID NO: 422 - DNA, 14 bp, synthetic construct
  source 1..14: mol_type = other DNA
aggagtcttttctt

SEQ ID NO: 423 - DNA, 14 bp, synthetic construct
  source 1..14: mol_type = other DNA
caggagtcttttct

SEQ ID NO: 424 - DNA, 14 bp, synthetic construct
  source 1..14: mol_type = other DNA
ccaggagtcttttc

SEQ ID NO: 425 - DNA, 14 bp, synthetic construct
  source 1..14: mol_type = other DNA
gccaggagtctttt

SEQ ID NO: 426 - DNA, 14 bp, synthetic construct
  source 1..14: mol_type = other DNA
agccaggagtcttt

SEQ ID NO: 427 - DNA, 14 bp, synthetic construct
  source 1..14: mol_type = other DNA
cagccaggagtctt

SEQ ID NO: 428 - DNA, 14 bp, synthetic construct
  source 1..14: mol_type = other DNA
acagccaggagtct

SEQ ID NO: 429 - DNA, 14 bp, synthetic construct
  source 1..14: mol_type = other DNA
cacagccaggagtc

SEQ ID NO: 430 - DNA, 14 bp, synthetic construct
  source 1..14: mol_type = other DNA
acacagccaggagt

SEQ ID NO: 431 - DNA, 14 bp, synthetic construct
  source 1..14: mol_type = other DNA
cacacagccaggag

SEQ ID NO: 432 - DNA, 14 bp, synthetic construct
  source 1..14: mol_type = other DNA
tcacacagccagga

SEQ ID NO: 433 - DNA, 14 bp, synthetic construct
  source 1..14: mol_type = other DNA
ttcacacagccagg

SEQ ID NO: 434 - DNA, 14 bp, synthetic construct
  source 1..14: mol_type = other DNA
tttcacacagccag

SEQ ID NO: 435 - DNA, 14 bp, synthetic construct
  source 1..14: mol_type = other DNA
ttttcacacagcca

SEQ ID NO: 436 - DNA, 14 bp, synthetic construct
  source 1..14: mol_type = other DNA
attttcacacagcc

SEQ ID NO: 437 - DNA, 14 bp, synthetic construct
  source 1..14: mol_type = other DNA
aattttcacacagc

SEQ ID NO: 438 - DNA, 14 bp, synthetic construct
  source 1..14: mol_type = other DNA
taattttcacacag

SEQ ID NO: 439 - DNA, 14 bp, synthetic construct
  source 1..14: mol_type = other DNA
ttaattttcacaca

SEQ ID NO: 440 - DNA, 14 bp, synthetic construct
  source 1..14: mol_type = other DNA
tttaattttcacac

SEQ ID NO: 441 - DNA, 14 bp, synthetic construct
  source 1..14: mol_type = other DNA
ttttaattttcaca

SEQ ID NO: 442 - DNA, 14 bp, synthetic construct
  source 1..14: mol_type = other DNA
tttttaattttcac

SEQ ID NO: 443 - DNA, 14 bp, synthetic construct
  source 1..14: mol_type = other DNA
ttttttaattttca

SEQ ID NO: 444 - DNA, 14 bp, synthetic construct
  source 1..14: mol_type = other DNA
attttttaattttc

SEQ ID NO: 445 - DNA, 14 bp, synthetic construct
  source 1..14: mol_type = other DNA
cattttttaatttt

SEQ ID NO: 446 - DNA, 14 bp, synthetic construct
  source 1..14: mol_type = other DNA
gcattttttaattt

SEQ ID NO: 447 - DNA, 14 bp, synthetic construct
  source 1..14: mol_type = other DNA
tgcattttttaatt

SEQ ID NO: 448 - DNA, 14 bp, synthetic construct
  source 1..14: mol_type = other DNA
atgcattttttaat

SEQ ID NO: 449 - DNA, 14 bp, synthetic construct
  source 1..14: mol_type = other DNA
aatgcattttttaa

SEQ ID NO: 450 - DNA, 14 bp, synthetic construct
  source 1..14: mol_type = other DNA
taatgcatttttta

SEQ ID NO: 451 - DNA, 14 bp, synthetic construct
  source 1..14: mol_type = other DNA
ataatgcatttttt

SEQ ID NO: 452 - DNA, 14 bp, synthetic construct
  source 1..14: mol_type = other DNA
tataatgcattttt

SEQ ID NO: 453 - DNA, 14 bp, synthetic construct
  source 1..14: mol_type = other DNA
ttataatgcatttt

SEQ ID NO: 454 - DNA, 14 bp, synthetic construct
  source 1..14: mol_type = other DNA
attataatgcattt

SEQ ID NO: 455 - DNA, 14 bp, synthetic construct
  source 1..14: mol_type = other DNA
cattataatgcatt

SEQ ID NO: 456 - DNA, 14 bp, synthetic construct
  source 1..14: mol_type = other DNA
acattataatgcat

SEQ ID NO: 457 - DNA, 14 bp, synthetic construct
  source 1..14: mol_type = other DNA
tacattataatgca

SEQ ID NO: 458 - DNA, 16 bp, synthetic construct
  source 1..16: mol_type = other DNA
tatcgtcaaggcactc

SEQ ID NO: 459 - DNA, 16 bp, synthetic construct
  source 1..16: mol_type = other DNA
cctcattgcactgtac

SEQ ID NO: 460 - DNA, 16 bp, synthetic construct
  source 1..16: mol_type = other DNA
tatccaaactgcccta

SEQ ID NO: 461 - DNA, 16 bp, synthetic construct
  source 1..16: mol_type = other DNA
tgcctacgccacaagc

SEQ ID NO: 462 - DNA, 16 bp, synthetic construct
  source 1..16: mol_type = other DNA
gcctacgccacaagct

SEQ ID NO: 463 - DNA, 16 bp, synthetic construct
  source 1..16: mol_type = other DNA
cctacgccacaagctc

SEQ ID NO: 464 - DNA, 16 bp, synthetic construct
  source 1..16: mol_type = other DNA
ctacgccacaagctcc

SEQ ID NO: 465 - DNA, 16 bp, synthetic construct
  source 1..16: mol_type = other DNA
tacgccacaagctcca

SEQ ID NO: 466 - DNA, 16 bp, synthetic construct
  source 1..16: mol_type = other DNA
acgccacaagctccaa

SEQ ID NO: 467 - DNA, 16 bp, synthetic construct
  source 1..16: mol_type = other DNA
cgccacaagctccaac

SEQ ID NO: 468 - DNA, 16 bp, synthetic construct
  source 1..16: mol_type = other DNA
gccacaagctccaact

SEQ ID NO: 469 - DNA, 16 bp, synthetic construct
  source 1..16: mol_type = other DNA
ccacaagctccaacta

SEQ ID NO: 470 - DNA, 16 bp, synthetic construct
  source 1..16: mol_type = other DNA
cacaagctccaactac

SEQ ID NO: 471 - DNA, 16 bp, synthetic construct
  source 1..16: mol_type = other DNA
ttgcctacgccatcag

SEQ ID NO: 472 - DNA, 16 bp, synthetic construct
  source 1..16: mol_type = other DNA
tgcctacgccatcagc

SEQ ID NO: 473 - DNA, 16 bp, synthetic construct
  source 1..16: mol_type = other DNA
gcctacgccatcagct

SEQ ID NO: 474 - DNA, 16 bp, synthetic construct
  source 1..16: mol_type = other DNA
cctacgccatcagctc

SEQ ID NO: 475 - DNA, 16 bp, synthetic construct
  source 1..16: mol_type = other DNA
ctacgccatcagctcc

SEQ ID NO: 476 - DNA, 16 bp, synthetic construct
  source 1..16: mol_type = other DNA
tacgccatcagctcca

SEQ ID NO: 477 - DNA, 16 bp, synthetic construct
  source 1..16: mol_type = other DNA
acgccatcagctccaa

SEQ ID NO: 478 - DNA, 16 bp, synthetic construct
  source 1..16: mol_type = other DNA
cgccatcagctccaac

SEQ ID NO: 479 - DNA, 16 bp, synthetic construct
  source 1..16: mol_type = other DNA
gccatcagctccaact

SEQ ID NO: 480 - DNA, 16 bp, synthetic construct
  source 1..16: mol_type = other DNA
ccatcagctccaacta

SEQ ID NO: 481 - DNA, 15 bp, synthetic construct
  source 1..15: mol_type = other DNA
acgccacaagctcca

SEQ ID NO: 482 - DNA, 14 bp, synthetic construct
  source 1..14: mol_type = other DNA
cgccacaagctcca

SEQ ID NO: 483 - DNA, 14 bp, synthetic construct
  source 1..14: mol_type = other DNA
acgccacaagctcc

SEQ ID NO: 484 - DNA, 15 bp, synthetic construct
  source 1..15: mol_type = other DNA
ctacgccatcagctc

SEQ ID NO: 485 - DNA, 14 bp, synthetic construct
  source 1..14: mol_type = other DNA
ctacgccatcagct

SEQ ID NO: 486 - DNA, 14 bp, synthetic construct
  source 1..14: mol_type = other DNA
tacgccatcagctc

SEQ ID NO: 487 - DNA, 14 bp, synthetic construct
  source 1..14: mol_type = other DNA
acgccatcagctcc

SEQ ID NO: 488 - DNA, 16 bp, synthetic construct
  source 1..16: mol_type = other DNA
cctacgccagcagctc

SEQ ID NO: 489 - DNA, 15 bp, synthetic construct
  source 1..15: mol_type = other DNA
ctacgccagcagctc

SEQ ID NO: 490 - DNA, 14 bp, synthetic construct
  source 1..14: mol_type = other DNA
ctacgccagcagct

SEQ ID NO: 491 - DNA, 14 bp, synthetic construct
  source 1..14: mol_type = other DNA
tacgccagcagctc

SEQ ID NO: 492 - DNA, 14 bp, synthetic construct
  source 1..14: mol_type = other DNA
acgccagcagctcc

SEQ ID NO: 493 - DNA, 16 bp, synthetic construct
  source 1..16: mol_type = other DNA
cctacgccaacagctc

SEQ ID NO: 494 - DNA, 15 bp, synthetic construct
  source 1..15: mol_type = other DNA
ctacgccaacagctc

SEQ ID NO: 495 - DNA, 14 bp, synthetic construct
  source 1..14: mol_type = other DNA
ctacgccaacagct

SEQ ID NO: 496 - DNA, 14 bp, synthetic construct
  source 1..14: mol_type = other DNA
tacgccaacagctc

SEQ ID NO: 497 - DNA, 14 bp, synthetic construct
  source 1..14: mol_type = other DNA
acgccaacagctcc

SEQ ID NO: 498 - DNA, 14 bp, synthetic construct
  source 1..14: mol_type = other DNA
cgccacaagctcca

SEQ ID NO: 499 - DNA, 13 bp, synthetic construct
  source 1..13: mol_type = other DNA
cgccacaagctcc

SEQ ID NO: 500 - DNA, 13 bp, synthetic construct
  source 1..13: mol_type = other DNA
acgccatcagctc

SEQ ID NO: 501 - DNA, 13 bp, synthetic construct
  source 1..13: mol_type = other DNA
acgccaacagctc

SEQ ID NO: 502 - DNA, 14 bp, synthetic construct
  source 1..14: mol_type = other DNA
acgccaacagctcc

SEQ ID NO: 503 - DNA, 16 bp, synthetic construct
  source 1..16: mol_type = other DNA
ctacgccagcagctcc

SEQ ID NO: 504 - DNA, 13 bp, synthetic construct
  source 1..13: mol_type = other DNA
acgccagcagctc

SEQ ID NO: 505 - DNA, 14 bp, synthetic construct
  source 1..14: mol_type = other DNA
cctacgcaaccacg

SEQ ID NO: 506 - DNA, 14 bp, synthetic construct
  source 1..14: mol_type = other DNA
ctacgcaaccacgt

SEQ ID NO: 507 - DNA, 13 bp, synthetic construct
  source 1..13: mol_type = other DNA
ctacgcaaccacg

SEQ ID NO: 508 - DNA, 14 bp, synthetic construct
  source 1..14: mol_type = other DNA
gcctacgtaaccac

SEQ ID NO: 509 - DNA, 14 bp, synthetic construct
  source 1..14: mol_type = other DNA
cctacgtaaccacg

SEQ ID NO: 510 - DNA, 13 bp, synthetic construct
  source 1..13: mol_type = other DNA
cctacgtaaccac

SEQ ID NO: 511 - DNA, 13 bp, synthetic construct
  source 1..13: mol_type = other DNA
acgccaacagctc

SEQ ID NO: 512 - DNA, 14 bp, synthetic construct
  source 1..14: mol_type = other DNA
acgccaacagctcc

SEQ ID NO: 513 - DNA, 14 bp, synthetic construct
  source 1..14: mol_type = other DNA
acgccaacagctcc

SEQ ID NO: 514 - DNA, 13 bp, synthetic construct
  source 1..13: mol_type = other DNA
acgccaacagctc

SEQ ID NO: 515 - DNA, 13 bp, synthetic construct
  source 1..13: mol_type = other DNA
acgccaacagctc

SEQ ID NO: 516 - DNA, 13 bp, synthetic construct
  source 1..13: mol_type = other DNA
acgccaacagctc

SEQ ID NO: 517 - DNA, 14 bp, synthetic construct
  source 1..14: mol_type = other DNA
tacgccaacagctc

SEQ ID NO: 518 - DNA, 14 bp, synthetic construct
  source 1..14: mol_type = other DNA
tacgccaacagctc

SEQ ID NO: 519 - DNA, 14 bp, synthetic construct
  source 1..14: mol_type = other DNA
tacgccaacagctc

SEQ ID NO: 520 - DNA, 14 bp, synthetic construct
  source 1..14: mol_type = other DNA
tacgccaacagctc

SEQ ID NO: 521 - DNA, 15 bp, synthetic construct
  source 1..15: mol_type = other DNA
tadcgccaacagctc

SEQ ID NO: 522 - DNA, 14 bp, synthetic construct
  source 1..14: mol_type = other DNA
tacgccaacagctc

SEQ ID NO: 523 - DNA, 14 bp, synthetic construct
  source 1..14: mol_type = other DNA
tacgccaacagctc

SEQ ID NO: 524 - DNA, 14 bp, synthetic construct
  source 1..14: mol_type = other DNA
tacgccaacagctc

SEQ ID NO: 525 - DNA, 16 bp, synthetic construct
  source 1..16: mol_type = other DNA
gctattaggagtcttt

SEQ ID NO: 526 - DNA, 14 bp, synthetic construct
  source 1..14: mol_type = other DNA
tacgccacaagctc

SEQ ID NO: 527 - DNA, 13 bp, synthetic construct
  source 1..13: mol_type = other DNA
acgccacaagctc

SEQ ID NO: 528 - DNA, 14 bp, synthetic construct
  source 1..14: mol_type = other DNA
ctacgccacaagct

SEQ ID NO: 529 - DNA, 15 bp, synthetic construct
  source 1..15: mol_type = other DNA
ctacgccacaagctc

SEQ ID NO: 530 - DNA, 14 bp, synthetic construct
  source 1..14: mol_type = other DNA
acgtctatacacca

SEQ ID NO: 531 - DNA, 14 bp, synthetic construct
  source 1..14: mol_type = other DNA
gagctgatggcgta

SEQ ID NO: 532 - DNA, 16 bp, synthetic construct
  source 1..16: mol_type = other DNA
cacgtctatacaccac

SEQ ID NO: 533 - DNA, 16 bp, synthetic construct
  source 1..16: mol_type = other DNA
tttctgaggattatcg

SEQ ID NO: 534 - DNA, 16 bp, synthetic construct
  source 1..16: mol_type = other DNA
gctcgaatgatcggaa

SEQ ID NO: 535 - DNA, 16 bp, synthetic construct
  source 1..16: mol_type = other DNA
attcgactcaccacag

SEQ ID NO: 536 - DNA, 16 bp, synthetic construct
  source 1..16: mol_type = other DNA
tcgcctattgactgca

SEQ ID NO: 537 - DNA, 16 bp, synthetic construct
  source 1..16: mol_type = other DNA
tcgctcctctacatct

SEQ ID NO: 538 - DNA, 16 bp, synthetic construct
  source 1..16: mol_type = other DNA
ttcccatccatcgata

SEQ ID NO: 539 - DNA, 16 bp, synthetic construct
  source 1..16: mol_type = other DNA
cccatcaaatgcacca

SEQ ID NO: 540 - DNA, 14 bp, synthetic construct
  source 1..14: mol_type = other DNA
aggtcgttgttggg

SEQ ID NO: 541 - DNA, 14 bp, synthetic construct
  source 1..14: mol_type = other DNA
gaggtcgttgttgg

SEQ ID NO: 542 - DNA, 14 bp, synthetic construct
  source 1..14: mol_type = other DNA
ccggaggtcgttgt

SEQ ID NO: 543 - DNA, 14 bp, synthetic construct
  source 1..14: mol_type = other DNA
gcggaggtctcgcg

SEQ ID NO: 544 - DNA, 14 bp, synthetic construct
  source 1..14: mol_type = other DNA
agttgggactccga

SEQ ID NO: 545 - DNA, 14 bp, synthetic construct
  source 1..14: mol_type = other DNA
gcgtagttgggact

SEQ ID NO: 546 - DNA, 14 bp, synthetic construct
  source 1..14: mol_type = other DNA
cgcgtagttgggac

SEQ ID NO: 547 - DNA, 14 bp, synthetic construct
  source 1..14: mol_type = other DNA
cggcatcccgcacc

SEQ ID NO: 548 - DNA, 14 bp, synthetic construct
  source 1..14: mol_type = other DNA
ggagaacggacgcg

SEQ ID NO: 549 - DNA, 14 bp, synthetic construct
  source 1..14: mol_type = other DNA
tggagaacggacgc

SEQ ID NO: 550 - DNA, 14 bp, synthetic construct
  source 1..14: mol_type = other DNA
taccagccgtggag

SEQ ID NO: 551 - DNA, 14 bp, synthetic construct
  source 1..14: mol_type = other DNA
gtaccagccgtgga

SEQ ID NO: 552 - DNA, 14 bp, synthetic construct
  source 1..14: mol_type = other DNA
cccgacacaggtac

SEQ ID NO: 553 - DNA, 14 bp, synthetic construct
  source 1..14: mol_type = other DNA
gccgggctagccct

SEQ ID NO: 554 - DNA, 14 bp, synthetic construct
  source 1..14: mol_type = other DNA
agccgggctagccc

SEQ ID NO: 555 - DNA, 14 bp, synthetic construct
  source 1..14: mol_type = other DNA
agcgcggaacaggg

SEQ ID NO: 556 - DNA, 14 bp, synthetic construct
  source 1..14: mol_type = other DNA
cagcgcggaacagg

SEQ ID NO: 557 - DNA, 14 bp, synthetic construct
  source 1..14: mol_type = other DNA
cgcagcgcggaaca

SEQ ID NO: 558 - DNA, 14 bp, synthetic construct
  source 1..14: mol_type = other DNA
tcgcagcgcggaac

SEQ ID NO: 559 - DNA, 14 bp, synthetic construct
  source 1..14: mol_type = other DNA
agggcgagctcgca

SEQ ID NO: 560 - DNA, 14 bp, synthetic construct
  source 1..14: mol_type = other DNA
aaactcgtagggca

SEQ ID NO: 561 - DNA, 14 bp, synthetic construct
  source 1..14: mol_type = other DNA
aaaactcgtagggc

SEQ ID NO: 562 - DNA, 14 bp, synthetic construct
  source 1..14: mol_type = other DNA
gggcgcgttctctt

SEQ ID NO: 563 - DNA, 14 bp, synthetic construct
  source 1..14: mol_type = other DNA
ttgggcgcgttctc

SEQ ID NO: 564 - DNA, 14 bp, synthetic construct
  source 1..14: mol_type = other DNA
acttgggcgcgttc

SEQ ID NO: 565 - DNA, 14 bp, synthetic construct
  source 1..14: mol_type = other DNA
cgccacttgggcgc

SEQ ID NO: 566 - DNA, 14 bp, synthetic construct
  source 1..14: mol_type = other DNA
gtagtccccgccac

SEQ ID NO: 567 - DNA, 14 bp, synthetic construct
  source 1..14: mol_type = other DNA
tcgagagtaggatg

SEQ ID NO: 568 - DNA, 14 bp, synthetic construct
  source 1..14: mol_type = other DNA
ttagactcgagagt

SEQ ID NO: 569 - DNA, 14 bp, synthetic construct
  source 1..14: mol_type = other DNA
tcattagactcgag

SEQ ID NO: 570 - DNA, 14 bp, synthetic construct
  source 1..14: mol_type = other DNA
atcattagactcga

SEQ ID NO: 571 - DNA, 14 bp, synthetic construct
  source 1..14: mol_type = other DNA
ggcatagcatattt

SEQ ID NO: 572 - DNA, 14 bp, synthetic construct
  source 1..14: mol_type = other DNA
gaagcacttcgggg

SEQ ID NO: 573 - DNA, 14 bp, synthetic construct
  source 1..14: mol_type = other DNA
acgttcctctacat

SEQ ID NO: 574 - DNA, 14 bp, synthetic construct
  source 1..14: mol_type = other DNA
tatcaattggatga

SEQ ID NO: 575 - DNA, 14 bp, synthetic construct
  source 1..14: mol_type = other DNA
cccatttatcaatt

SEQ ID NO: 576 - DNA, 14 bp, synthetic construct
  source 1..14: mol_type = other DNA
gcccatttatcaat

SEQ ID NO: 577 - DNA, 14 bp, synthetic construct
  source 1..14: mol_type = other DNA
tgcccatttatcaa

SEQ ID NO: 578 - DNA, 14 bp, synthetic construct
  source 1..14: mol_type = other DNA
aggacctcctttaa

SEQ ID NO: 579 - DNA, 14 bp, synthetic construct
  source 1..14: mol_type = other DNA
taggacctccttta

SEQ ID NO: 580 - DNA, 14 bp, synthetic construct
  source 1..14: mol_type = other DNA
ataacctaggacct

SEQ ID NO: 581 - DNA, 14 bp, synthetic construct
  source 1..14: mol_type = other DNA
gccgtatatttctt

SEQ ID NO: 582 - DNA, 14 bp, synthetic construct
  source 1..14: mol_type = other DNA
tgccgtatatttct

SEQ ID NO: 583 - DNA, 14 bp, synthetic construct
  source 1..14: mol_type = other DNA
tcataatgccgtat

SEQ ID NO: 584 - DNA, 14 bp, synthetic construct
  source 1..14: mol_type = other DNA
atttcataatgccg

SEQ ID NO: 585 - DNA, 14 bp, synthetic construct
  source 1..14: mol_type = other DNA
gctcttcgtcagtt

SEQ ID NO: 586 - DNA, 14 bp, synthetic construct
  source 1..14: mol_type = other DNA
ggaaggctcttcgt

SEQ ID NO: 587 - DNA, 14 bp, synthetic construct
  source 1..14: mol_type = other DNA
tcatagtaagtttg

SEQ ID NO: 588 - DNA, 14 bp, synthetic construct
  source 1..14: mol_type = other DNA
atcatagtaagttt

SEQ ID NO: 589 - DNA, 14 bp, synthetic construct
  source 1..14: mol_type = other DNA
tatatattgtcgaa

SEQ ID NO: 590 - DNA, 14 bp, synthetic construct
  source 1..14: mol_type = other DNA
tagatttagttccc

SEQ ID NO: 591 - DNA, 14 bp, synthetic construct
  source 1..14: mol_type = other DNA
gcgactaaatatat

SEQ ID NO: 592 - DNA, 14 bp, synthetic construct
  source 1..14: mol_type = other DNA
tatctactatgcga

SEQ ID NO: 593 - DNA, 14 bp, synthetic construct
  source 1..14: mol_type = other DNA
atggcccagtaact

SEQ ID NO: 594 - DNA, 14 bp, synthetic construct
  source 1..14: mol_type = other DNA
cttctatatggccc

SEQ ID NO: 595 - DNA, 14 bp, synthetic construct
  source 1..14: mol_type = other DNA
cctactagtggatg

SEQ ID NO: 596 - DNA, 14 bp, synthetic construct
  source 1..14: mol_type = other DNA
cgattcatatggat

SEQ ID NO: 597 - DNA, 14 bp, synthetic construct
  source 1..14: mol_type = other DNA
acgattcatatgga

SEQ ID NO: 598 - DNA, 14 bp, synthetic construct
  source 1..14: mol_type = other DNA
catacgattcatat

SEQ ID NO: 599 - DNA, 14 bp, synthetic construct
  source 1..14: mol_type = other DNA
tcgagcatacgatt

SEQ ID NO: 600 - DNA, 14 bp, synthetic construct
  source 1..14: mol_type = other DNA
atctcgagcatacg

SEQ ID NO: 601 - DNA, 14 bp, synthetic construct
  source 1..14: mol_type = other DNA
atatctcgagcata

SEQ ID NO: 602 - DNA, 14 bp, synthetic construct
  source 1..14: mol_type = other DNA
cgcaaaatatctcg

SEQ ID NO: 603 - DNA, 14 bp, synthetic construct
  source 1..14: mol_type = other DNA
aacgatcatgaaaa

SEQ ID NO: 604 - DNA, 14 bp, synthetic construct
  source 1..14: mol_type = other DNA
actaaggaaacgat

SEQ ID NO: 605 - DNA, 14 bp, synthetic construct
  source 1..14: mol_type = other DNA
gcctattgactgca

SEQ ID NO: 606 - DNA, 14 bp, synthetic construct
  source 1..14: mol_type = other DNA
cgcctattgactgc

SEQ ID NO: 607 - DNA, 14 bp, synthetic construct
  source 1..14: mol_type = other DNA
ccttcgcctattga

SEQ ID NO: 608 - DNA, 14 bp, synthetic construct
  source 1..14: mol_type = other DNA
accttcgcctattg

SEQ ID NO: 609 - DNA, 14 bp, synthetic construct
  source 1..14: mol_type = other DNA
gaaaccttcgccta

SEQ ID NO: 610 - DNA, 14 bp, synthetic construct
  source 1..14: mol_type = other DNA
gcctgggtaaaaca

SEQ ID NO: 611 - DNA, 14 bp, synthetic construct
  source 1..14: mol_type = other DNA
ttcgttttgcaaga

SEQ ID NO: 612 - DNA, 14 bp, synthetic construct
  source 1..14: mol_type = other DNA
cttcgttttgcaag

SEQ ID NO: 613 - DNA, 14 bp, synthetic construct
  source 1..14: mol_type = other DNA
aaccgacatgcaga

SEQ ID NO: 614 - DNA, 14 bp, synthetic construct
  source 1..14: mol_type = other DNA
actataaaaccgac

SEQ ID NO: 615 - DNA, 14 bp, synthetic construct
  source 1..14: mol_type = other DNA
gactataaaaccga

SEQ ID NO: 616 - DNA, 14 bp, synthetic construct
  source 1..14: mol_type = other DNA
ctagttgtttcccc

SEQ ID NO: 617 - DNA, 14 bp, synthetic construct
  source 1..14: mol_type = other DNA
acaacactgtccaa

SEQ ID NO: 618 - DNA, 14 bp, synthetic construct
  source 1..14: mol_type = other DNA
attacaacactgtc

SEQ ID NO: 619 - DNA, 14 bp, synthetic construct
  source 1..14: mol_type = other DNA
cattacaacactgt

SEQ ID NO: 620 - DNA, 14 bp, synthetic construct
  source 1..14: mol_type = other DNA
tcattacaacactg

SEQ ID NO: 621 - DNA, 14 bp, synthetic construct
  source 1..14: mol_type = other DNA
aattcattacaaca

SEQ ID NO: 622 - DNA, 14 bp, synthetic construct
  source 1..14: mol_type = other DNA
tacacgtgtaagag

SEQ ID NO: 623 - DNA, 14 bp, synthetic construct
  source 1..14: mol_type = other DNA
ctacacgtgtaaga

SEQ ID NO: 624 - DNA, 14 bp, synthetic construct
  source 1..14: mol_type = other DNA
taagacgatattct

SEQ ID NO: 625 - DNA, 14 bp, synthetic construct
  source 1..14: mol_type = other DNA
tttaagacgatatt

SEQ ID NO: 626 - DNA, 14 bp, synthetic construct
  source 1..14: mol_type = other DNA
tcgcataaaaaact

SEQ ID NO: 627 - DNA, 14 bp, synthetic construct
  source 1..14: mol_type = other DNA
cctttcgcataaaa

SEQ ID NO: 628 - DNA, 14 bp, synthetic construct
  source 1..14: mol_type = other DNA
tacctttcgcataa

SEQ ID NO: 629 - DNA, 14 bp, synthetic construct
  source 1..14: mol_type = other DNA
tgtacctttcgcat

SEQ ID NO: 630 - DNA, 14 bp, synthetic construct
  source 1..14: mol_type = other DNA
tactccggtactgt

SEQ ID NO: 631 - DNA, 14 bp, synthetic construct
  source 1..14: mol_type = other DNA
acgtaagcctctct

SEQ ID NO: 632 - DNA, 14 bp, synthetic construct
  source 1..14: mol_type = other DNA
tggtacgtaagcct

SEQ ID NO: 633 - DNA, 14 bp, synthetic construct
  source 1..14: mol_type = other DNA
tatggtacgtaagc

SEQ ID NO: 634 - DNA, 14 bp, synthetic construct
  source 1..14: mol_type = other DNA
acatatggtacgta

SEQ ID NO: 635 - DNA, 14 bp, synthetic construct
  source 1..14: mol_type = other DNA
gtacatatggtacg

SEQ ID NO: 636 - DNA, 14 bp, synthetic construct
  source 1..14: mol_type = other DNA
tgcgtacatatggt

SEQ ID NO: 637 - DNA, 14 bp, synthetic construct
  source 1..14: mol_type = other DNA
cgaacaaaattggg

SEQ ID NO: 638 - DNA, 14 bp, synthetic construct
  source 1..14: mol_type = other DNA
ccgaacaaaattgg

SEQ ID NO: 639 - DNA, 14 bp, synthetic construct
  source 1..14: mol_type = other DNA
gaaatgtccgaaca

SEQ ID NO: 640 - DNA, 14 bp, synthetic construct
  source 1..14: mol_type = other DNA
taagaaatgtccga

SEQ ID NO: 641 - DNA, 14 bp, synthetic construct
  source 1..14: mol_type = other DNA
attataagactcag

SEQ ID NO: 642 - DNA, 14 bp, synthetic construct
  source 1..14: mol_type = other DNA
ctatgcgatcagct

SEQ ID NO: 643 - DNA, 14 bp, synthetic construct
  source 1..14: mol_type = other DNA
ctatagctatgcga

SEQ ID NO: 644 - DNA, 14 bp, synthetic construct
  source 1..14: mol_type = other DNA
tgcactcaagggtt

SEQ ID NO: 645 - DNA, 14 bp, synthetic construct
  source 1..14: mol_type = other DNA
tcgcagttgcacag

SEQ ID NO: 646 - DNA, 14 bp, synthetic construct
  source 1..14: mol_type = other DNA
ttaatactcgcagt

SEQ ID NO: 647 - DNA, 14 bp, synthetic construct
  source 1..14: mol_type = other DNA
tttaatactcgcag

SEQ ID NO: 648 - DNA, 14 bp, synthetic construct
  source 1..14: mol_type = other DNA
gtgccgacatacat

SEQ ID NO: 649 - DNA, 14 bp, synthetic construct
  source 1..14: mol_type = other DNA
cagtgccgacatac

SEQ ID NO: 650 - DNA, 14 bp, synthetic construct
  source 1..14: mol_type = other DNA
ttccattcgttgca

SEQ ID NO: 651 - DNA, 14 bp, synthetic construct
  source 1..14: mol_type = other DNA
cttccattcgttgc

SEQ ID NO: 652 - DNA, 14 bp, synthetic construct
  source 1..14: mol_type = other DNA
gatggattcaaccc

SEQ ID NO: 653 - DNA, 14 bp, synthetic construct
  source 1..14: mol_type = other DNA
atgccactcaactg

SEQ ID NO: 654 - DNA, 14 bp, synthetic construct
  source 1..14: mol_type = other DNA
ggtctgcttatatg

SEQ ID NO: 655 - DNA, 14 bp, synthetic construct
  source 1..14: mol_type = other DNA
tcgagcaatttcta

SEQ ID NO: 656 - DNA, 14 bp, synthetic construct
  source 1..14: mol_type = other DNA
aagtgagttgtcga

SEQ ID NO: 657 - DNA, 14 bp, synthetic construct
  source 1..14: mol_type = other DNA
tcggtatagatctg

SEQ ID NO: 658 - DNA, 14 bp, synthetic construct
  source 1..14: mol_type = other DNA
tacagctcggtata

SEQ ID NO: 659 - DNA, 14 bp, synthetic construct
  source 1..14: mol_type = other DNA
ctgtacagctcggt

SEQ ID NO: 660 - DNA, 14 bp, synthetic construct
  source 1..14: mol_type = other DNA
tccaactaattctg

SEQ ID NO: 661 - DNA, 14 bp, synthetic construct
  source 1..14: mol_type = other DNA
agattaggagagtt

SEQ ID NO: 662 - DNA, 14 bp, synthetic construct
  source 1..14: mol_type = other DNA
atgtcgaatcattt

SEQ ID NO: 663 - DNA, 14 bp, synthetic construct
  source 1..14: mol_type = other DNA
ggtggtatgtcgaa

SEQ ID NO: 664 - DNA, 14 bp, synthetic construct
  source 1..14: mol_type = other DNA
tggtggtatgtcga

SEQ ID NO: 665 - DNA, 14 bp, synthetic construct
  source 1..14: mol_type = other DNA
ctcaataattcgac

SEQ ID NO: 666 - DNA, 14 bp, synthetic construct
  source 1..14: mol_type = other DNA
tctcaataattcga

SEQ ID NO: 667 - DNA, 14 bp, synthetic construct
  source 1..14: mol_type = other DNA
tggtctagtctgta

SEQ ID NO: 668 - DNA, 14 bp, synthetic construct
  source 1..14: mol_type = other DNA
ggcgacttggtatt

SEQ ID NO: 669 - DNA, 14 bp, synthetic construct
  source 1..14: mol_type = other DNA
tctggcgacttggt

SEQ ID NO: 670 - DNA, 14 bp, synthetic construct
  source 1..14: mol_type = other DNA
attaatagacctga

SEQ ID NO: 671 - DNA, 14 bp, synthetic construct
  source 1..14: mol_type = other DNA
gattaatagacctg

SEQ ID NO: 672 - DNA, 14 bp, synthetic construct
  source 1..14: mol_type = other DNA
tggattaatagacc

SEQ ID NO: 673 - DNA, 14 bp, synthetic construct
  source 1..14: mol_type = other DNA
cgtaaacagtaagg

SEQ ID NO: 674 - DNA, 14 bp, synthetic construct
  source 1..14: mol_type = other DNA
actcgtaaacagta

SEQ ID NO: 675 - DNA, 14 bp, synthetic construct
  source 1..14: mol_type = other DNA
gattctactcgtaa

SEQ ID NO: 676 - DNA, 14 bp, synthetic construct
  source 1..14: mol_type = other DNA
ctgattctactcgt

SEQ ID NO: 677 - DNA, 14 bp, synthetic construct
  source 1..14: mol_type = other DNA
catcggattcaagt

SEQ ID NO: 678 - DNA, 14 bp, synthetic construct
  source 1..14: mol_type = other DNA
tcccatcggattca

SEQ ID NO: 679 - DNA, 14 bp, synthetic construct
  source 1..14: mol_type = other DNA
ctatttcccatcgg

SEQ ID NO: 680 - DNA, 14 bp, synthetic construct
  source 1..14: mol_type = other DNA
tcgtggttctattt

SEQ ID NO: 681 - DNA, 14 bp, synthetic construct
  source 1..14: mol_type = other DNA
ggtttcgtggttct

SEQ ID NO: 682 - DNA, 14 bp, synthetic construct
  source 1..14: mol_type = other DNA
tagggtttcgtggt

SEQ ID NO: 683 - DNA, 14 bp, synthetic construct
  source 1..14: mol_type = other DNA
cttagggtttcgtg

SEQ ID NO: 684 - DNA, 14 bp, synthetic construct
  source 1..14: mol_type = other DNA
agaggcttagggtt

SEQ ID NO: 685 - DNA, 14 bp, synthetic construct
  source 1..14: mol_type = other DNA
ggggatagctatat

SEQ ID NO: 686 - DNA, 14 bp, synthetic construct
  source 1..14: mol_type = other DNA
atggtacctggtct

SEQ ID NO: 687 - DNA, 14 bp, synthetic construct
  source 1..14: mol_type = other DNA
tcctactataacta

SEQ ID NO: 688 - DNA, 14 bp, synthetic construct
  source 1..14: mol_type = other DNA
ggatcctactataa

SEQ ID NO: 689 - DNA, 14 bp, synthetic construct
  source 1..14: mol_type = other DNA
gttaacggtgttct

SEQ ID NO: 690 - DNA, 14 bp, synthetic construct
  source 1..14: mol_type = other DNA
tgttaacggtgttc

SEQ ID NO: 691 - DNA, 14 bp, synthetic construct
  source 1..14: mol_type = other DNA
ggcggaggtgttaa

SEQ ID NO: 692 - DNA, 14 bp, synthetic construct
  source 1..14: mol_type = other DNA
tgatcggaatcaaa

SEQ ID NO: 693 - DNA, 14 bp, synthetic construct
  source 1..14: mol_type = other DNA
aatgatcggaatca

SEQ ID NO: 694 - DNA, 14 bp, synthetic construct
  source 1..14: mol_type = other DNA
tcgaatgatcggaa

SEQ ID NO: 695 - DNA, 14 bp, synthetic construct
  source 1..14: mol_type = other DNA
gctcgaatgatcgg

SEQ ID NO: 696 - DNA, 14 bp, synthetic construct
  source 1..14: mol_type = other DNA
aaagggctcgaatg

SEQ ID NO: 697 - DNA, 14 bp, synthetic construct
  source 1..14: mol_type = other DNA
gaaaagggctcgaa

SEQ ID NO: 698 - DNA, 14 bp, synthetic construct
  source 1..14: mol_type = other DNA
ttaaacttatagat

SEQ ID NO: 699 - DNA, 14 bp, synthetic construct
  source 1..14: mol_type = other DNA
cagattctggtcgt

SEQ ID NO: 700 - DNA, 14 bp, synthetic construct
  source 1..14: mol_type = other DNA
aatgcttagacata

SEQ ID NO: 701 - DNA, 14 bp, synthetic construct
  source 1..14: mol_type = other DNA
gtgggttgcctagg

SEQ ID NO: 702 - DNA, 14 bp, synthetic construct
  source 1..14: mol_type = other DNA
tcgtggtgaatagg

SEQ ID NO: 703 - DNA, 14 bp, synthetic construct
  source 1..14: mol_type = other DNA
tatcgtggtgaata

SEQ ID NO: 704 - DNA, 14 bp, synthetic construct
  source 1..14: mol_type = other DNA
atattgaatatcgt

SEQ ID NO: 705 - DNA, 14 bp, synthetic construct
  source 1..14: mol_type = other DNA
cggtctgatattga

SEQ ID NO: 706 - DNA, 14 bp, synthetic construct
  source 1..14: mol_type = other DNA
ggtccggtctgata

SEQ ID NO: 707 - DNA, 14 bp, synthetic construct
  source 1..14: mol_type = other DNA
aggtccggtctgat

SEQ ID NO: 708 - DNA, 14 bp, synthetic construct
  source 1..14: mol_type = other DNA
atagaggtccggtc

SEQ ID NO: 709 - DNA, 14 bp, synthetic construct
  source 1..14: mol_type = other DNA
gatagaggtccggt

SEQ ID NO: 710 - DNA, 14 bp, synthetic construct
  source 1..14: mol_type = other DNA
tcgtggtggtaata

SEQ ID NO: 711 - DNA, 14 bp, synthetic construct
  source 1..14: mol_type = other DNA
cacaggttctcgtg

SEQ ID NO: 712 - DNA, 14 bp, synthetic construct
  source 1..14: mol_type = other DNA
aagtgctttgtcgt

SEQ ID NO: 713 - DNA, 14 bp, synthetic construct
  source 1..14: mol_type = other DNA
agtcgttagtgttt

SEQ ID NO: 714 - DNA, 14 bp, synthetic construct
  source 1..14: mol_type = other DNA
gaagagtcgttagt

SEQ ID NO: 715 - DNA, 14 bp, synthetic construct
  source 1..14: mol_type = other DNA
agaagagtcgttag

SEQ ID NO: 716 - DNA, 14 bp, synthetic construct
  source 1..14: mol_type = other DNA
aattacatctaggt

SEQ ID NO: 717 - DNA, 14 bp, synthetic construct
  source 1..14: mol_type = other DNA
gtcggtctttccat

SEQ ID NO: 718 - DNA, 14 bp, synthetic construct
  source 1..14: mol_type = other DNA
taggtcggtctttc

SEQ ID NO: 719 - DNA, 14 bp, synthetic construct
  source 1..14: mol_type = other DNA
acactagactgtgt

SEQ ID NO: 720 - DNA, 14 bp, synthetic construct
  source 1..14: mol_type = other DNA
tgtcatgtgggcct

SEQ ID NO: 721 - DNA, 14 bp, synthetic construct
  source 1..14: mol_type = other DNA
agtacagtgccggg

SEQ ID NO: 722 - DNA, 14 bp, synthetic construct
  source 1..14: mol_type = other DNA
ccgtaataatctgt

SEQ ID NO: 723 - DNA, 14 bp, synthetic construct
  source 1..14: mol_type = other DNA
cccattcagttcac

SEQ ID NO: 724 - DNA, 14 bp, synthetic construct
  source 1..14: mol_type = other DNA
cggtacccattcag

SEQ ID NO: 725 - DNA, 14 bp, synthetic construct
  source 1..14: mol_type = other DNA
atagatgaacagtc

SEQ ID NO: 726 - DNA, 14 bp, synthetic construct
  source 1..14: mol_type = other DNA
acaatctagcatag

SEQ ID NO: 727 - DNA, 14 bp, synthetic construct
  source 1..14: mol_type = other DNA
tggactaccagtaa

SEQ ID NO: 728 - DNA, 14 bp, synthetic construct
  source 1..14: mol_type = other DNA
tacgagccatgaca

SEQ ID NO: 729 - DNA, 14 bp, synthetic construct
  source 1..14: mol_type = other DNA
gatagctacgagcc

SEQ ID NO: 730 - DNA, 14 bp, synthetic construct
  source 1..14: mol_type = other DNA
gggatagctacgag

SEQ ID NO: 731 - DNA, 14 bp, synthetic construct
  source 1..14: mol_type = other DNA
gagggatagctacg

SEQ ID NO: 732 - DNA, 14 bp, synthetic construct
  source 1..14: mol_type = other DNA
catgagcggtgtca

SEQ ID NO: 733 - DNA, 14 bp, synthetic construct
  source 1..14: mol_type = other DNA
tcaactatgtgtaa

SEQ ID NO: 734 - DNA, 14 bp, synthetic construct
  source 1..14: mol_type = other DNA
acgtgcttttatgt

SEQ ID NO: 735 - DNA, 14 bp, synthetic construct
  source 1..14: mol_type = other DNA
cacaaaacgtgctt

SEQ ID NO: 736 - DNA, 14 bp, synthetic construct
  source 1..14: mol_type = other DNA
gtaccacaaaacgt

SEQ ID NO: 737 - DNA, 14 bp, synthetic construct
  source 1..14: mol_type = other DNA
gcgtaccacaaaac

SEQ ID NO: 738 - DNA, 14 bp, synthetic construct
  source 1..14: mol_type = other DNA
caaggcgtaccaca

SEQ ID NO: 739 - DNA, 14 bp, synthetic construct
  source 1..14: mol_type = other DNA
aaaccaaggcgtac

SEQ ID NO: 740 - DNA, 14 bp, synthetic construct
  source 1..14: mol_type = other DNA
atacccatcctatt

SEQ ID NO: 741 - DNA, 14 bp, synthetic construct
  source 1..14: mol_type = other DNA
gttgcttaggaact

SEQ ID NO: 742 - DNA, 14 bp, synthetic construct
  source 1..14: mol_type = other DNA
ggtagatgttctat

SEQ ID NO: 743 - DNA, 14 bp, synthetic construct
  source 1..14: mol_type = other DNA
catacgctttatga

SEQ ID NO: 744 - DNA, 14 bp, synthetic construct
  source 1..14: mol_type = other DNA
tacatacgctttat

SEQ ID NO: 745 - DNA, 14 bp, synthetic construct
  source 1..14: mol_type = other DNA
acagtacatacgct

SEQ ID NO: 746 - DNA, 14 bp, synthetic construct
  source 1..14: mol_type = other DNA
aacagtacatacgc

SEQ ID NO: 747 - DNA, 14 bp, synthetic construct
  source 1..14: mol_type = other DNA
atcgtgatgtacat

SEQ ID NO: 748 - DNA, 14 bp, synthetic construct
  source 1..14: mol_type = other DNA
actgtactatcgtg

SEQ ID NO: 749 - DNA, 14 bp, synthetic construct
  source 1..14: mol_type = other DNA
ataggatagactgc

SEQ ID NO: 750 - DNA, 14 bp, synthetic construct
  source 1..14: mol_type = other DNA
ggacgcttttcaaa

SEQ ID NO: 751 - DNA, 14 bp, synthetic construct
  source 1..14: mol_type = other DNA
atgttttaggacgc

SEQ ID NO: 752 - DNA, 14 bp, synthetic construct
  source 1..14: mol_type = other DNA
ctagtaagttggct

SEQ ID NO: 753 - DNA, 14 bp, synthetic construct
  source 1..14: mol_type = other DNA
gcggctctagtaag

SEQ ID NO: 754 - DNA, 14 bp, synthetic construct
  source 1..14: mol_type = other DNA
tgtagcggctctag

SEQ ID NO: 755 - DNA, 14 bp, synthetic construct
  source 1..14: mol_type = other DNA
tagttgtagcggct

SEQ ID NO: 756 - DNA, 14 bp, synthetic construct
  source 1..14: mol_type = other DNA
attagttgtagcgg

SEQ ID NO: 757 - DNA, 14 bp, synthetic construct
  source 1..14: mol_type = other DNA
tctatcatgattag

SEQ ID NO: 758 - DNA, 14 bp, synthetic construct
  source 1..14: mol_type = other DNA
agccgaagacatac

SEQ ID NO: 759 - DNA, 14 bp, synthetic construct
  source 1..14: mol_type = other DNA
ttagccgaagacat

SEQ ID NO: 760 - DNA, 14 bp, synthetic construct
  source 1..14: mol_type = other DNA
cattagccgaagac

SEQ ID NO: 761 - DNA, 14 bp, synthetic construct
  source 1..14: mol_type = other DNA
aacacattagccga

SEQ ID NO: 762 - DNA, 14 bp, synthetic construct
  source 1..14: mol_type = other DNA
tattgaaggctaac

SEQ ID NO: 763 - DNA, 14 bp, synthetic construct
  source 1..14: mol_type = other DNA
gactagataggttg

SEQ ID NO: 764 - DNA, 14 bp, synthetic construct
  source 1..14: mol_type = other DNA
agactagataggtt

SEQ ID NO: 765 - DNA, 14 bp, synthetic construct
  source 1..14: mol_type = other DNA
atagactagatagg

SEQ ID NO: 766 - DNA, 14 bp, synthetic construct
  source 1..14: mol_type = other DNA
ttatagactagata

SEQ ID NO: 767 - DNA, 14 bp, synthetic construct
  source 1..14: mol_type = other DNA
tggtctcctagaac

SEQ ID NO: 768 - DNA, 14 bp, synthetic construct
  source 1..14: mol_type = other DNA
taagggcatccaat

SEQ ID NO: 769 - DNA, 14 bp, synthetic construct
  source 1..14: mol_type = other DNA
tgtcaactagttaa

SEQ ID NO: 770 - DNA, 14 bp, synthetic construct
  source 1..14: mol_type = other DNA
tgtaatgtccagga

SEQ ID NO: 771 - DNA, 14 bp, synthetic construct
  source 1..14: mol_type = other DNA
acccccttgtaatg

SEQ ID NO: 772 - DNA, 14 bp, synthetic construct
  source 1..14: mol_type = other DNA
catttacccccttg

SEQ ID NO: 773 - DNA, 14 bp, synthetic construct
  source 1..14: mol_type = other DNA
gcgaactctcaatg

SEQ ID NO: 774 - DNA, 14 bp, synthetic construct
  source 1..14: mol_type = other DNA
attaagcgaactct

SEQ ID NO: 775 - DNA, 14 bp, synthetic construct
  source 1..14: mol_type = other DNA
agtaacctactaaa

SEQ ID NO: 776 - DNA, 14 bp, synthetic construct
  source 1..14: mol_type = other DNA
agaatagtaaccta

SEQ ID NO: 777 - DNA, 14 bp, synthetic construct
  source 1..14: mol_type = other DNA
ttagtgtggcatgc

SEQ ID NO: 778 - DNA, 14 bp, synthetic construct
  source 1..14: mol_type = other DNA
cagctgacatagta

SEQ ID NO: 779 - DNA, 14 bp, synthetic construct
  source 1..14: mol_type = other DNA
tcataatatagcct

SEQ ID NO: 780 - DNA, 14 bp, synthetic construct
  source 1..14: mol_type = other DNA
ggtttacataaggt

SEQ ID NO: 781 - DNA, 14 bp, synthetic construct
  source 1..14: mol_type = other DNA
cgtagtcacacaat

SEQ ID NO: 782 - DNA, 14 bp, synthetic construct
  source 1..14: mol_type = other DNA
ttgcgtagtcacac

SEQ ID NO: 783 - DNA, 14 bp, synthetic construct
  source 1..14: mol_type = other DNA
cctatacttgcgta

SEQ ID NO: 784 - DNA, 14 bp, synthetic construct
  source 1..14: mol_type = other DNA
atgtataagggatt

SEQ ID NO: 785 - DNA, 14 bp, synthetic construct
  source 1..14: mol_type = other DNA
ttaagtagagctca

SEQ ID NO: 786 - DNA, 14 bp, synthetic construct
  source 1..14: mol_type = other DNA
agtcgacagaaaat

SEQ ID NO: 787 - DNA, 14 bp, synthetic construct
  source 1..14: mol_type = other DNA
gctcacagtcgaca

SEQ ID NO: 788 - DNA, 14 bp, synthetic construct
  source 1..14: mol_type = other DNA
tgctcacagtcgac

SEQ ID NO: 789 - DNA, 14 bp, synthetic construct
  source 1..14: mol_type = other DNA
acttaatgcactat

SEQ ID NO: 790 - DNA, 14 bp, synthetic construct
  source 1..14: mol_type = other DNA
tacttaatgcacta

SEQ ID NO: 791 - DNA, 14 bp, synthetic construct
  source 1..14: mol_type = other DNA
gacccactgttagc

SEQ ID NO: 792 - DNA, 14 bp, synthetic construct
  source 1..14: mol_type = other DNA
tgacccactgttag

SEQ ID NO: 793 - DNA, 14 bp, synthetic construct
  source 1..14: mol_type = other DNA
tgttatgcatcttg

SEQ ID NO: 794 - DNA, 14 bp, synthetic construct
  source 1..14: mol_type = other DNA
cctaagccacacca

SEQ ID NO: 795 - DNA, 14 bp, synthetic construct
  source 1..14: mol_type = other DNA
cacgtctcaactaa

SEQ ID NO: 796 - DNA, 14 bp, synthetic construct
  source 1..14: mol_type = other DNA
agcaaataaccgtg

SEQ ID NO: 797 - DNA, 14 bp, synthetic construct
  source 1..14: mol_type = other DNA
tagcaaataaccgt

SEQ ID NO: 798 - DNA, 14 bp, synthetic construct
  source 1..14: mol_type = other DNA
ccgaccgacagagc

SEQ ID NO: 799 - DNA, 14 bp, synthetic construct
  source 1..14: mol_type = other DNA
atcaaagaccgacc

SEQ ID NO: 800 - DNA, 14 bp, synthetic construct
  source 1..14: mol_type = other DNA
acatcaaagaccga

SEQ ID NO: 801 - DNA, 14 bp, synthetic construct
  source 1..14: mol_type = other DNA
gttaggtacaaagg

SEQ ID NO: 802 - DNA, 14 bp, synthetic construct
  source 1..14: mol_type = other DNA
acatggttaggtac

SEQ ID NO: 803 - DNA, 14 bp, synthetic construct
  source 1..14: mol_type = other DNA
agccgttattttca

SEQ ID NO: 804 - DNA, 14 bp, synthetic construct
  source 1..14: mol_type = other DNA
cagccgttattttc

SEQ ID NO: 805 - DNA, 14 bp, synthetic construct
  source 1..14: mol_type = other DNA
aaaataacagccgt

SEQ ID NO: 806 - DNA, 14 bp, synthetic construct
  source 1..14: mol_type = other DNA
agtttatcttagta

SEQ ID NO: 807 - DNA, 14 bp, synthetic construct
  source 1..14: mol_type = other DNA
ggctatgtaaggca

SEQ ID NO: 808 - DNA, 14 bp, synthetic construct
  source 1..14: mol_type = other DNA
aatttcataatgcc

SEQ ID NO: 809 - DNA, 14 bp, synthetic construct
  source 1..14: mol_type = other DNA
ttacaacactgtcc

SEQ ID NO: 810 - DNA, 14 bp, synthetic construct
  source 1..14: mol_type = other DNA
attcattacaacac

SEQ ID NO: 811 - DNA, 14 bp, synthetic construct
  source 1..14: mol_type = other DNA
aaattgggatctgc

SEQ ID NO: 812 - DNA, 14 bp, synthetic construct
  source 1..14: mol_type = other DNA
atatgccactcaac

SEQ ID NO: 813 - DNA, 14 bp, synthetic construct
  source 1..14: mol_type = other DNA
gcaatttctattgg

SEQ ID NO: 814 - DNA, 14 bp, synthetic construct
  source 1..14: mol_type = other DNA
gagcaatttctatt

SEQ ID NO: 815 - DNA, 14 bp, synthetic construct
  source 1..14: mol_type = other DNA
gctggttctatttc

SEQ ID NO: 816 - DNA, 14 bp, synthetic construct
  source 1..14: mol_type = other DNA
gtttcgtggttcta

SEQ ID NO: 817 - DNA, 14 bp, synthetic construct
  source 1..14: mol_type = other DNA
tgcacttgttcctg

SEQ ID NO: 818 - DNA, 14 bp, synthetic construct
  source 1..14: mol_type = other DNA
gagtgggatgcact

SEQ ID NO: 819 - DNA, 14 bp, synthetic construct
  source 1..14: mol_type = other DNA
tagcagattgtgca

SEQ ID NO: 820 - DNA, 14 bp, synthetic construct
  source 1..14: mol_type = other DNA
tcgtttttctatag

SEQ ID NO: 821 - DNA, 14 bp, synthetic construct
  source 1..14: mol_type = other DNA
cgttttcgtttttc

SEQ ID NO: 822 - DNA, 14 bp, synthetic construct
  source 1..14: mol_type = other DNA
cttcgttttcgttt

SEQ ID NO: 823 - DNA, 14 bp, synthetic construct
  source 1..14: mol_type = other DNA
ttcttcgttttcgt

SEQ ID NO: 824 - DNA, 14 bp, synthetic construct
  source 1..14: mol_type = other DNA
atggtagtccactt

SEQ ID NO: 825 - DNA, 14 bp, synthetic construct
  source 1..14: mol_type = other DNA
tacatggtagtcca

SEQ ID NO: 826 - DNA, 14 bp, synthetic construct
  source 1..14: mol_type = other DNA
gatacatggtagtc

SEQ ID NO: 827 - DNA, 14 bp, synthetic construct
  source 1..14: mol_type = other DNA
gacactttaatgtc

SEQ ID NO: 828 - DNA, 14 bp, synthetic construct
  source 1..14: mol_type = other DNA
gagagaaaccaaac

SEQ ID NO: 829 - DNA, 14 bp, synthetic construct
  source 1..14: mol_type = other DNA
atagtagtttaatt

SEQ ID NO: 830 - DNA, 14 bp, synthetic construct
  source 1..14: mol_type = other DNA
gcgatttcagttct

SEQ ID NO: 831 - DNA, 14 bp, synthetic construct
  source 1..14: mol_type = other DNA
atatttaattcccg

SEQ ID NO: 832 - DNA, 14 bp, synthetic construct
  source 1..14: mol_type = other DNA
acacttttatgatc

SEQ ID NO: 833 - DNA, 14 bp, synthetic construct
  source 1..14: mol_type = other DNA
tcgaaacactttta

SEQ ID NO: 834 - DNA, 14 bp, synthetic construct
  source 1..14: mol_type = other DNA
ttctcgaaacactt

SEQ ID NO: 835 - DNA, 14 bp, synthetic construct
  source 1..14: mol_type = other DNA
ttcatgtatatctg

SEQ ID NO: 836 - DNA, 14 bp, synthetic construct
  source 1..14: mol_type = other DNA
gtcaattcatgtat

SEQ ID NO: 837 - DNA, 14 bp, synthetic construct
  source 1..14: mol_type = other DNA
cttcaatcaaacct

SEQ ID NO: 838 - DNA, 14 bp, synthetic construct
  source 1..14: mol_type = other DNA
gtgataatgtttca

SEQ ID NO: 839 - DNA, 14 bp, synthetic construct
  source 1..14: mol_type = other DNA
gtgtagagcaactg

SEQ ID NO: 840 - DNA, 14 bp, synthetic construct
  source 1..14: mol_type = other DNA
aagtgtagagcaac

SEQ ID NO: 841 - DNA, 14 bp, synthetic construct
  source 1..14: mol_type = other DNA
ctgaaagtgtagag

SEQ ID NO: 842 - DNA, 14 bp, synthetic construct
  source 1..14: mol_type = other DNA
agcataagacgtgg

SEQ ID NO: 843 - DNA, 14 bp, synthetic construct
  source 1..14: mol_type = other DNA
acagtgatacactg

SEQ ID NO: 844 - DNA, 14 bp, synthetic construct
  source 1..14: mol_type = other DNA
gtaactcaaagtag

SEQ ID NO: 845 - DNA, 14 bp, synthetic construct
  source 1..14: mol_type = other DNA
ttgttcttcactac

SEQ ID NO: 846 - DNA, 14 bp, synthetic construct
  source 1..14: mol_type = other DNA
aatagcttggttca

SEQ ID NO: 847 - DNA, 14 bp, synthetic construct
  source 1..14: mol_type = other DNA
agtaatagcttggt

SEQ ID NO: 848 - DNA, 14 bp, synthetic construct
  source 1..14: mol_type = other DNA
cttgtaaattcggt

SEQ ID NO: 849 - DNA, 14 bp, synthetic construct
  source 1..14: mol_type = other DNA
cgacgtctaggtga

SEQ ID NO: 850 - DNA, 14 bp, synthetic construct
  source 1..14: mol_type = other DNA
taattggtgactat

SEQ ID NO: 851 - DNA, 14 bp, synthetic construct
  source 1..14: mol_type = other DNA
cttaattggtgact

SEQ ID NO: 852 - DNA, 14 bp, synthetic construct
  source 1..14: mol_type = other DNA
cccatccatcgata

SEQ ID NO: 853 - DNA, 14 bp, synthetic construct
  source 1..14: mol_type = other DNA
aattcattacaaca

SEQ ID NO: 854 - DNA, 14 bp, synthetic construct
  source 1..14: mol_type = other DNA
cgattcttgtcaat

SEQ ID NO: 855 - DNA, 14 bp, synthetic construct
  source 1..14: mol_type = other DNA
caccgattcttgtc

SEQ ID NO: 856 - DNA, 14 bp, synthetic construct
  source 1..14: mol_type = other DNA
ttggcaccgattct

SEQ ID NO: 857 - DNA, 14 bp, synthetic construct
  source 1..14: mol_type = other DNA
atgtttggcaccga

SEQ ID NO: 858 - DNA, 14 bp, synthetic construct
  source 1..14: mol_type = other DNA
gccgttcatgtttg

SEQ ID NO: 859 - DNA, 14 bp, synthetic construct
  source 1..14: mol_type = other DNA
tgattaggtttaca

SEQ ID NO: 860 - DNA, 14 bp, synthetic construct
  source 1..14: mol_type = other DNA
cactactgtaacct

SEQ ID NO: 861 - DNA, 14 bp, synthetic construct
  source 1..14: mol_type = other DNA
catgacaaattttt

SEQ ID NO: 862 - DNA, 14 bp, synthetic construct
  source 1..14: mol_type = other DNA
gctcacaagtatct

SEQ ID NO: 863 - DNA, 14 bp, synthetic construct
  source 1..14: mol_type = other DNA
atgcatgcttgtgc

SEQ ID NO: 864 - DNA, 14 bp, synthetic construct
  source 1..14: mol_type = other DNA
tcaaatgcatgctt

SEQ ID NO: 865 - DNA, 14 bp, synthetic construct
  source 1..14: mol_type = other DNA
gcagcaaatattat

SEQ ID NO: 866 - DNA, 14 bp, synthetic construct
  source 1..14: mol_type = other DNA
ttcgatctagagta

SEQ ID NO: 867 - DNA, 14 bp, synthetic construct
  source 1..14: mol_type = other DNA
aacattcgatctag

SEQ ID NO: 868 - DNA, 14 bp, synthetic construct
  source 1..14: mol_type = other DNA
ggtaatctcagtgg

SEQ ID NO: 869 - DNA, 14 bp, synthetic construct
  source 1..14: mol_type = other DNA
caaaacgatatact

SEQ ID NO: 870 - DNA, 14 bp, synthetic construct
  source 1..14: mol_type = other DNA
ctacaaaacgatat

SEQ ID NO: 871 - DNA, 14 bp, synthetic construct
  source 1..14: mol_type = other DNA
acaatgttttcctc

SEQ ID NO: 872 - DNA, 14 bp, synthetic construct
  source 1..14: mol_type = other DNA
tgcaagttgtcttc

SEQ ID NO: 873 - DNA, 14 bp, synthetic construct
  source 1..14: mol_type = other DNA
cctttaataatggg

SEQ ID NO: 874 - DNA, 14 bp, synthetic construct
  source 1..14: mol_type = other DNA
tgttaacctttcaa

SEQ ID NO: 875 - DNA, 14 bp, synthetic construct
  source 1..14: mol_type = other DNA
attgggatctgcat

SEQ ID NO: 876 - DNA, 14 bp, synthetic construct
  source 1..14: mol_type = other DNA
acgatatgtggtaa

SEQ ID NO: 877 - DNA, 14 bp, synthetic construct
  source 1..14: mol_type = other DNA
gctcagcaattcct

SEQ ID NO: 878 - DNA, 14 bp, synthetic construct
  source 1..14: mol_type = other DNA
gaatttcaaaccgt

SEQ ID NO: 879 - DNA, 14 bp, synthetic construct
  source 1..14: mol_type = other DNA
cgtcagtaggttct

SEQ ID NO: 880 - DNA, 14 bp, synthetic construct
  source 1..14: mol_type = other DNA
aggtctgcactgat

SEQ ID NO: 881 - DNA, 14 bp, synthetic construct
  source 1..14: mol_type = other DNA
tcttttaaggtctg

SEQ ID NO: 882 - DNA, 14 bp, synthetic construct
  source 1..14: mol_type = other DNA
aatcttttaaggtc

SEQ ID NO: 883 - DNA, 14 bp, synthetic construct
  source 1..14: mol_type = other DNA
gcgaaatcttttaa

SEQ ID NO: 884 - DNA, 14 bp, synthetic construct
  source 1..14: mol_type = other DNA
cctaagttgtactg

SEQ ID NO: 885 - DNA, 14 bp, synthetic construct
  source 1..14: mol_type = other DNA
gatcctaagttgta

SEQ ID NO: 886 - DNA, 14 bp, synthetic construct
  source 1..14: mol_type = other DNA
ccctcttacacttg

SEQ ID NO: 887 - DNA, 14 bp, synthetic construct
  source 1..14: mol_type = other DNA
gactctacccattt

SEQ ID NO: 888 - DNA, 14 bp, synthetic construct
  source 1..14: mol_type = other DNA
agcaattgactcta

SEQ ID NO: 889 - DNA, 14 bp, synthetic construct
  source 1..14: mol_type = other DNA
ttagcaattgactc

SEQ ID NO: 890 - DNA, 14 bp, synthetic construct
  source 1..14: mol_type = other DNA
atcttagcaattga

SEQ ID NO: 891 - DNA, 14 bp, synthetic construct
  source 1..14: mol_type = other DNA
atgatcttagcaat

SEQ ID NO: 892 - DNA, 14 bp, synthetic construct
  source 1..14: mol_type = other DNA
ctgatgatcttagc

SEQ ID NO: 893 - DNA, 14 bp, synthetic construct
  source 1..14: mol_type = other DNA
tggcttactccgtt

SEQ ID NO: 894 - DNA, 14 bp, synthetic construct
  source 1..14: mol_type = other DNA
taatattatggctt

SEQ ID NO: 895 - DNA, 14 bp, synthetic construct
  source 1..14: mol_type = other DNA
cattcaattggtgg

SEQ ID NO: 896 - DNA, 14 bp, synthetic construct
  source 1..14: mol_type = other DNA
atgaagtgtcatga

SEQ ID NO: 897 - DNA, 14 bp, synthetic construct
  source 1..14: mol_type = other DNA
ctgacgtgcaattt

SEQ ID NO: 898 - DNA, 14 bp, synthetic construct
  source 1..14: mol_type = other DNA
tcagctgacgtgca

SEQ ID NO: 899 - DNA, 14 bp, synthetic construct
  source 1..14: mol_type = other DNA
tgtcagctgacgtg

SEQ ID NO: 900 - DNA, 14 bp, synthetic construct
  source 1..14: mol_type = other DNA
gacggttgaacttt

SEQ ID NO: 901 - DNA, 14 bp, synthetic construct
  source 1..14: mol_type = other DNA
acaagttcagacgg

SEQ ID NO: 902 - DNA, 14 bp, synthetic construct
  source 1..14: mol_type = other DNA
atggcgaatcattt

SEQ ID NO: 903 - DNA, 14 bp, synthetic construct
  source 1..14: mol_type = other DNA
tggtatggcgaatc

SEQ ID NO: 904 - DNA, 14 bp, synthetic construct
  source 1..14: mol_type = other DNA
tgtggtatggcgaa

SEQ ID NO: 905 - DNA, 14 bp, synthetic construct
  source 1..14: mol_type = other DNA
agatttgtggtatg

SEQ ID NO: 906 - DNA, 14 bp, synthetic construct
  source 1..14: mol_type = other DNA
gccacccgttcttc

SEQ ID NO: 907 - DNA, 14 bp, synthetic construct
  source 1..14: mol_type = other DNA
gcagaatttctata

SEQ ID NO: 908 - DNA, 14 bp, synthetic construct
  source 1..14: mol_type = other DNA
gccattgaaattat

SEQ ID NO: 909 - DNA, 14 bp, synthetic construct
  source 1..14: mol_type = other DNA
gaatttactgcact

SEQ ID NO: 910 - DNA, 14 bp, synthetic construct
  source 1..14: mol_type = other DNA
gacactgaatttac

SEQ ID NO: 911 - DNA, 14 bp, synthetic construct
  source 1..14: mol_type = other DNA
tgacttaattccac

SEQ ID NO: 912 - DNA, 14 bp, synthetic construct
  source 1..14: mol_type = other DNA
gttttactaggtat

SEQ ID NO: 913 - DNA, 14 bp, synthetic construct
  source 1..14: mol_type = other DNA
aagttttactaggt

SEQ ID NO: 914 - DNA, 14 bp, synthetic construct
  source 1..14: mol_type = other DNA
ttcttcggtcttca

SEQ ID NO: 915 - DNA, 14 bp, synthetic construct
  source 1..14: mol_type = other DNA
tcccttcttcggtc

SEQ ID NO: 916 - DNA, 14 bp, synthetic construct
  source 1..14: mol_type = other DNA
atcattattccctt

SEQ ID NO: 917 - DNA, 14 bp, synthetic construct
  source 1..14: mol_type = other DNA
aatctttccctttc

SEQ ID NO: 918 - DNA, 14 bp, synthetic construct
  source 1..14: mol_type = other DNA
ccgtaaacagtaag

SEQ ID NO: 919 - DNA, 14 bp, synthetic construct
  source 1..14: mol_type = other DNA
tctatccgtaaaca

SEQ ID NO: 920 - DNA, 14 bp, synthetic construct
  source 1..14: mol_type = other DNA
ctggttctatccgt

SEQ ID NO: 921 - DNA, 14 bp, synthetic construct
  source 1..14: mol_type = other DNA
cctcatatctggtt

SEQ ID NO: 922 - DNA, 14 bp, synthetic construct
  source 1..14: mol_type = other DNA
gggttaaggttttc

SEQ ID NO: 923 - DNA, 14 bp, synthetic construct
  source 1..14: mol_type = other DNA
tcccatggggttaa

SEQ ID NO: 924 - DNA, 14 bp, synthetic construct
  source 1..14: mol_type = other DNA
tttcgaggttcaat

SEQ ID NO: 925 - DNA, 14 bp, synthetic construct
  source 1..14: mol_type = other DNA
ggtggctgtttgca

SEQ ID NO: 926 - DNA, 14 bp, synthetic construct
  source 1..14: mol_type = other DNA
tcgaggtggctgtt

SEQ ID NO: 927 - DNA, 14 bp, synthetic construct
  source 1..14: mol_type = other DNA
gttgatttcctagg

SEQ ID NO: 928 - DNA, 14 bp, synthetic construct
  source 1..14: mol_type = other DNA
tgttaggccttatt

SEQ ID NO: 929 - DNA, 14 bp, synthetic construct
  source 1..14: mol_type = other DNA
tgtcggcctgtgtt

SEQ ID NO: 930 - DNA, 14 bp, synthetic construct
  source 1..14: mol_type = other DNA
catgtcggcctgtg

SEQ ID NO: 931 - DNA, 14 bp, synthetic construct
  source 1..14: mol_type = other DNA
gtagagccatgtcg

SEQ ID NO: 932 - DNA, 14 bp, synthetic construct
  source 1..14: mol_type = other DNA
gttgggtgacctcg

SEQ ID NO: 933 - DNA, 14 bp, synthetic construct
  source 1..14: mol_type = other DNA
ctctttctaatggt

SEQ ID NO: 934 - DNA, 14 bp, synthetic construct
  source 1..14: mol_type = other DNA
ccgactaaagctta

SEQ ID NO: 935 - DNA, 14 bp, synthetic construct
  source 1..14: mol_type = other DNA
caatccgactaaag

SEQ ID NO: 936 - DNA, 14 bp, synthetic construct
  source 1..14: mol_type = other DNA
agcaatccgactaa

SEQ ID NO: 937 - DNA, 14 bp, synthetic construct
  source 1..14: mol_type = other DNA
ttcagcaatccgac

SEQ ID NO: 938 - DNA, 14 bp, synthetic construct
  source 1..14: mol_type = other DNA
tgttgattccagct

SEQ ID NO: 939 - DNA, 14 bp, synthetic construct
  source 1..14: mol_type = other DNA
ctgacactgttgat

SEQ ID NO: 940 - DNA, 14 bp, synthetic construct
  source 1..14: mol_type = other DNA
tacactaaggtctg

SEQ ID NO: 941 - DNA, 14 bp, synthetic construct
  source 1..14: mol_type = other DNA
ggagcaaagatgct

SEQ ID NO: 942 - DNA, 14 bp, synthetic construct
  source 1..14: mol_type = other DNA
gggctcttcactta

SEQ ID NO: 943 - DNA, 14 bp, synthetic construct
  source 1..14: mol_type = other DNA
aaacttttttcgag

SEQ ID NO: 944 - DNA, 14 bp, synthetic construct
  source 1..14: mol_type = other DNA
ggtggaatagcagg

SEQ ID NO: 945 - DNA, 14 bp, synthetic construct
  source 1..14: mol_type = other DNA
gaggctgtctcggt

SEQ ID NO: 946 - DNA, 14 bp, synthetic construct
  source 1..14: mol_type = other DNA
ggtggactatgcag

SEQ ID NO: 947 - DNA, 14 bp, synthetic construct
  source 1..14: mol_type = other DNA
gtggctgaagatta

SEQ ID NO: 948 - DNA, 14 bp, synthetic construct
  source 1..14: mol_type = other DNA
cacgtactaatgtc

SEQ ID NO: 949 - DNA, 14 bp, synthetic construct
  source 1..14: mol_type = other DNA
gcttggcgaatttg

SEQ ID NO: 950 - DNA, 14 bp, synthetic construct
  source 1..14: mol_type = other DNA
cgcggtacccttgg

SEQ ID NO: 951 - DNA, 14 bp, synthetic construct
  source 1..14: mol_type = other DNA
tcgacgcggtaccc

SEQ ID NO: 952 - DNA, 14 bp, synthetic construct
  source 1..14: mol_type = other DNA
catcgacgcggtac

SEQ ID NO: 953 - DNA, 14 bp, synthetic construct
  source 1..14: mol_type = other DNA
agcatcgacgcggt

SEQ ID NO: 954 - DNA, 14 bp, synthetic construct
  source 1..14: mol_type = other DNA
ctagaactgagcac

SEQ ID NO: 955 - DNA, 14 bp, synthetic construct
  source 1..14: mol_type = other DNA
gattattctgacta

SEQ ID NO: 956 - DNA, 14 bp, synthetic construct
  source 1..14: mol_type = other DNA
ggatgagcaagatt

SEQ ID NO: 957 - DNA, 14 bp, synthetic construct
  source 1..14: mol_type = other DNA
agatgagggcttga

SEQ ID NO: 958 - DNA, 14 bp, synthetic construct
  source 1..14: mol_type = other DNA
ttggcagatgaggg

SEQ ID NO: 959 - DNA, 14 bp, synthetic construct
  source 1..14: mol_type = other DNA
ggcagttttggcag

SEQ ID NO: 960 - DNA, 14 bp, synthetic construct
  source 1..14: mol_type = other DNA
ttgtaagtctttgg

SEQ ID NO: 961 - DNA, 14 bp, synthetic construct
  source 1..14: mol_type = other DNA
ccgtttgtaagtct

SEQ ID NO: 962 - DNA, 14 bp, synthetic construct
  source 1..14: mol_type = other DNA
gctcccgtttgtaa

SEQ ID NO: 963 - DNA, 14 bp, synthetic construct
  source 1..14: mol_type = other DNA
gtacaatggggggt

SEQ ID NO: 964 - DNA, 14 bp, synthetic construct
  source 1..14: mol_type = other DNA
aaaggcagtctgta

SEQ ID NO: 965 - DNA, 14 bp, synthetic construct
  source 1..14: mol_type = other DNA
tctagcaaaggcag

SEQ ID NO: 966 - DNA, 14 bp, synthetic construct
  source 1..14: mol_type = other DNA
gcattttctagcaa

SEQ ID NO: 967 - DNA, 14 bp, synthetic construct
  source 1..14: mol_type = other DNA
tggctaaggtcatt

SEQ ID NO: 968 - DNA, 14 bp, synthetic construct
  source 1..14: mol_type = other DNA
ataccattccagtg

SEQ ID NO: 969 - DNA, 14 bp, synthetic construct
  source 1..14: mol_type = other DNA
tgtaagagcattat

SEQ ID NO: 970 - DNA, 14 bp, synthetic construct
  source 1..14: mol_type = other DNA
aagcttatttgatc

SEQ ID NO: 971 - DNA, 14 bp, synthetic construct
  source 1..14: mol_type = other DNA
gataatggtgaagg

SEQ ID NO: 972 - DNA, 14 bp, synthetic construct
  source 1..14: mol_type = other DNA
atactggccttttg

SEQ ID NO: 973 - DNA, 14 bp, synthetic construct
  source 1..14: mol_type = other DNA
atcatgtcacttta

SEQ ID NO: 974 - DNA, 14 bp, synthetic construct
  source 1..14: mol_type = other DNA
tgcaatcatgtcac

SEQ ID NO: 975 - DNA, 14 bp, synthetic construct
  source 1..14: mol_type = other DNA
catctattactcaa

SEQ ID NO: 976 - DNA, 14 bp, synthetic construct
  source 1..14: mol_type = other DNA
tggtcaaagaggtt

SEQ ID NO: 977 - DNA, 14 bp, synthetic construct
  source 1..14: mol_type = other DNA
gttggtgttattca

SEQ ID NO: 978 - DNA, 14 bp, synthetic construct
  source 1..14: mol_type = other DNA
cagttggtgttatt

SEQ ID NO: 979 - DNA, 14 bp, synthetic construct
  source 1..14: mol_type = other DNA
aagaacagttggtg

SEQ ID NO: 980 - DNA, 14 bp, synthetic construct
  source 1..14: mol_type = other DNA
atcattgggttatg

SEQ ID NO: 981 - DNA, 14 bp, synthetic construct
  source 1..14: mol_type = other DNA
aaaggcacatctga

SEQ ID NO: 982 - DNA, 14 bp, synthetic construct
  source 1..14: mol_type = other DNA
atataggttattag

SEQ ID NO: 983 - DNA, 14 bp, synthetic construct
  source 1..14: mol_type = other DNA
tataggcctcaagt

SEQ ID NO: 984 - DNA, 14 bp, synthetic construct
  source 1..14: mol_type = other DNA
gattgtattatagg

SEQ ID NO: 985 - DNA, 14 bp, synthetic construct
  source 1..14: mol_type = other DNA
gcagattgtattat

SEQ ID NO: 986 - DNA, 14 bp, synthetic construct
  source 1..14: mol_type = other DNA
agtagcagattgta

SEQ ID NO: 987 - DNA, 14 bp, synthetic construct
  source 1..14: mol_type = other DNA
gtaaagtagcagat

SEQ ID NO: 988 - DNA, 14 bp, synthetic construct
  source 1..14: mol_type = other DNA
tttatgaattagta

SEQ ID NO: 989 - DNA, 14 bp, synthetic construct
  source 1..14: mol_type = other DNA
cagttggttcctat

SEQ ID NO: 990 - DNA, 14 bp, synthetic construct
  source 1..14: mol_type = other DNA
caaggcagttggtt

SEQ ID NO: 991 - DNA, 14 bp, synthetic construct
  source 1..14: mol_type = other DNA
tgaaggcaaggcag

SEQ ID NO: 992 - DNA, 14 bp, synthetic construct
  source 1..14: mol_type = other DNA
actaggtcttgaag

SEQ ID NO: 993 - DNA, 14 bp, synthetic construct
  source 1..14: mol_type = other DNA
aagttactaggtct

SEQ ID NO: 994 - DNA, 14 bp, synthetic construct
  source 1..14: mol_type = other DNA
acgagatttataga

SEQ ID NO: 995 - DNA, 14 bp, synthetic construct
  source 1..14: mol_type = other DNA
aacacgagatttat

SEQ ID NO: 996 - DNA, 14 bp, synthetic construct
  source 1..14: mol_type = other DNA
gttaacacgagatt

SEQ ID NO: 997 - DNA, 14 bp, synthetic construct
  source 1..14: mol_type = other DNA
acttatctgattaa

SEQ ID NO: 998 - DNA, 14 bp, synthetic construct
  source 1..14: mol_type = other DNA
gtaaataaacaacg

SEQ ID NO: 999 - DNA, 14 bp, synthetic construct
  source 1..14: mol_type = other DNA
ttattaatcttgtt

SEQ ID NO: 1000 - DNA, 14 bp, synthetic construct
  source 1..14: mol_type = other DNA
actggggtgattta

SEQ ID NO: 1001 - DNA, 14 bp, synthetic construct
  source 1..14: mol_type = other DNA
attcatgtatatct

SEQ ID NO: 1002 - DNA, 14 bp, synthetic construct
  source 1..14: mol_type = other DNA
gttcatgtatatct

SEQ ID NO: 1003 - DNA, 14 bp, Synthetic construct
  source 1..14: mol_type = other DNA
aattcatgtatatc

SEQ ID NO: 1004 - DNA, 14 bp, Synthetic construct
  source 1..14: mol_type = other DNA
agttcatgtatatc